(19) United States
(12) Patent Application Publication
(10) Pub. No.: US 2023/0201262 A1
(43) Pub. Date: Jun. 29, 2023

(54) MYELOMA-SPECIFIC IMMUNITY REQUIRES THE DIFFERENTIATION OF STEM-LIKE MEMORY T CELLS IN THE BONE MARROW

(71) Applicant: Fred Hutchinson Cancer Center, Seattle, WA (US)

(72) Inventors: Simone Minnie, Seattle, WA (US); Geoffrey Hill, Seattle, WA (US)

(73) Assignee: Fred Hutchinson Cancer Center, Seattle, WA (US)

(21) Appl. No.: 18/062,522

(22) Filed: Dec. 6, 2022

Related U.S. Application Data
(60) Provisional application No. 63/286,392, filed on Dec. 6, 2021.

(51) Int. Cl. A61K 35/17; A61P 35/02; A61P 37/06; A61K 35/28

(52) U.S. Cl. CPC A61K 35/17; A61K 35/28; A61P 35/02; A61P 37/06; A61K 2035/122

(57) ABSTRACT

Methods for treating malignancies such as hematological malignancies, including myeloma, that are resistant to tissue transplant treatments and that can be characterized by an increased risk of relapse or graft-versus-host disease (GVHD). A method for treatment includes transplanting a tissue that includes T cells to a subject, enriching for a stem-like memory T cell phenotype in the T cells, and stimulating the T cells to enhance a graft-versus-tumor (GVT) response of the T cells. The enriching for the stem-like memory T cell phenotype can include depletion of exhausted alloreactive T cells with a post-transplant cyclophosphamide (PT-Cy) treatment and the stimulating the T cells can include an agonist immunotherapy, such as a decoy-resistant IL-18 (DR-18) treatment, to enhance the GVT response.

### CROSS-REFERENCE TO RELATED APPLICATION

This application claims the benefit of U.S. Provisional Application No. 63/286,392, filed Dec. 06, 2021, the disclosure of which is hereby incorporated by reference in its entirety.

### BACKGROUND

T cell exhaustion is a driver of loss of immunosurveillance in many cancers, including hematological malignancies. The use of immunotherapies, such as immune checkpoint inhibition (ICI), has been a successful strategy to enhance antitumor effects in certain solid tumor settings as well as some hematological malignancies. The importance of precursor exhausted and stem-like memory T cell subsets in generating a sustainable response to immunotherapies is becoming increasingly recognized, highlighting the need for methods for targeting these cell populations directly.

In hematological malignancies, particularly leukemias, allogeneic bone marrow transplantation (alloBMT) remains the only curative immunotherapy option. The curative potential of BMT is largely mediated by donor T cells recognizing recipient alloantigen comprising hematopoietic or tumor-specific antigens on the underlying malignancy, which is referred to as the graft-versus tumor (GVT) effect. However, alloBMT is limited by donor T cell recognition of alloantigen on normal tissue, a process known as graft-versus-host disease (GVHD), as well as relapse of the original malignancy attributable to immune escape.

Results from previous studies in which patients received PD-1 blockade after alloBMT have suggested this approach is associated with exacerbation of GVHD, consistent with the role of PD-1 in suppressing alloreactive donor T cell function. Although there are a number of described mechanisms for immune escape after alloBMT, some hematological malignancies (e.g., myeloma) are inherently resistant to GVT responses, and the cause of this has previously remained unknown, highlighting the need for developing a better understanding of these mechanisms.

Accordingly, there is a need for methods for eliciting a strong GVT effect without inducing lethal GVHD, which likely involves modulation of the T cell repertoire such that highly alloreactive T cells are eliminated before initiating immunotherapy and/or immunotherapy selectively targeting tumor-specific T cells. The present disclosure meets these and other long-felt and unmet needs in the art.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In a general aspect, the disclosure provides methods for treating a malignancy, such as a hematological malignancy, by modulating a T cell repertoire in a subject who has received a tissue transplant for treatment of the malignancy. The T cells can be treated with an agonist immunotherapy to enhance a graft-versus-tumor (GVT) response by the T cells and can be treated with a treatment to enrich for a stem-like memory T cell phenotype. In at least some embodiments, the treatment to enrich for the stem-like memory T cell phenotype can occur before the agonist immunotherapy. The methods can include transplanting a tissue that comprises a plurality of T cells to the subject, enriching for a stem-like memory T cell phenotype in the plurality of T cells, and stimulating the plurality of T cells to enhance a GVT response to treat the malignancy. Various malignancies, including myeloma and leukemia, are treatable by methods of the disclosure.

To enrich for the stem-like memory phenotype, at least a portion of the plurality of T cells can be depleted, e.g., with a treatment that can preferentially deplete cells that do not have the stem-like memory phenotype. As a non-limiting example, the enriching can include administering a post-transplant cyclophosphamide (PT-Cy) treatment to the subject.

A stem-like memory T cell phenotype of the T cells can be characterized by an increased chromatin accessibility in a cytokine signaling gene and/or an increased expression of interleukin-18 receptor (IL-18R), Transcription Factor 7 (TCF7), Transcription Factor 7 Like 2 (TCFL2), Krüppel-like transcription factor 2 (KLF2), Krüppel-like transcription factor 4 (KLF4), and/or Krüppel-like transcription factor 5 (KLF5) by at least a portion of the plurality of T cells.

To stimulate the T cells, the method can include administering an agonist immunotherapy (e.g., an anti-CD137 antibody treatment, a decoy-resistant IL-18 (DR-18) treatment, or both) to the subject for expansion of activated CD8 T cells, expansion of natural killer (NK) cells, or both. The expanded immune cells can exhibit a lower GVHD response and a higher GVT response.

The method can be combined with other methods or treatments for treating the malignancy or other diseases, conditions, or disorders of the subject. Such other treatments can include, for example, administering a donor lymphocyte infusion (DLI) to the subject for treating the malignancy.

In another aspect, the disclosure provides a method for enhancing a graft-versus-tumor (GVT) response of a plurality of T cells to treat a hematological malignancy of a subject that includes administering a decoy-resistant IL-18 (DR-18) treatment to the subject. The method can further include administering a treatment, such as a post-transplant cyclophosphamide (PT-Cy) treatment, to the subject to enrich for a stem-like memory T cell phenotype in the plurality of T cells.

### DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and many of the attendant advantages of this disclosure will become more readily appreciated as the same become better understood by reference to the following detailed description, when taken in conjunction with the accompanying drawings, wherein:

FIG. 1A shows an experimental design in which C57B⅙ (B6) recipients injected with MLL-AF9 (AML-bearing; D0) were lethally irradiated and transplanted with 5 × 10⁶ BM with 0.5 × 10⁶ CD4⁺ + 0.5 × 10⁶ CD8⁺ T cells from B6 (synBMT) or C3H.SW (alloBMT) donors to quantify the total number of circulating GFP⁺ AML cells to determine risk of death due to acute myeloid leukemia (AML) or graft-versus-host disease (GVHD).

FIG. 1B shows results from experiments in which AML-bearing recipients were bled weekly to quantify the total number of circulating GFP⁺ AML cells (left) and competing risk analysis was performed to determine risk of death due to acute myeloid leukemia (AML) or GVHD (right). n = 11/group from 2 experiments. Mann-Whitney U test for AML burden.

FIG. 1C shows an experimental design in which C57B⅙ (B6) recipients injected with Vk*MYC myeloma (MM-bearing; D-14) were lethally irradiated and transplanted with 5 × 10⁶ BM with 0.5 × 10⁶ CD4⁺ + 0.5 × 10⁶ CD8⁺ T cells from B6 (synBMT) or C3H.SW (alloBMT) donors to determine risk of death due to myeloma (MM) or graft-versus-host disease (GVHD); MM-bearing recipients were evaluated for tumor burden using M-band (G/A).

FIG. 1D shows results from experiments in which MM-bearing recipients were monitored for tumor burden using M-band (G/A). M-bands were modeled to calculate a predictive rate of tumor growth (solid line), with shaded confidence intervals and M-band relapse threshold shown as dotted line (left). Competing risk analysis was performed to determine risk of death due to myeloma (MM) or graft-versus-host disease (GVHD) (right). n = 20/group from 3 experiments. * p<0.05, ** p<0.01.

FIG. 2A shows results from experiments in which t-SNE analysis identified CD8 T cell clusters based on PD-1, TIGIT, TIM-3, DNAM-1, CD44 and CD62L expression at 2 weeks and 8 weeks posttransplant (n = 3-5).

FIG. 2B shows results from experiments showing CD4⁺ and CD8⁺ T cell number for alloBMT and synBMT groups.

FIG. 2C shows frequency of effector CD8 T cells (CD44⁺CD62L⁻, T_{EM}) and central memory T cells (CD44⁺CD62L⁺, T_{CM}) for alloBMT and synBMT groups.

FIG. 2D shows frequency of TIGIT⁺, PD-1⁺, TIM3⁺ CD8 T cells for alloBMT and synBMT groups.

FIG. 2E shows FACS plots of PD-L1 and CD155 expression on Vk12653 (red) and MLL-AF9 (blue) for alloBMT and synBMT groups.

FIG. 2F shows median overall survival analyzed with Log-rank test. Recipients were treated with 100 µg/mouse of anti-TIGIT (clone G2a; αTIGIT-G2a) or mIgG2a isotype control (cIg) twice a week from 2 weeks to 6 weeks posttransplant.

FIG. 2G shows M-band (log gamma/albumin) at 6 and 8 weeks after alloBMT. Recipients were treated with 100 µg/mouse of anti-TIGIT (clone G2a; αTIGIT-G2a) or mIgG2a isotype control (cIg) twice a week from 2 weeks to 6 weeks posttransplant.

FIG. 2H shows clinical score. Recipients were treated with 100 µg/mouse of anti-TIGIT (clone G2a; αTIGIT-G2a) or mIgG2a isotype control (cIg) twice a week from 2 weeks to 6 weeks posttransplant.

FIG. 2I shows competing risk analysis. (n = 10/group from 2 independent experiments). Data represent mean ± SEM. *p<0.05, ***p<0.001. Recipients were treated with 100 µg/mouse of anti-TIGIT (clone G2a; αTIGIT-G2a) or mIgG2a isotype control (cIg) twice a week from 2 weeks to 6 weeks posttransplant.

FIG. 3A shows a representative t-SNE analysis of PD-1, TIGIT, TIM-3, DNAM-1, CD101 and CD38 expression for MM-free and MM-bearing groups.

FIG. 3B shows frequency of DNAM-1⁺, TIM-3⁺, TIGIT⁺, PD-1⁺, DNAM-1-PD-1⁺ and CD101⁺CD38⁺ cells within CD8 T cells for MM-free and MM-bearing groups.

FIG. 3C shows frequency of IFNγ and TNF-expressing cells within CD8 T cells after PMA/ionomycin restimulation for MM-free and MM-bearing groups. (n = 11-12/group from two experiments; TNF and TIM-3 n = 3-6/group from 1 experiment).

FIG. 3D shows t-SNE analysis of PD-1, TIGIT, TIM-3, and DNAM-1 expression. MLL-AF9-bearing (AML-bearing) or naive (AML-free) mice were sacrificed 4 weeks posttransplant and BM was harvested to assess CD8 T cell phenotype.

FIG. 3E shows frequency of DNAM-1⁺, TIM-3⁺, TIGIT⁺, and DNAM- 1-PD-1⁺ cells within CD8 T cells for AML-free and AML-bearing groups.

FIG. 3F shows frequency of PD-1⁺ cells within CD8 T cells for AML-free and AML-bearing groups (top) and frequency of IFNy-expressing cells within CD8 T cells after PMA/ionomycin re-stimulation for AML-free and AML-bearing groups. (bottom) (n = 9/group from 2 experiments). Data represent mean ± SEM. Mann-Whitney U test or Student’s t-test were used for numerical values. * p<0.05, ** p<0.01, *** p<0.001.

FIG. 4A shows weighted nearest neighbor (WNN) embedding of combined ATAC and RNA data of CD8 cells colored by cluster (top) then annotated using CD8 T cell specific markers (bottom).

FIG. 4B shows CD4 T cells clustered and annotated in a manner analogous to FIG. 4A.

FIG. 4C shows embedding in FIG. 4A colored by experimental group (left). Centered and scaled cumulative gene expression (abbrev. ‘exp’) and gene accessibility (abbrev. ‘acc’, using gene activity score) of T_{EX} and T_{SCM} genes in CD8 T cells by experimental group (right). Wilcoxon Rank Sum test.

FIG. 4D shows embedding in FIG. 4B colored by experimental group (left). Centered and scaled cumulative gene expression (abbrev. ‘exp’) and gene accessibility of T_{EX} and T_{SCM} genes in CD4 T cells by experimental group (right). Wilcoxon Rank Sum test.

FIG. 4E shows gene accessibility scores of key cytokine receptor genes by experiment group in CD8 T cells.

FIG. 4F shows gene accessibility scores of key cytokine receptor genes by experiment group in CD4 T cells.

FIG. 4G shows a representative flow cytometry plot of PD-1 versus TOX expression in CD8 T cells.

FIG. 4H shows frequency of TOX⁺, PD-1⁺ and DNAM-1⁺ within CD8 T cells. (n = 7-10 /group from 2 independent experiments, TOX n = 3 - 5 /group from 1 experiment). Data represent mean ± SEM. One-way ANOVA with Tukey’s multiple comparisons test or Kruskal-Wallis test with Dunn’s multiple comparisons test. * p<0.05, ** p<0.01, ***p<0.001, ****p<0.0001.

FIG. 4I shows a representative flow cytometry plot of PD-1 versus TOX expression in CD4 conventional (FoxP3⁻) T cells.

FIG. 4J shows frequency of TOX⁺, PD-1⁺ and DNAM-1⁺ within CD4 conventional T cells. (n = 7-10 /group from 2 independent experiments, TOX n = 3 - 5 /group from 1 experiment). Data represent mean ± SEM. One-way ANOVA with Tukey’s multiple comparisons test or Kruskal-Wallis test with Dunn’s multiple comparisons test. * p<0.05, ** p<0.01, ***p<0.001, ****p<0.0001.

FIG. 5A shows MM-bearing recipients of T cell replete grafts were untreated (alloBMT) or administered 50 mg/kg cyclophosphamide on D+3 and D+4 (PT-Cy) and monitored for myeloma burden using M-band (left) (log G/A; n = 24 from 4 experiments; Mann-Whitney U test) and survival (right) (n = 17 from 3 experiments; Log-rank test).

FIG. 5B shows MM-bearing recipients of T cell deplete BM grafts (TCD) were treated as above and monitored for M-band (n = 5 from 1 experiment; Student’s t-test).

FIG. 5C shows representative contour plots.

FIG. 5D shows CD8, conventional CD4 (FoxP3⁻), and regulatory CD4 T cell enumeration per femur. Data represent mean ± SEM. One-way ANOVA with Tukey’s multiple comparisons test or Kruskal-Wallis test with Dunn’s multiple comparisons test. * p < 0.05 ** p < 0.01.

FIG. 5E shows naive T (T_{N}; CD44⁻CD62L⁻), central memory T (T_{CM}; CD44⁺CD62L⁺), effector memory T (T_{EM}; CD44⁺CD62L⁻), and effector T (T_{EFF}; CD44⁻CD62L⁻) within CD8 T cells. Data represent mean ± SEM. One-way ANOVA with Tukey’s multiple comparisons test or Kruskal-Wallis test with Dunn’s multiple comparisons test. * p < 0.05 ** p < 0.01 *** p <0.001.

FIG. 5F shows frequency of DNAM-1 and TIGIT expressing cells. Data represent mean ± SEM. One-way ANOVA with Tukey’s multiple comparisons test or Kruskal-Wallis test with Dunn’s multiple comparisons test. * p < 0.05 ** p < 0.01 *** p <0.001.

FIG. 5G shows TOX⁺, CD101⁺ and TIM-3⁺ cells. Data represent mean ± SEM. One-way ANOVA with Tukey’s multiple comparisons test or Kruskal-Wallis test with Dunn’s multiple comparisons test. ** p < 0.01 *** p <0.001.

FIG. 5H shows frequency of DNAM-1 and TIGIT expressing cells within conventional CD4 T cells. Data represent mean ± SEM. One-way ANOVA with Tukey’s multiple comparisons test or Kruskal-Wallis test with Dunn’s multiple comparisons test. * p < 0.05 ** p < 0.01 *** p <0.001.

FIG. 5I shows TOX⁺, CD101⁺ and TIM-3⁺ cells within conventional CD4 T cells. Data represent mean ± SEM. One-way ANOVA with Tukey’s multiple comparisons test or Kruskal-Wallis test with Dunn’s multiple comparisons test. * p < 0.05 ** p < 0.01.

FIG. 6A shows an example experimental design in which B6 MM-bearing recipients were transplanted with 5 × 10⁶ BM and 1 × 10⁶ CD8⁺ and 1 × 10⁶ CD4⁺ T cells from C3H.SW donors. Recipients were administered cyclophosphamide (50 mg/kg) on D+3 and D+4 and then either a vehicle control or immunotherapy from D+7 for 4 weeks.

FIG. 6B shows an M-band of recipients treated with rIgG2a (PT-Cy) or anti-PD-1 (PT-Cy + αPD-1). n = 10 from 2 experiments at 4 weeks; n = 5 from 1 experiment at 6 weeks.

FIG. 6C shows an M-band of recipients treated with rIgG2a (PT-Cy) or anti-TIM-3 (PT-Cy + αTIM-3). n = 5 from 1 experiment.

FIG. 6D shows M-band at 4 and 6 weeks post-alloBMT and GVHD clinical (including alloBMT mice not treated with PTCy; n = 5 from 1 experiment).

FIG. 6E shows IFNγ and TNF (pg/ml) in serum at D+10 and D+21 after alloBMT (n = 5 from 1 experiment).

FIG. 6F shows concatenated contour plots of TIGIT versus DNAM-1, and TIM-3 versus CD39 expression on CD8 T cells from BMA (representative of two experiments).

FIG. 6G shows concatenated density plots of NK cell frequency (Nkp46⁺ CD49b⁺) within white blood cells from BMA.

FIG. 6H shows myeloma total numbers per femur at week 6.

FIG. 6I shows T and NK cell total numbers per femur at week 6.

FIG. 6J shows number of DNAM-1⁺ and granzyme A⁺ perforin⁺ (GrzA⁺Pfp⁺) NK cells (n = 5/group from 1 experiment).

FIG. 6K shows results in which FlowSOM clustering was performed on concatenated CD4 T cells. Populations are colored based on expected anti-tumor properties. Green = activated effector or memory populations, orange = cytolytic, red = exhausted/suppressive and black = unknown.

FIG. 6L shows results in which FlowSOM clustering was performed on concatenated CD8 T cells at week 6 post-transplant. Heatmaps depict relative frequencies of populations across treatment groups. Populations are colored based on expected anti-tumor properties. Green = activated effector or memory populations, orange = cytolytic, red = exhausted/suppressive and black = unknown.

FIG. 6M shows frequency of DNAM-1⁻ and TIGIT-expressing cells within CD8⁺ T cells at week 6.

FIG. 6N shows frequency of CD39 and TIM-3 expressing cells within CD8 T cells at week 6.

FIG. 6O shows fold change in granzyme B (GrzB⁺) expression on CD8 T cells.

FIG. 6P shows total number of perforin-expressing (Pfp⁺; n = 5/group from 1 experiment) CD8 T cells at week 6 post-transplant. Data represent mean ± SEM. One-way ANOVA with Tukey’s multiple comparisons test or Kruskal-Wallis test with Dunn’s multiple comparisons test. * p < 0.05 ** p < 0.01 *** p <0.001.

FIG. 7A shows an example experimental design in which B6D2F1 recipients were lethally irradiated and transplanted with 5 × 10⁶ BM and 2 × 10⁶ T cells from HULK (IFN-γ-YFP × IL-10-GFP × FoxP3-RFP) donors and 1 × 10⁶ BCR-ABL-NUP98-HOXA9 leukemia cells. Recipients were untreated (haploBMT) or administered PT-Cy (50 mg/kg) on D+3 and D+4 (PT-Cy) with or without decoy-resistant IL-18 (PT-Cy + DR-18; 8 µg twice weekly from D+7 to week 5) or CD137 agonist antibody (PT-Cy + αCD137; 100 µg twice weekly from D+7 to week 5).

FIG. 7B shows the number of GFP⁺ leukemia cells in blood (left), leukemic death (middle), and overall median survival (right).

FIG. 7C shows total numbers of CD8⁺ T, CD4⁺ T, and NK cells. Mice with <5% leukemia cells in BM were euthanized at 21 days after transplantation, and BM was analyzed (n = 4 to 5; one experiment).

FIG. 7D shows percentage of IFN-γ-producing CD8⁺ and CD4⁺ T cells. Mice with <5% leukemia cells in BM were euthanized at 21 days after transplantation, and BM was analyzed (n = 4 to 5; one experiment).

FIG. 7E shows coexpression of DNAM-1 and TIGIT on CD8 T cells as representative contour plots (left) and quantified in individual mice (right). Mice with <5% leukemia cells in BM were euthanized at 21 days after transplantation, and BM was analyzed (n = 4 to 5; one experiment).

FIG. 7F shows MFI of TIGIT and DNAM-1 on CD8⁺ T cells. Mice with <5% leukemia cells in BM were euthanized at 21 days after transplantation, and BM was analyzed (n = 4 to 5; one experiment).

FIG. 7G shows expression of TIM-3 and TOX on CD8⁺ T cells as contour plots (left) and quantification of expression within CD8 T cells (right). Mice with <5% leukemia cells in BM were euthanized at 21 days after transplantation, and BM was analyzed (n = 4 to 5; one experiment).

FIG. 7H shows granzyme A and B expression in CD8⁺ T cells. Mice with <5% leukemia cells in BM were euthanized at 21 days after transplantation, and BM was analyzed (n = 4 to 5; one experiment).

FIG. 7I shows IFN-γ expression in NK cells, histogram (left) and quantification (right). Mice with <5% leukemia cells in BM were euthanized at 21 days after transplantation, and BM was analyzed (n = 4 to 5; one experiment).

FIG. 7J shows granzyme A and B production in NK cells, contour graph (top) and frequency of double positive cells within NK cells (bottom). One-way ANOVA with Tukey’s multiple comparisons test and log-rank for survival. Data are means ± SEM. *P < 0.05, **P < 0.01, and ***P < 0.001. Mice with <5% leukemia cells in BM were euthanized at 21 days after transplantation, and BM was analyzed (n = 4 to 5; one experiment).

FIG. 8A shows median survival. MM-bearing C57B⅙ recipients were transplanted with 5 x 10⁵ BM and 0.5 x 10⁶ CD4⁺ and 0.5 x 10⁶ CD8⁺ T cells from C3H.Sw donors. Recipients were treated with 100 µg/mouse of anti-TIGIT (clone G1; αTIGIT-G1) or cIg twice a week, from 3 weeks posttransplant for 6 weeks.

FIG. 8B shows log M-band at 6 and 8 weeks post-alloSCT. MM-bearing C57B⅙ recipients were transplanted with 5 x 10⁵ BM and 0.5 x 10⁶ CD4⁺ and 0.5 x 10⁶ CD8⁺ T cells from C3H.Sw donors. Recipients were treated with 100 µg/mouse of anti-TIGIT (clone G1; αTIGIT-G1) or cIg twice a week, from 3 weeks posttransplant for 6 weeks.

FIG. 8C shows clinical score. MM-bearing C57B⅙ recipients were transplanted with 5 x 10⁵ BM and 0.5 x 10⁶ CD4⁺ and 0.5 x 10⁶ CD8⁺ T cells from C3H.Sw donors. Recipients were treated with 100 µg/mouse of anti-TIGIT (clone G1; αTIGIT-G1) or cIg twice a week, from 3 weeks posttransplant for 6 weeks.

FIG. 8D shows competing risk analysis of GVHD- and myeloma-related mortality. n = 15/group from 2 experiments. Data represent mean ± SEM. Mann-Whitney U test was used for numerical values. Log-rank test for survival. MM-bearing C57B⅙ recipients were transplanted with 5 x 10⁵ BM and 0.5 x 10⁶ CD4⁺ and 0.5 x 10⁶ CD8⁺ T cells from C3H.Sw donors. Recipients were treated with 100 µg/mouse of anti-TIGIT (clone G1; αTIGIT-G1) or cIg twice a week, from 3 weeks posttransplant for 6 weeks.

FIG. 9 shows a heatmap of differentially expressed genes in CD8 T cell clusters. B6 recipients were transplanted with 5 x 10⁶ BM with 0.5 x 10⁶ CD4 + 0.5 x 10⁶ CD8 T cells from C3H.SW donors (alloBMT) and some recipients were treated with 50 mg/kg cyclophosphamide on D+3 and D+4 after transplantation (PTCy). Mice were sacrificed 14 days after transplant and BM was harvested. CD8 and CD4 T cells were sort purified from BM of alloBMT and PT-Cy recipients and nuclei were processed for 10x genomics multiome sequencing. Heatmap of top 20 differentially expressed genes in each cluster. Clusters were based on WNN embedding of combined ATAC and RNA data of CD8 cells.

FIG. 10 shows a heatmap of differentially expressed genes in CD4 T cell clusters. B6 recipients were transplanted with 5 x 10⁶ BM with 0.5 x 10⁶ CD4 + 0.5 x 10⁶ CD8 T cells from C3H.SW donors (alloBMT) and some recipients were treated with 50 mg/kg cyclophosphamide on D+3 and D+4 after transplantation (PTCy). Mice were sacrificed 14 days after transplant and BM was harvested. CD8 and CD4 T cells were sort purified from BM of alloBMT and PT-Cy recipients and nuclei were processed for 10x genomics multiome sequencing. Heatmap of top 20 differentially expressed genes in each cluster. Clusters were based on WNN embedding of combined ATAC and RNA data of CD4 cells.

FIG. 11 shows a heatmap of differentially expressed genes in CD8 T cells from alloBMT versus PT-Cy recipients. B6 recipients were transplanted with 5 x 10⁶ BM with 0.5 x 10⁶ CD4 + 0.5 x 10⁶ CD8 T cells from C3H.SW donors (alloBMT) and some recipients were treated with 50 mg/kg cyclophosphamide on D+3 and D+4 after transplantation (PTCy). Mice were sacrificed 14 days after transplant and BM was harvested. CD8 and CD4 T cells were sort purified from BM of alloBMT and PT-Cy recipients and nuclei were processed for 10x genomics multiome sequencing. Heatmap of top 50 differentially expressed genes across treatment groups.

FIG. 12 shows a heatmap of differentially expressed genes in CD4 T cells from alloBMT versus PT-Cy recipients. B6 recipients were transplanted with 5 x 10⁶ BM with 0.5 x 10⁶ CD4 + 0.5 x 10⁶ CD8 T cells from C3H.SW donors (alloBMT) and some recipients were treated with 50 mg/kg cyclophosphamide on D+3 and D+4 after transplantation (PTCy). Mice were sacrificed 14 days after transplant and BM was harvested. CD8 and CD4 T cells were sort purified from BM of alloBMT and PT-Cy recipients and nuclei were processed for 10x genomics multiome sequencing. Heatmap of top 50 differentially expressed genes across treatment groups.

FIG. 13A shows a heatmap of motif activity score in CD8 T cells from alloBMT versus PT-Cy recipients. B6 recipients were transplanted with grafts from C3H.SW donors as described. Recipients were untreated (alloBMT) or administered PTCy. Mice were sacrificed at D+14 and BM was harvested. CD8 and CD4 T cells were sort purified from BM of alloBMT and PT-Cy recipients and nuclei were processed for 10x genomics multiome sequencing. Heatmap of motif activity scores across individual cells in CD8 T cells across treatment groups.

FIG. 13B shows a heatmap of motif activity score in CD4 T cells from alloBMT versus PT-Cy recipients. B6 recipients were transplanted with 5 x 10⁶ BM with 0.5 x 10⁶ CD4 + 0.5 x 10⁶ CD8 T cells from C3H.SW donors (alloBMT) or B6 donors (synBMT). Some alloBMT recipients were treated with 50 mg/kg cyclophosphamide on D+3 and D+4 after transplantation (alloBMT + PTCy). Mice were sacrificed 14 days after transplant and BM was harvested. Heatmap of motif activity scores across individual cells in CD4 T cells across treatment groups.

FIG. 14A shows representative flow cytometry plots of TIGIT versus DNAM-1 expression in CD8 T cells (top), and frequency of TIGIT⁺ (lower left), TIM-3⁺ (lower middle), and granzyme B⁺ (GrzB) (lower right) cells within CD8 T cells.

FIG. 14B shows representative flow cytometry plots of TIGIT versus DNAM-1 expression in conventional (FoxP3⁻) CD4 T cells (top), and frequency of TIGIT⁺ (lower left), TIM-3⁺ (lower middle), and granzyme B⁺ (GrzB) (lower right) cells within conventional CD4 T cells. (n = 7-10 /group from 2 independent experiments, TOX n = 3 -5 /group from 1 experiment). Data represent mean ± SEM. One-way ANOVA with Tukey’s multiple comparisons test or Kruskal-Wallis test with Dunn’s multiple comparisons test. * p<0.05, ** p<0.01, ***p<0.001.

FIG. 15A shows PT-Cy reduces the total number of CD8 and CD4 T cells in the bone marrow. C57B⅙ recipients were transplanted with 5 x 10⁵ BM and 1 x 10⁶ CD4⁺ and 1 x 10⁶ CD8⁺ T cells from C3H.Sw donors. Recipients were treated with PBS (alloBMT) or 50 mg/kg cyclophosphamide (alloBMT + PT-Cy) on D+3 and D+4 post-transplant. The total number of CD8 and CD4 T cells in BM was calculated at D+14 in MM-free mice (n = 5/group). Data represent mean ± SEM. Unpaired t test was used for numerical values. **p<0.01, ***p<0.001.

FIG. 15B shows PT-Cy reduces the total number of CD8 and CD4 T cells in the bone marrow per the procedure for FIG. 15A. The total number of CD8 and CD4 T cells in BM was calculated at D+21 in MM-bearing mice (n = 9/group from two experiments). Data represent mean ± SEM. Unpaired t test was used for numerical values. ***p<0.001.

FIG. 16A shows anti-CD137 promoted CD8 T cell differentiation and activation in blood. MM-bearing C57B⅙ recipients were transplanted with 5 x 10⁵ BM and 1 x 10⁶ CD4⁺ and 1 x 10⁶ CD8⁺ T cells from C3H.sW donors. Recipients were administered 50 mg/kg PTCy on D+3 and D+4 and then either a vehicle control or 8 µg/dose DR-18, or 100 µg/dose anti- CD137 from D+7 for 4 weeks. Mice were sacrificed at 6 weeks after alloBMT and blood was harvested for analysis with flow cytometry. FIG. 16A shows total number of CD8 T, CD4 T and NK cells. For the figure, n = 10 - 12/group from 2 experiments; for NK Pfp and GrzA, n = 5/group from 1 experiment. Data is mean ± SEM. *p<0.05, **p<0.001, ***p<0.0001.

FIG. 16B shows anti-CD137 promoted CD8 T cell differentiation and activation in blood per the procedure of FIG. 16A. FIG. 16B shows expression of granzyme A (GrzA), perforin (Pfp), DNAM-1, and CD39 on blood NK cells. For the figure, n = 10 - 12/group from 2 experiments; for NK Pfp and GrzA, n = 5/group from 1 experiment. Data is mean ± SEM. *p<0.05, **p<0.001, ***p<0.0001.

FIG. 16C shows anti-CD137 promoted CD8 T cell differentiation and activation in blood per the procedure of FIG. 16A. FIG. 16C shows differentiation based on CD44 and CD62L expression (T_{N}: CD44⁻CD62L⁺; T_{CM}: CD44⁺CD62L⁺; T_{EM}: CD44⁺CD62L⁻; T_{EFF}: CD44⁻ CD62L⁻) of conventional CD4 T cells. For the figure, n = 10 - 12/group from 2 experiments; for NK Pfp and GrzA, n = 5/group from 1 experiment. Data is mean ± SEM. *p<0.05, **p<0.001, ***p<0.0001.

FIG. 16D shows anti-CD137 promoted CD8 T cell differentiation and activation in blood per the procedure of FIG. 16A. FIG. 16D shows differentiation based on CD44 and CD62L expression (T_{N}: CD44⁻CD62L⁺; T_{CM}: CD44⁺CD62L⁺; T_{EM}: CD44⁺CD62L⁻; T_{EFF}: CD44⁻ CD62L⁻) of CD8 T cells. For the figure, n = 10 - 12/group from 2 experiments; for NK Pfp and GrzA, n = 5/group from 1 experiment. Data is mean ± SEM. *p<0.05, **p<0.001, ***p<0.0001.

FIG. 16E shows anti-CD137 promoted CD8 T cell differentiation and activation in blood per the procedure of FIG. 16A. FIG. 16E shows frequency of granzyme B⁺, PD-1⁺, CD39+ or TOX⁺ cells within CD8 T cells. For the figure, n = 10 - 12/group from 2 experiments; for NK Pfp and GrzA, n = 5/group from 1 experiment. Data is mean ± SEM. *p<0.05, **p<0.001, ***p<0.0001.

FIG. 17A shows FlowSOM clustering of CD4 and CD8 T cells reveals distinct populations after agonist immunotherapy, and DR-18 expanded CD62L negative Tregs while CD137 expanded effector CD4 T cells after PT-Cy. MM-bearing C57B⅙ recipients were transplanted with 5 x 10⁵ BM and 1 x 10⁶ CD4⁺ and 1 x 10⁶ CD8⁺ T cells from C3H.sW donors. Recipients were administered 50 mg/kg PTCy on D+3 and D+4 and then either a vehicle control or 8 µg/dose DR-18, or 100 µg/dose anti-CD137 from D+7 for 4 weeks. Mice were sacrificed at 6 weeks after transplant and BM was harvested for analysis with flow cytometry and FlowSOM clustering using mean fluorescence intensity (MFI). FIG. 17A shows a heatmap of marker MFI in each FlowSOM cluster from concatenated CD4 T cells from all three treatment groups.

FIG. 17B shows DR-18 expanded CD62L negative Tregs while CD137 expanded effector CD4 T cells after PT-Cy per the procedure of FIG. 17A. FIG. 17B shows enumeration of key CD4 T cell clusters. n = 5 - 7/group from 1 experiment; data is mean ± SEM.

FIG. 17C shows FlowSOM clustering of CD8 T cells reveals distinct populations after agonist immunotherapy. MM-bearing C57B⅙ recipients were transplanted with 5 x 10⁵ BM and 1 x 10⁶ CD4⁺ and 1 x 10⁶ CD8⁺ T cells from C3H.SW donors. Recipients were administered 50 mg/kg PTCy on D+3 and D+4 and then either a vehicle control or 8 µg/dose DR-18, or 100 µg/dose anti-CD137 from D+7 for 4 weeks. Mice were sacrificed at 6 weeks after transplant and BM was harvested for analysis with flow cytometry and FlowSOM clustering using mean fluorescence intensity (MFI). Heatmap depicts marker MFI in each FlowSOM cluster from CD8 T cells. n = 5 - 7 /group from 1 experiment.

FIG. 18A shows AML relapse after PT-Cy drives CD8 T cell exhaustion. MLL-AF9-bearing (AMLbearing) or AML-free mice were sacrificed 3 weeks post-transplant. All recipients received 50 mg/kg PT-Cy on D+3 and D+4. Flow cytometry plot of TIM-3 and CX3CR1 on CD8 T cells. (n = 8-9/group from 1 experiment). Data represent mean ± SEM. Student’s t-test was used for numerical values. * p<0.05.

FIG. 18B shows AML relapse after PT-Cy drives CD8 T cell exhaustion per the procedure of FIG. 18A. FIG. 18B shows enumeration of the frequency of TIM-3⁺ CX3CR1⁻ cells within CD8 T cells. (n = 8-9/group from 1 experiment). Data represent mean ± SEM. Student’s t-test was used for numerical values. * p<0.05.

FIG. 19 shows human T_{SCM} express the IL-18R receptor after allogeneic stem cell transplantation. Concatenated IL-18R expression data combined from 6 patients at day +60 after allogeneic stem cell transplantation. Tₙₐᵢᵥₑ cells were CD45RA⁺ CCR7⁺ CD95⁻ and T_{SCM} cells were CD45RA⁺ CCR7⁺ CD95⁺ CD28⁺. CD4 conventional T cells (CD4 T_{con}) were analyzed after regulatory T cells were excluded (CD25hi CD127⁻). Dotted line indicates background staining level in fluorescence minus one (FMO) control on all CD45RA⁺ CCR7⁺ cells.

FIG. 20A shows DR-18 expanded CD62L negative Tregs while CD137 expanded effector CD4 T cells after PT-Cy per the procedure of FIG. 17A. FIG. 20A shows representative flow cytometry plots (n = 10 - 14 / group from 2 experiments). *p<0.05, **p<0.001, ***p<0.0001.

FIG. 20B shows DR-18 expanded CD62L negative Tregs while CD137 expanded effector CD4 T cells after PT-Cy per the procedure of FIG. 17A. FIG. 20B shows frequency of CD6L-CD69+ Tregs (n = 10 - 14 / group from 2 experiments). *p<0.05, **p<0.001, ***p<0.0001.

### DETAILED DESCRIPTION

The disclosure provides improved methods for treating hematological malignancies (e.g., myeloma, leukemia) that are treatable with tissue (e.g., bone marrow, blood stem cell) transplants. The methods involve enrichment of a population of donor T cells for a stem-like memory T cell phenotype and agonist immunotherapy to enhance the graft-versus-tumor (GVT) response. The methods are effective for treatment of malignancies that are otherwise resistant to previous tissue transplant methods.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

The phrase “pharmaceutically acceptable carrier” is art recognized and includes a pharmaceutically acceptable material, composition, or vehicle, suitable for administering compounds used in the methods described herein to subjects, e.g., mammal subjects or human subjects. The methods herein can include administration of one or more agents that are formulated with one or more pharmaceutically acceptable carriers to a subject.

The language “therapeutically effective amount” or a “therapeutically effective dose” of a compound is the amount necessary to or sufficient to provide a detectable improvement of at least one cause of and/or symptom associated with or caused by the condition, disease, or disorder being treated. The therapeutically effective amount can be administered as a single dose or in multiple doses over time. Two or more compounds can be used together to provide a “therapeutically effective amount” to provide a detectable improvement wherein the same amount of either compound alone would be insufficient to provide a therapeutically effective amount. “Therapeutically effective amount,” as used herein refers to an amount of an agent which is effective, upon single or multiple dose administration to the cell or subject, decreasing at least one sign or symptom of the disease or disorder, or prolonging the survivability of the patient with such a disease or disorder beyond that expected in the absence of such treatment.

An agent can be administered to a subject, either alone or in combination with one or more therapeutic agents, as a pharmaceutical composition in mixture with conventional excipient, e.g., pharmaceutically acceptable carrier. The agent can be administered using any suitable method or mode of administration, including but not limited to parenteral administration, injection, intravascular injection, intravenous injection, infusion (e.g., using a pump), and the like. A composition can be administered according to any suitable regimen, for example, once a day, once a week, every two weeks, once a month, or more or less frequently, depending on the specific needs of the subject to be treated. The specific pharmacokinetic and pharmacodynamic properties of the composition to be administered can affect dosing. Such administration can be used as a chronic or acute therapy. The amount of active ingredient that can be combined with the carrier materials to produce a single dosage form can vary depending upon the host treated and the particular mode of administration.

Cells and/or subjects can be treated and/or contacted with one or more standard cancer therapeutic treatments, including but not limited to surgery, chemotherapy, radiotherapy, gene therapy, immune therapy, anti-angiogenic therapy, hormonal therapy, tissue transplant, blood transplant, bone marrow transplant, and/or another therapy or treatment, for example, as may be prescribed by a health care provider.

A “decoy resistant” IL-18 (DR-18) agent is a variant, mutant, or mimic of interleukin-18 (IL-18) that binds to IL-18 receptor (IL-18R), thereby inducing/enhancing/stimulating IL-18 signaling activity, but exhibits little to no binding to the inhibitory IL-18 binding protein (IL-18BP). DR-18 agents are therefore IL-18R agonists that are resistant to inhibition by IL-18BP. Examples of DR-18 agents, and of methods of making and using DR-18 agents, are described in U.S. Pat. App. Pub. No. 2019/0070262 A1 and U.S. Pat. App. Pub. No. 2021/0015891 A1, both of which are incorporated herein by reference in their entirety. A decoy-resistant IL-18 (DR-18) treatment includes any treatment that includes administration of one or more DR-18 agents to a subject.

### METHODS FOR TREATING HEMATOLOGICAL MALIGNANCIES

Tissue transplant for treatment of cancers and malignancies can be ineffective for conditions that are not sensitive to graft-versus-tumor (GVT) effects of immune cells of the transplanted tissue. For example, allogeneic bone marrow transplant for treatment of myeloma is potentially promising but is often ineffective due to myeloma being largely resistant to GVT effects, and the procedure can increase risk to the patient due to immunosuppression and graft-versus-host disease (GVHD). In at least some instances, patients with other malignancies, such as acute myeloid leukemia (AML), have reduced GVT sensitivity as well compared to other more highly sensitive malignancies, such as chronic myeloid leukemia (CML), which may be more tractable for these treatments. A better understanding of the differential responses of different malignancies to these treatments can enable the implementation of improved treatments that enhance the GVT effect without causing additional detrimental effects to the patient, e.g., in the form of increased GVHD frequency or severity.

The disclosure provides methods for treating hematological malignancies by modulating immune cells, such as T cells and/or NK cells, to enrich for stem-like phenotypes in the immune cells and stimulating the modulated immune cells to enhance a GVT response. While the modulated immune cells can be enriched for a stem-like phenotype by any suitable method, in example aspects and embodiments disclosed herein, this enrichment can occur with depletion of exhausted alloreactive donor T cells which can reduce the immune response against host tissues by the donor T cell population. This depletion can spare donor T cells that have a stem-like memory phenotype. The stem-like memory T cells can be stimulated with one or more agonist immunotherapies to strongly enhance a GVT response of the T cells in any of various hematological malignancies, including but not limited to myeloma and leukemia, without exacerbation of GVHD.

Accordingly, in various aspects, the disclosure provides a method for treating a hematological malignancy in a subject, the method comprising: transplanting a tissue that comprises a plurality of T cells to the subject and stimulating the plurality of T cells to enhance a GVT response of the plurality of T cells to treat the hematological malignancy. In at least some embodiments, the method further comprises enriching for a stem-like memory T cell phenotype in the plurality of T cells. By enriching or selecting for a stem-like memory T cell phenotype in the plurality of T cells, the reactivity of donor T cells to host tissue can be reduced. Donor immune cells can be treated with an agonist immunotherapy to stimulate the immune cells and enhance the GVT response for treating various malignancies, including myeloma and leukemia, as well as lowering the risk of GVHD and relapse of disease for cancers and malignancies that are treatable at least in part by tissue transplantation.

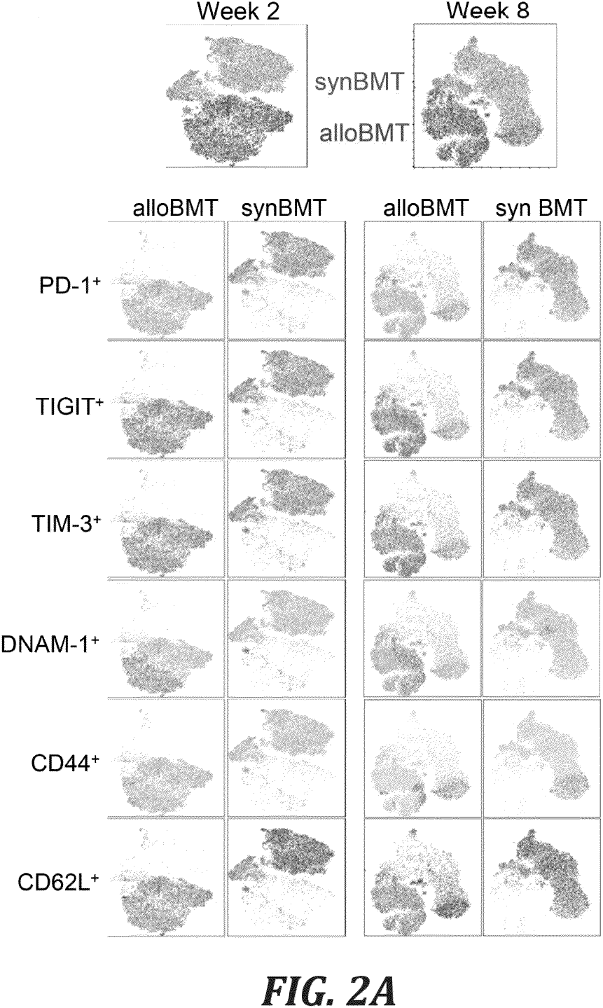

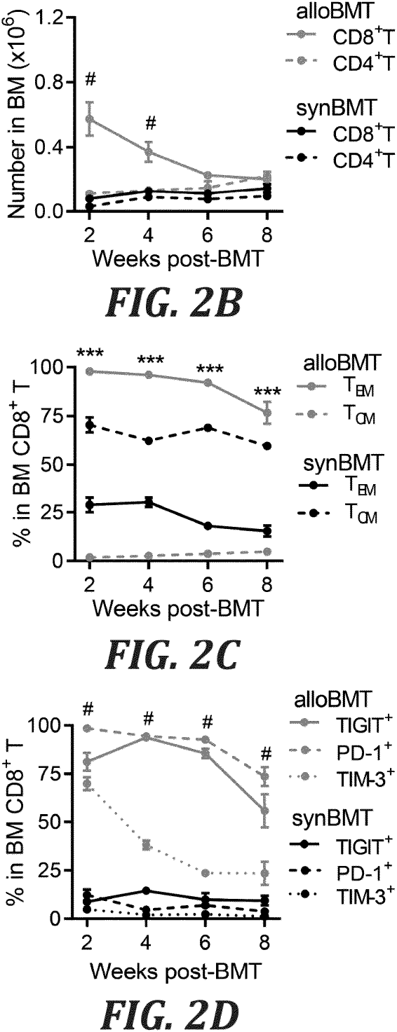

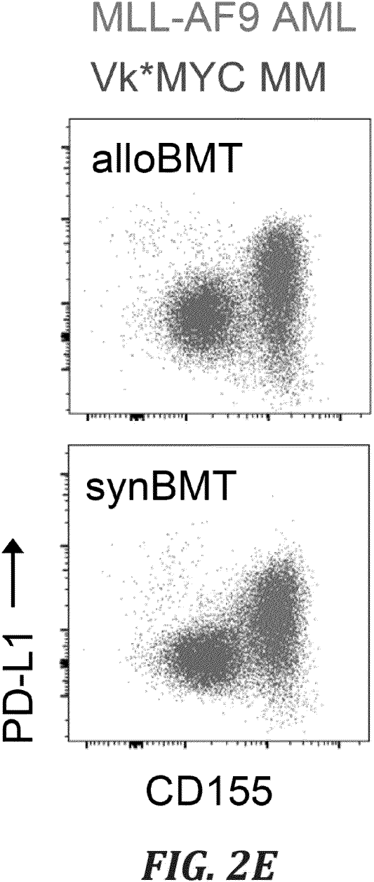

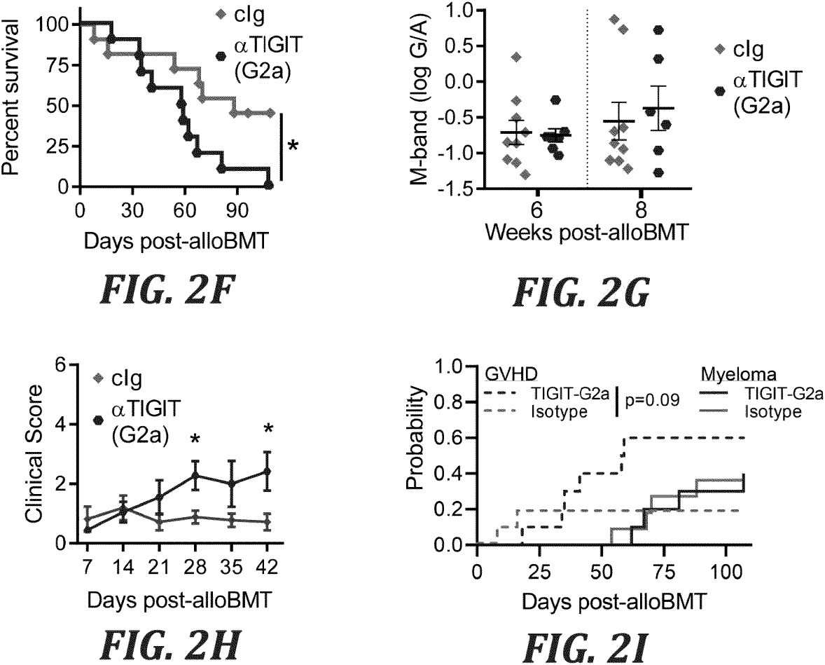

In embodiments, the hematological malignancy includes myeloma and/or leukemia. In at least some embodiments, the tissue being transplanted includes bone marrow and/or blood stem cells and can be allogeneic to the subject. In addition, in various embodiments, subjects treatable with the methods disclosed herein can have a higher probability of GVHD and/or a higher risk of relapse of the hematological malignancy, and the method can reduce the probability of GVHD and/or the risk of relapse. For example, in the case of allogeneic bone marrow transplantation (alloBMT), donor CD8⁺ T cells, particularly effector memory T cells (T_{EM}), become significantly expanded and exhausted due to responses to allogeneic antigens, rather than tumor antigens, in myeloma (FIG. 2A, FIG. 2B, FIG. 2C, FIG. 2D). In at least some instances, subversion of a graft-versus myeloma (GVM) response can be due to donor CD8⁺ T cells expressing high levels of TIGIT⁺ and PD-1⁺ in response to alloantigen and being inactivated via interaction with cognate inhibitory receptor ligands expressed by myeloma (FIG. 2E). As described herein, TIGIT blockade exacerbated GVHD without promoting GVM (FIG. 2F, FIG. 2G, FIG. 2H, FIG. 2I), the main driver of T cell exhaustion in the alloBMT setting is alloantigen rather than tumor-specific antigen, potentially explaining the heightened risk of GVHD and malignancy relapse in at least some myeloma patient groups. By depleting exhausted alloreactive immune cells from the donor immune cell population, the clinician can target stem-like memory T cells remaining in the donor cells to enhance tumor-specific immunity without increasing risk of GVHD. In this manner, the methods disclosed herein enable improved treatment of malignancies with therapies that involve tissue transplantation.

In a general sense, enriching the donor immune cells for a stem-like memory T cell phenotype involves increasing a frequency, a prevalence, and/or an availability of a desired phenotype within the donor immune cells. While this can potentially be achieved with other approaches, in example embodiments disclosed herein, this enrichment can comprise actively depleting at least a portion of the plurality of T cells. For example, an alloreactive portion of the T cells can be preferentially depleted relative to a stem-like memory T cell portion of the T cells, thereby enriching for the stem-like memory T cell portion in the overall population of T cells. This strategy can be beneficial as it can utilize an existing or a modified version of an existing treatment, such as a post-transplant cyclophosphamide (PT-Cy) treatment, which can be administered to the subject and/or contacted to the donor immune cells for the beneficial and unexpected result of effectively enriching for the stem-like memory T cell phenotype in the donor immune cells.

Accordingly, in another aspect, the disclosure provides a method for treating a hematological malignancy in a subject, the method comprising: transplanting a tissue that comprises a plurality of T cells to the subject, administering a PT-Cy treatment to the subject to deplete at least a portion of the plurality of T cells and enrich for a stem-like memory T cell phenotype in the plurality of T cells, and administering an agonist immunotherapy to the subject to enhance a GVT response of the plurality of T cells to treat the hematological malignancy.

In various embodiments, a method of the disclosure comprises administering a therapeutically effective amount of cyclophosphamide to a subject after transplant of one or more tissues to the subject (PT-Cy treatment) for enrichment of the stem-like memory T cell phenotype in the donor immune cells. The dose of cyclophosphamide used in a particular cyclophosphamide administration, and the frequency and extent of administration for a particular PT-Cy treatment, can vary as needed to achieve a desired outcome. The PT-Cy treatment can be administered alone or in combination with one or more standard cancer therapeutic treatments, including but not limited to surgery, chemotherapy, radiotherapy, gene therapy, immune therapy, anti-angiogenic therapy, hormonal therapy, tissue transplant, blood transplant, bone marrow transplant, and/or another therapy or treatment, for example, as may be prescribed by a health care provider.

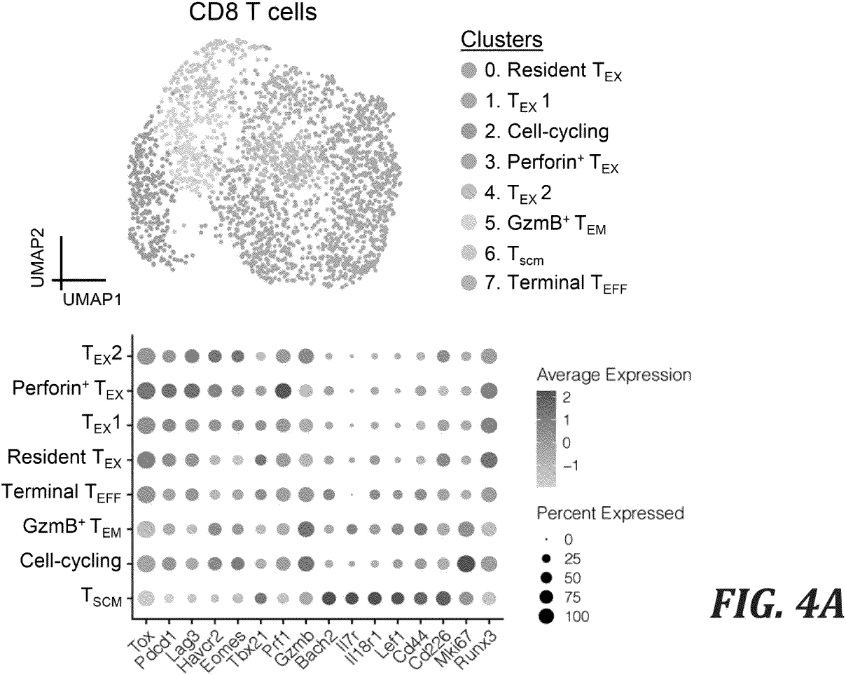

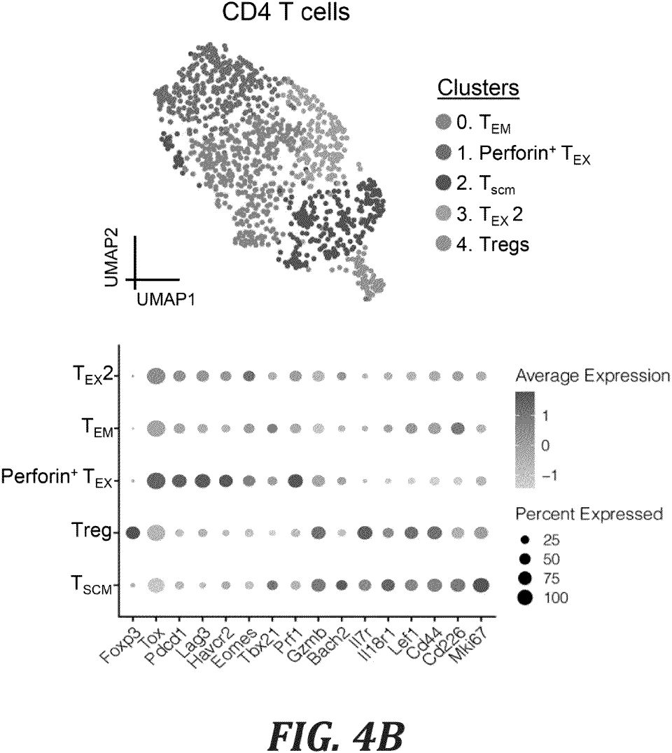

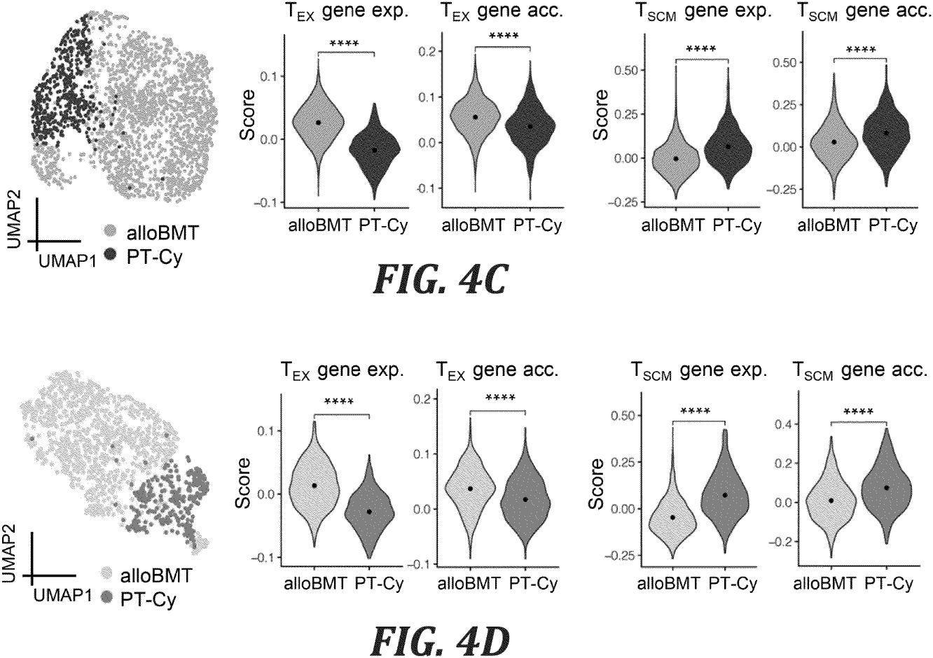

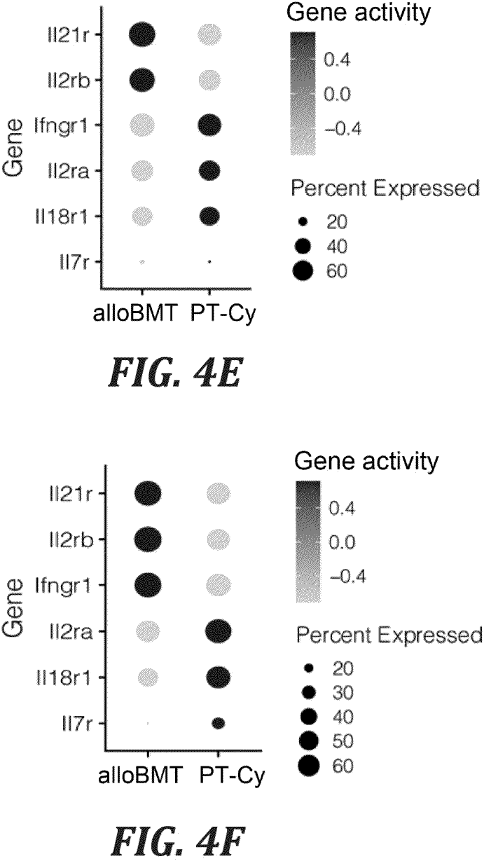

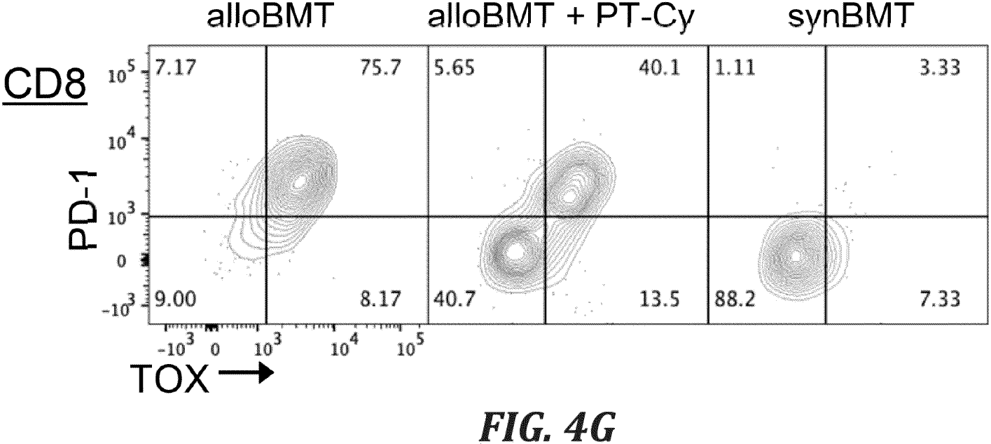

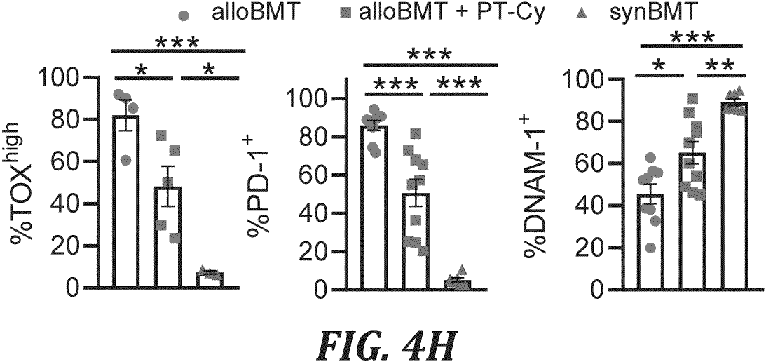

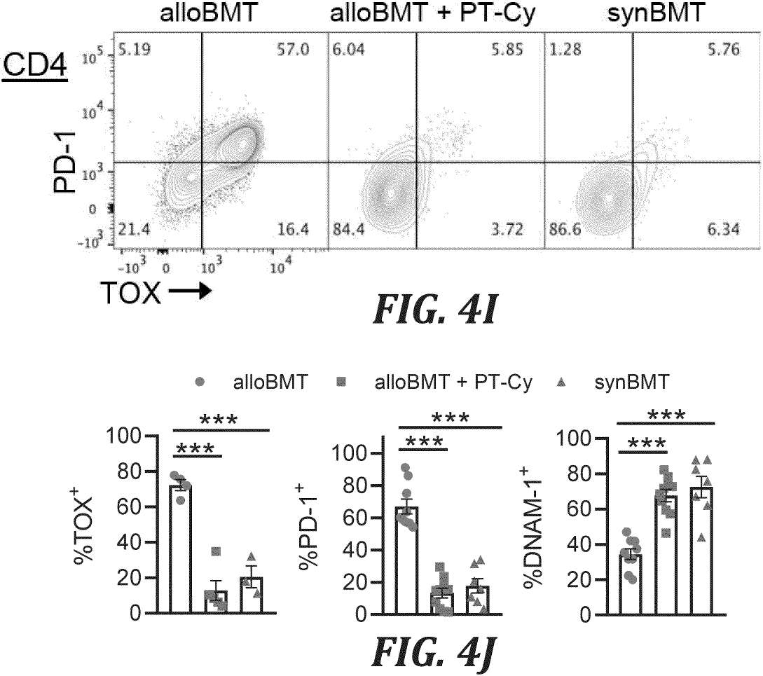

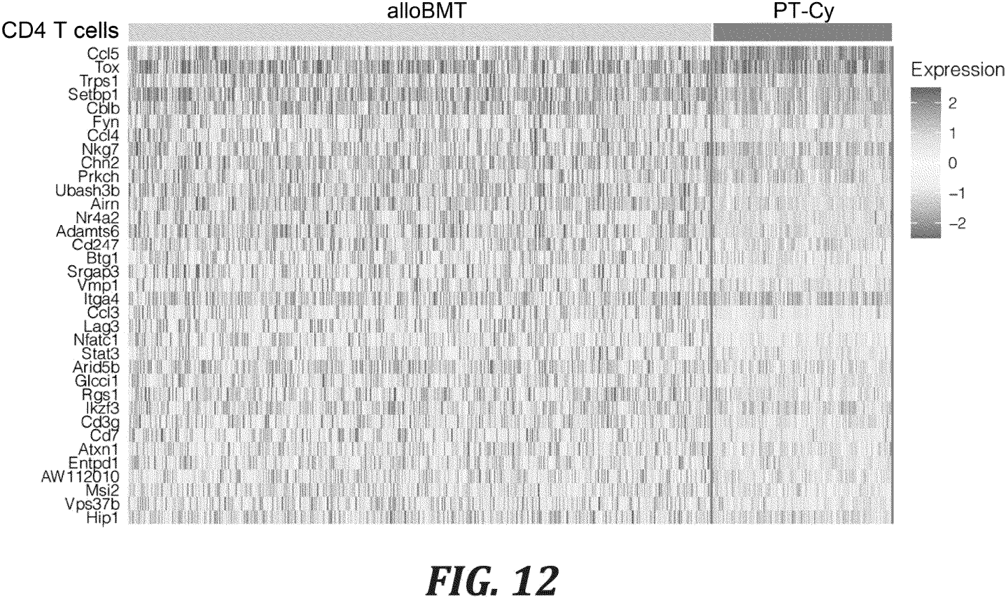

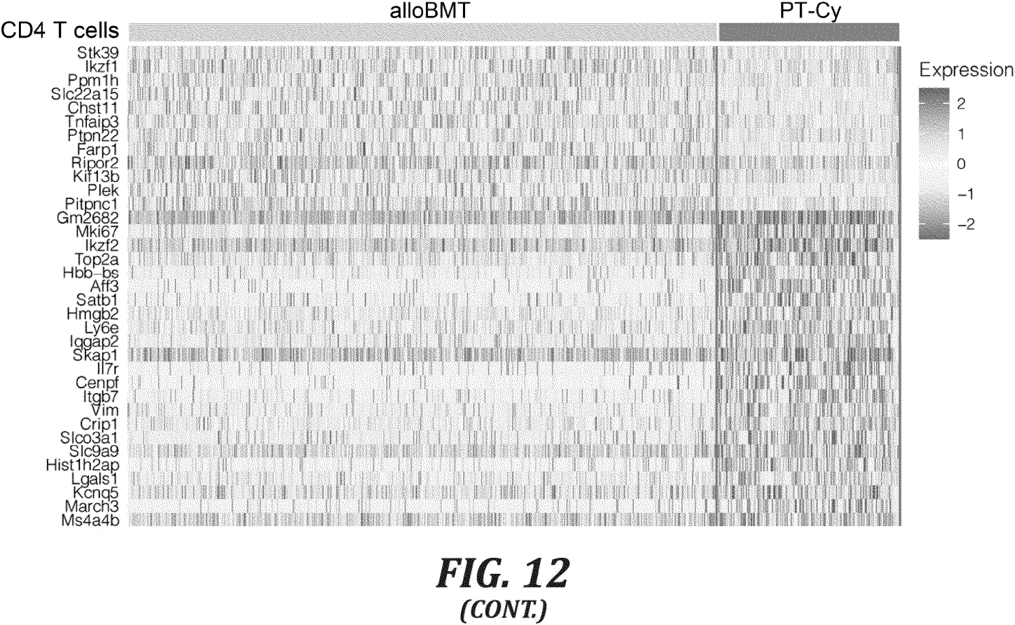

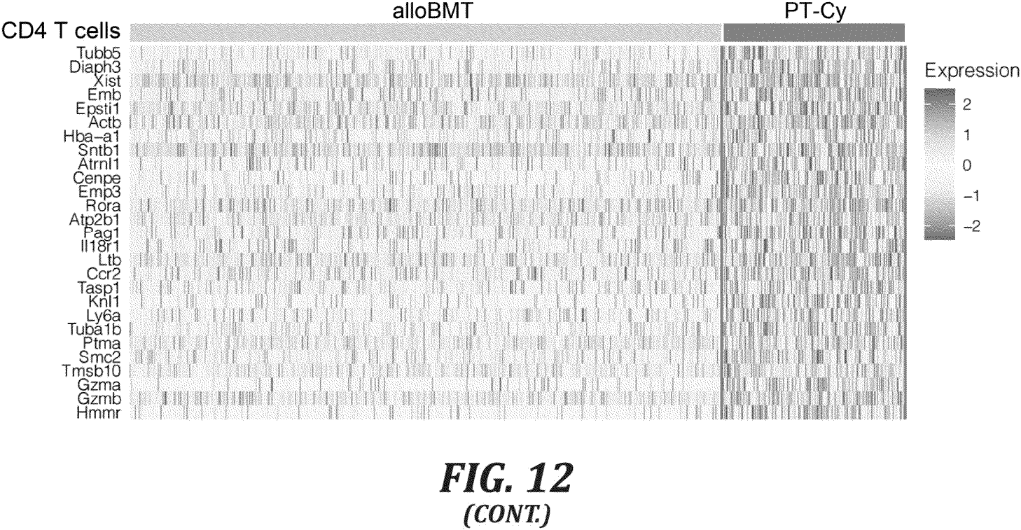

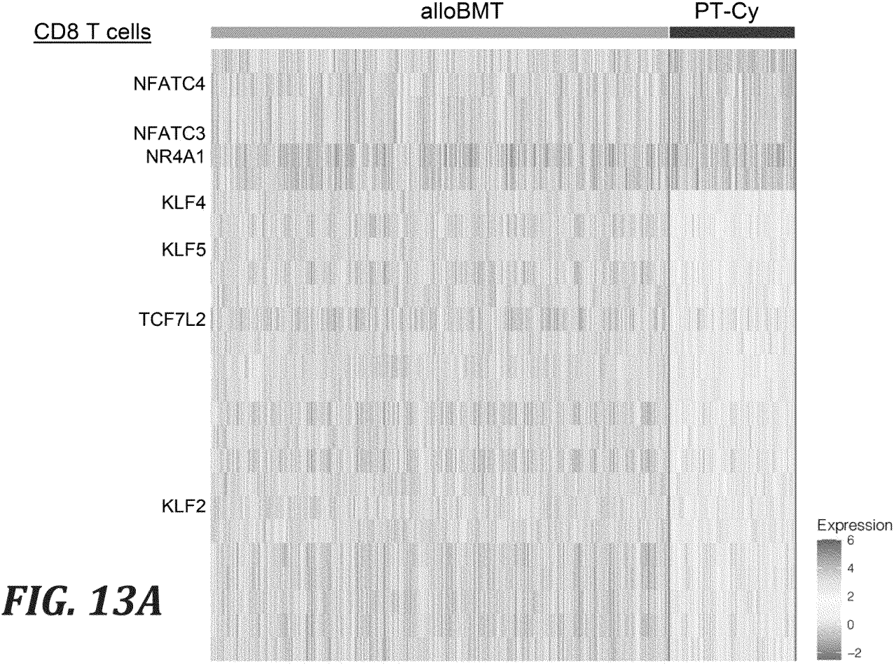

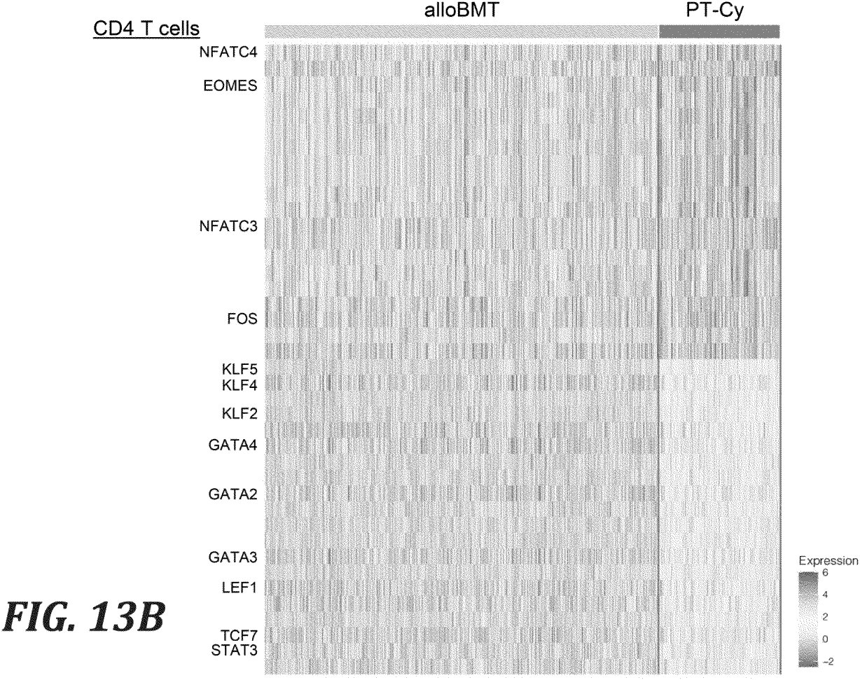

The prevalence of the desired stem-like memory T cell phenotype in the donor immune cell population, e.g., after administering the PT-Cy treatment, can be evaluated with molecular biology techniques as described herein or as known in the art. While other features may be present in a particular stem-like memory T cell phenotype, in at least some embodiments, the stem-like memory T cell phenotype or signature can include an increased chromatin accessibility in a cytokine signaling gene (FIG. 4E, and FIG. 4F), and/or an increased expression of interleukin-18 receptor (IL-18R), Transcription Factor 7 (TCF7), Transcription Factor 7 Like 2 (TCFL2), Krüppel-like transcription factor 2 (KLF2), Krüppel-like transcription factor 4 (KLF4), and/or Krüppel-like transcription factor 5 (KLF5) by at least a portion of the plurality of T cells, e.g., a stem-like memory T cell portion of the T cells (FIG. 12, FIG. 13A, and FIG. 13B). The stem-like memory T cells can include an increase in CD8⁺ central memory (T_{CM}; CD44⁺CD62L⁺) and a decrease in terminal effector (T_{EFF}; CD44⁻CD62L⁻) T cells. In at least some instances, the PT-Cy treatment effectively eliminates alloantigen-driven CD8⁺ T cell exhaustion and enables exhaustion to instead be driven by myeloma as a result of a GVT response. As would be understood by the person having ordinary skill in the art, additional features can be present in the phenotype of the stem-like memory T cells without departing from the scope of the disclosure.

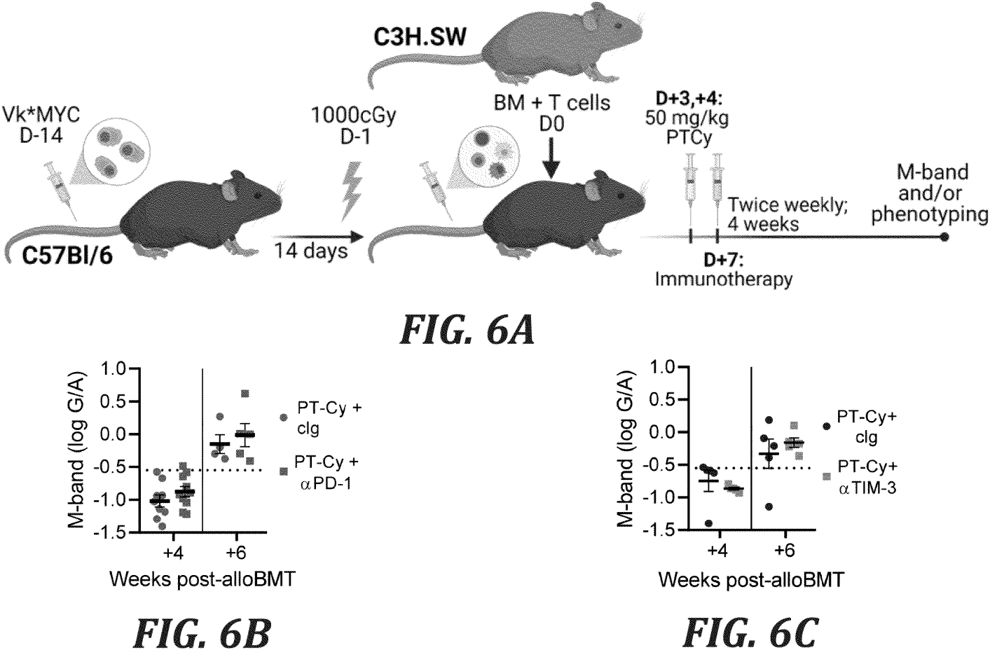

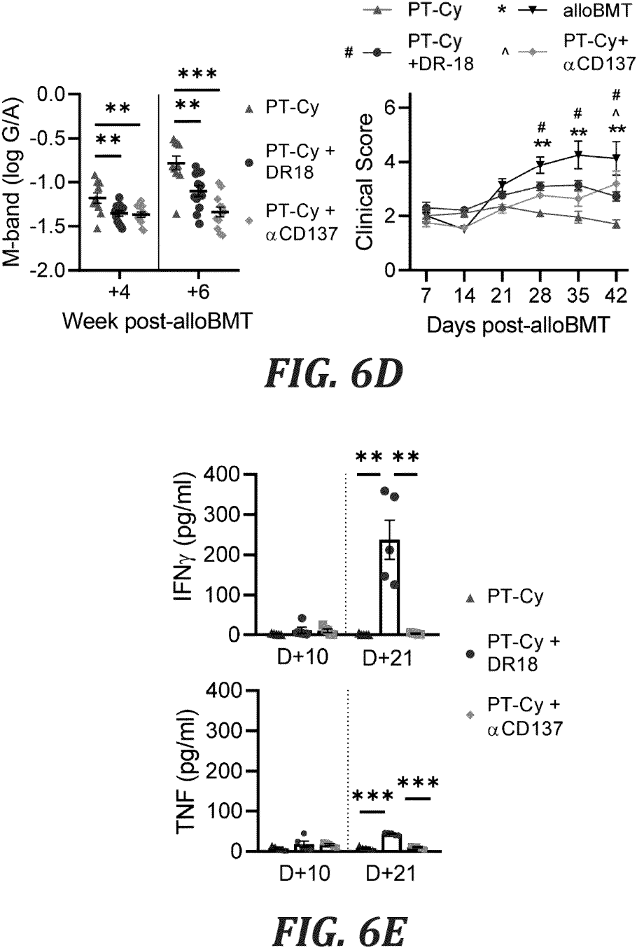

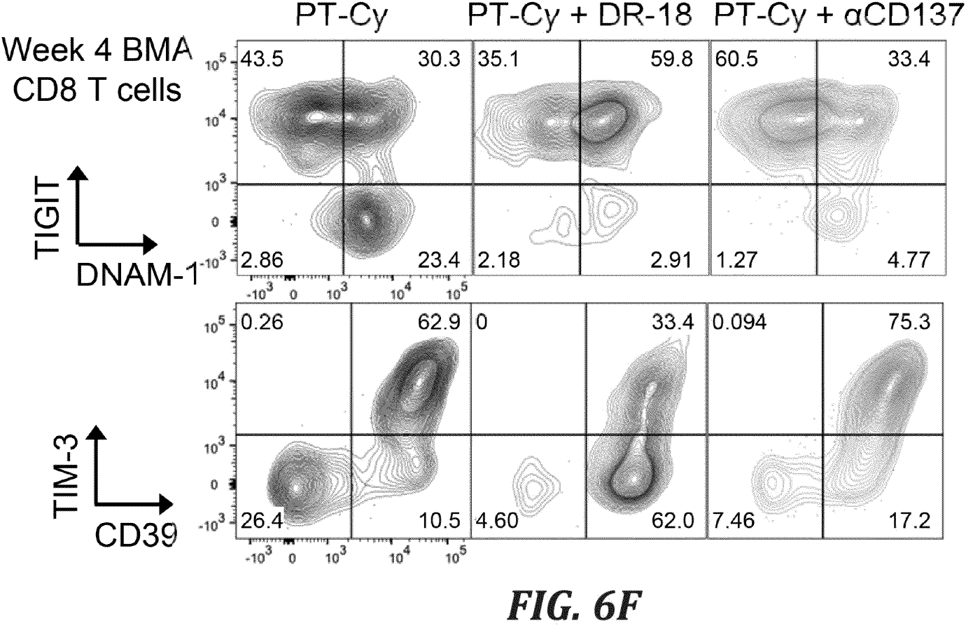

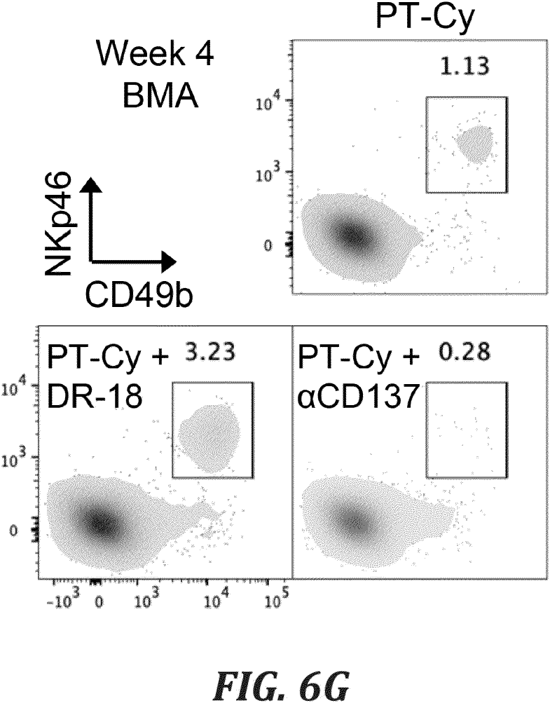

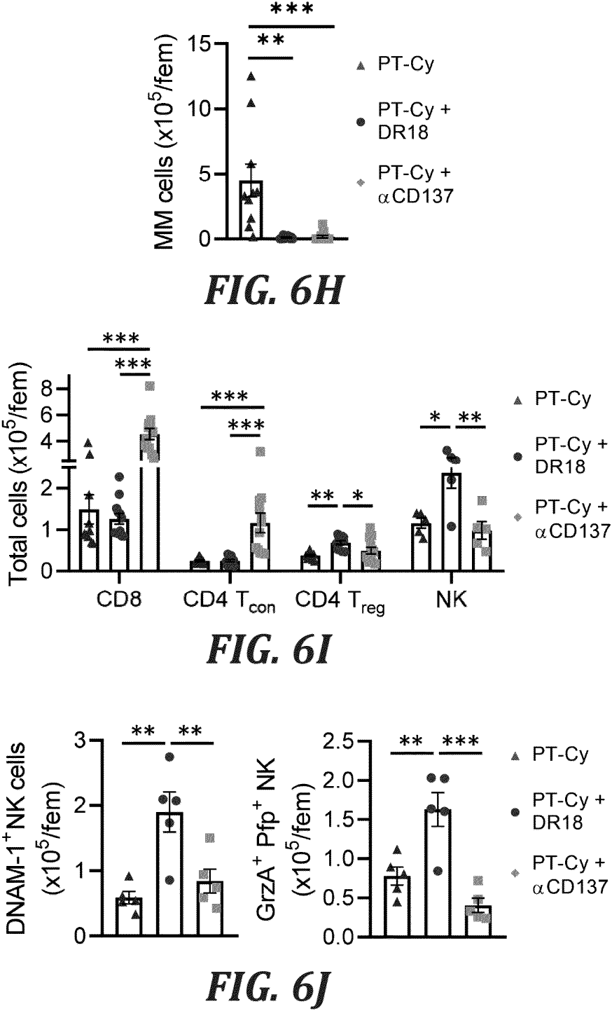

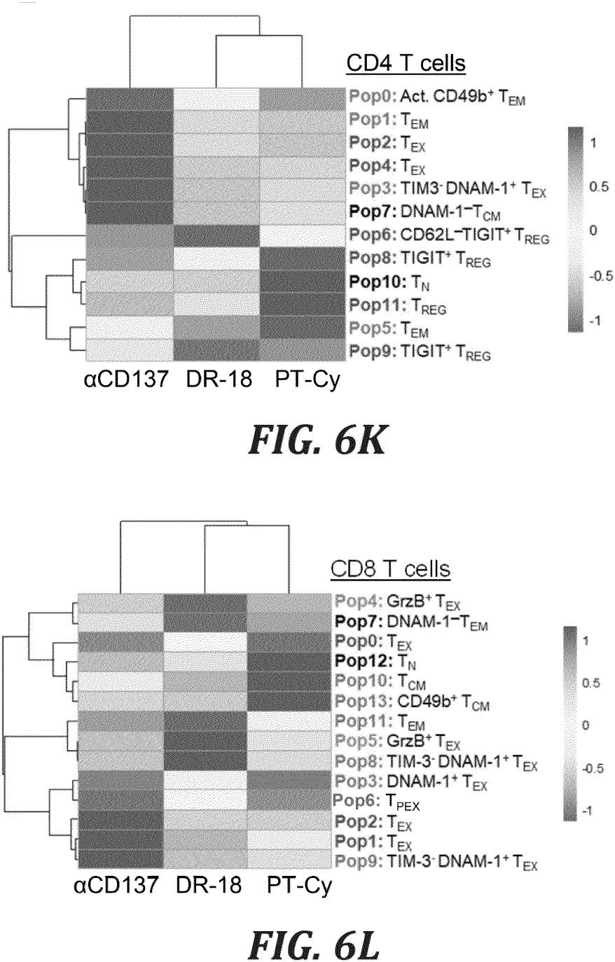

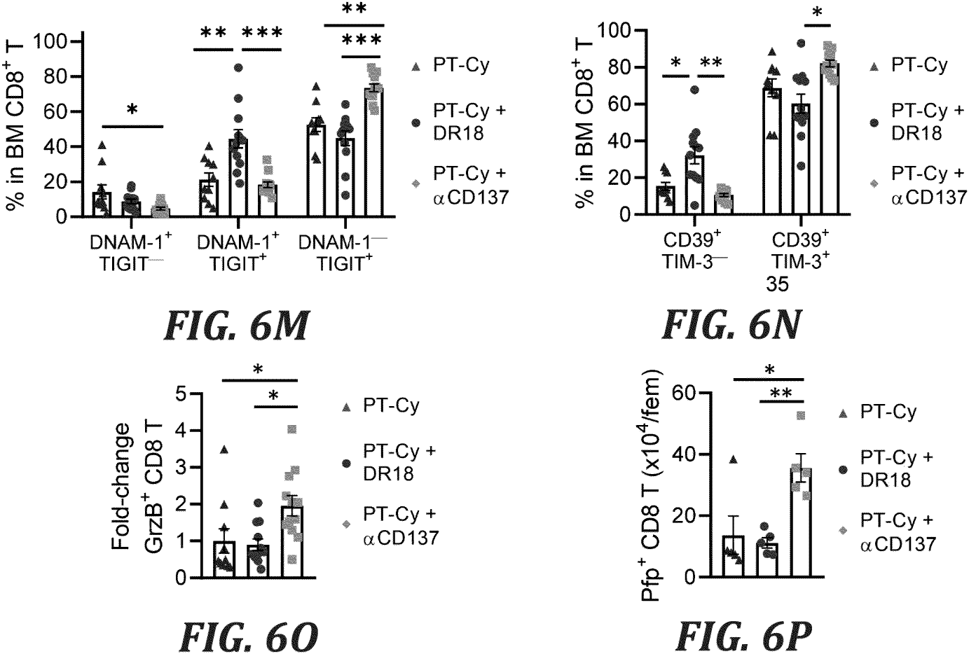

The stem-like memory T cell phenotype can be adequately enriched in the donor immune cell population (e.g., after one or more treatments, e.g., PT-Cy treatment), and the donor immune cells can be stimulated, for example, with one or more agonist immunotherapies, to enhance specific GVT responses without increasing risk of GVHD (FIG. 6D). While any of various immune cell stimulation strategies can be employed without departing from the scope of the disclosure, in example embodiments as disclosed herein, the stimulating comprises administering an agonist immunotherapy to the subject for expansion of activated CD8 T cells (FIG. 6F), expansion of natural killer (NK) cells (FIG. 6G), or both expansion of activated CD8 T cells and expansion of NK cells. The agonist immunotherapy can include administering an anti-CD137 antibody treatment, e.g., a therapeutically effective amount of an anti-CD137 antibody treatment, to the subject, and/or can include administering a decoy-resistant IL-18 (DR-18) treatment e.g., a therapeutically effective DR-18 treatment, to the subject.

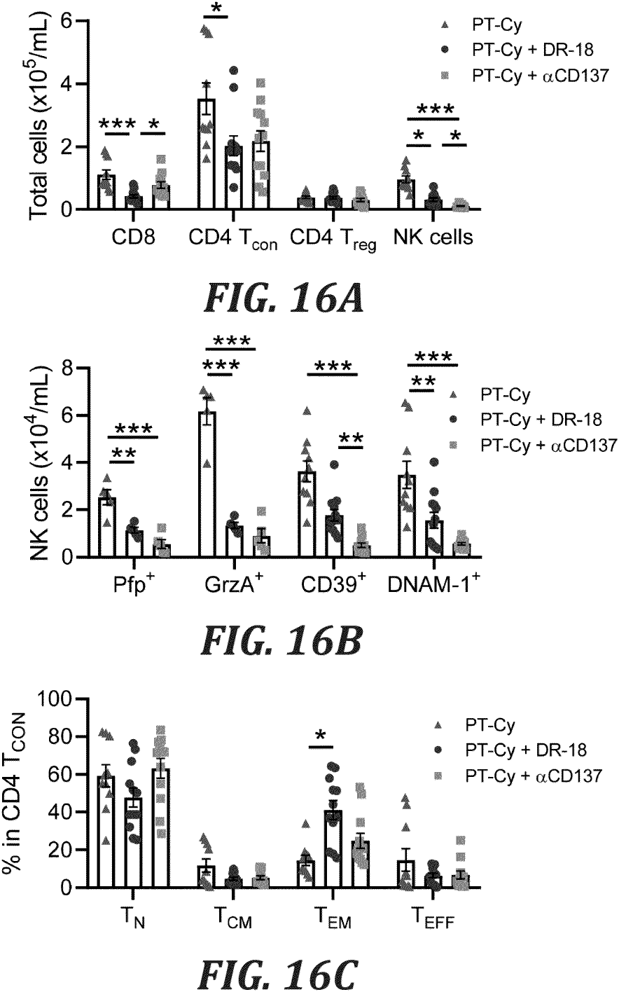

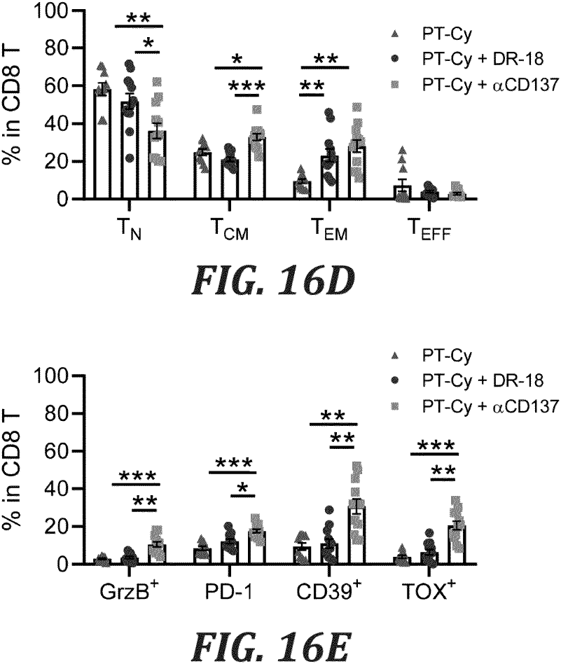

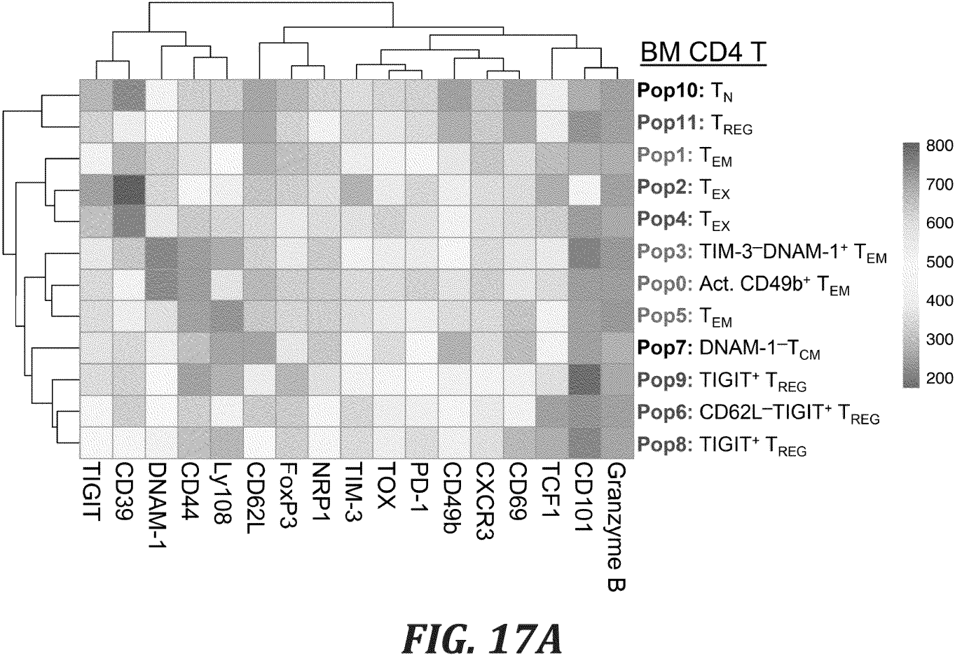

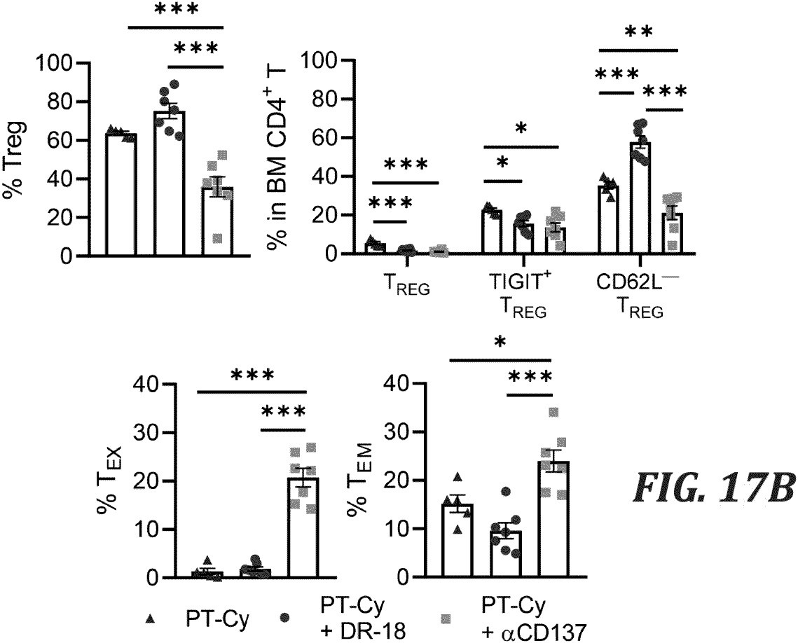

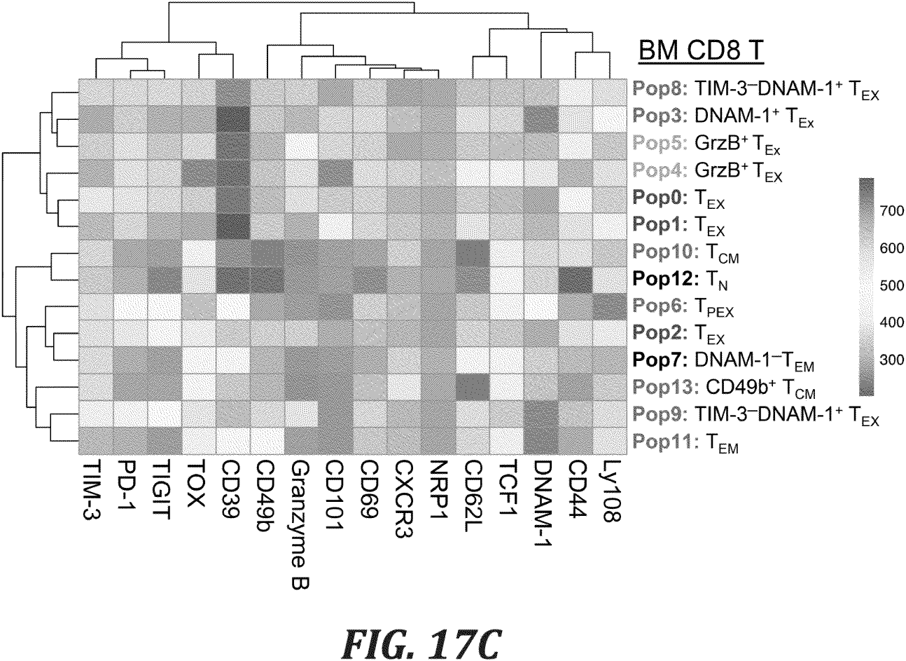

While an anti-CD137 antibody treatment, a DR-18 treatment, both, and/or one or more other agonist immunotherapies can promote immune cell activation and increase anti-tumor immunity, in at least some instances, a beneficial agonist immunotherapy can cause and/or can be associated with any of the following characteristics, in whole or in part or in any combination thereof: (1) a significant increase in the concentration of serum IFN-γ and, to a lesser extent, an increase in the concentration of serum TNF (FIG. 6E); (2) preferential expansion of the activated/effector T cell subset of CD8⁺ T cells (FIG. 6F); (3) expansion of NK cells (FIG. 6G); (4) partial or complete elimination of malignant (e.g., multiple myeloma (MM)) cells from the bone marrow (BM) of the subject (FIG. 6H); (5) an expansion of CD8⁺ and CD4⁺ T cells in BM but not in blood of the subject (FIG. 6I and FIG. 16A); (6) an increase of the number of DNAM-1⁺ and cytolytic NK cells in the marrow but not in the blood (FIG. 6I, FIG. 6J, FIG. 16A, and FIG. 16B); (7) a reduction in regulatory T (Treg) frequency with an increase in exhausted and effector CD4⁺ T cell subsets (FIG. 17B); expansion of a CD62L⁻ Treg population (FIG. 17C); (8) CD8⁺ T cell activation with a relative enrichment in non-exhausted effector populations (FIG. 6L and FIG. 17C) and increased frequency of DNAM- 1+TIGIT⁺ and CD39intTIM3⁻ subsets (FIG. 6M and FIG. 6N); (9) a prevalence of terminally exhausted CD8⁺ T cells and/or an increase of the total number of cytotoxic granzyme B⁺ or perforin⁺ CD8⁺ T cells (FIG. 6O and FIG. 6P); (10) a reduction of the total number of CD4⁺ and CD8⁺ T cells but an increase of the frequency of T_{EM} in both blood and BM (FIG. 16A, FIG. 16C, and FIG. 16D); and (11) increased expression of granzyme B, PD-1, CD39, and TOX on CD8⁺ T cells in the blood but not to the same extent as observed in the BM (FIG. 16E). In at least some instances, a DR-18 treatment can generate a less terminally exhausted phenotype compared with an anti-CD137 treatment, potentially due to the expression of IL-18R on stem-like CD8⁺ and CD4⁺ T cells after the PT-Cy treatment.

The methods can include and/or be combined with one or more other established or experimental treatments. For example, in embodiments, a method can further comprise administering a donor lymphocyte infusion (DLI) to the subject. A DLI is a blood cell infusion in which CD3⁺ lymphocytes from a donor are infused, after a tissue (e.g., bone marrow) transplant, to augment an anti-tumor immune response or ensure that the donor cells remain engrafted. Generally, the donated white blood cells can contain cells of the immune system that recognize and destroy cancer cells.

In yet another aspect, the disclosure provides a method for enhancing a GVT response of a plurality of T cells to treat a hematological malignancy of a subject, the method comprising administering a DR-18 treatment to the subject. In such embodiments, the DR-18 treatment can be a central feature of the method, and other features of the method may be changed or varied in a particular embodiment. In example embodiments, the method further comprises administering a PT-Cy treatment to the subject to enrich for a stem-like memory T cell phenotype in the plurality of T cells; however, alternative strategies for enrichment of the stem-like memory T cell phenotype in the plurality of T cells can be used in a particular embodiment without departing from the scope of the disclosure.

### EXAMPLES

### Example 1: Depletion of Exhausted Alloreactive T Cells Enables Targeting of Stem-Like Memory T Cells to Generate Tumor-Specific Immunity

### Summary

Some hematological malignancies such as multiple myeloma are inherently resistant to immune-mediated antitumor responses, the cause of which remains unknown. Allogeneic bone marrow transplantation (alloBMT) is the only curative immunotherapy for hematological malignancies due to profound graft-versus-tumor (GVT) effects, but relapse remains the major cause of death. Murine models of alloBMT were developed where the hematological malignancy is either sensitive (acute myeloid leukemia (AML)) or resistant (myeloma) to GVT effects. It was found that CD8⁺ T cell exhaustion in bone marrow was primarily alloantigen-driven, with expression of inhibitory ligands present on myeloma but not AML. Because of this tumor-independent exhaustion signature, immune checkpoint inhibition (ICI) in myeloma exacerbated graft-versus-host disease (GVHD) without promoting GVT effects. Administration of post-transplant cyclophosphamide (PT-Cy) depleted donor T cells with an exhausted phenotype and spared T cells displaying a stem-like memory phenotype with chromatin accessibility present in cytokine signaling genes, including the interleukin-18 (IL-18) receptor. Whereas ICI with anti-PD-1 or anti-TIM-3 remained ineffective after PT-Cy, administration of a decoy-resistant IL-18 (DR-18) strongly enhanced GVT effects in both myeloma and leukemia models, without exacerbation of GVHD. Mechanisms of resistance to T cell-mediated antitumor effects after alloBMT and an immunotherapy approach targeting stem-like memory T cells to enhance antitumor immunity are disclosed.

### Introduction

In this example, it was first sought to understand why some hematological malignancies are resistant to GVT effects by developing preclinical models that were sensitive (acute myeloid leukemia (AML)) or resistant (myeloma) to GVT after alloBMT. Second, multiome single-cell sequencing techniques were used to phenotype T cells in the bone marrow (BM) microenvironment and identify pathways that could be targeted to improve GVT effects after alloBMT. Broad, alloantigen-induced CD8⁺ T cell exhaustion was observed that could be reduced with an immunosuppressant used after alloBMT, cyclophosphamide (PT-Cy), which shifted the induction of CD8⁺ T cell exhaustion to a malignancy-driven phenotype at myeloma relapse. Multiome sequencing demonstrated increased chromatin accessibility and RNA expression of the interleukin-18 (IL-18) receptor on stem-like memory CD8⁺ T cells after PT-Cy. Last, several immunotherapies were tested after PT-Cy and it was found that although ICI did not induce lethal GVHD, it also failed to enhance GVT effects. Conversely, an IL-18 cytokine mimetic (DR-18) facilitated potent antitumor responses in both myeloma and leukemia without exacerbating GVHD.

### Results

### Graft-Versus-Myeloma Effects Are Subverted After alloBMT

To determine potential factors underlying the resistance of patients with myeloma to GVT effects, preclinical murine models of transplantation were generated for primary AML and myeloma that were found to be GVT-sensitive and GVT-resistant, respectively. To achieve this, a system of allogeneic BMT was developed where C57B⅙ recipients are transplanted with BM and T cell grafts from minor MHC (major histocompatibility complex)-mismatched C3H.SW donors (alloBMT) or syngeneic C57B⅙ donors (synBMT). A green fluorescent protein (GFP)-expressing MLL-AF9-driven leukemia that allows for monitoring of tumor cells in peripheral blood and Vk*MYC myeloma that secretes immunoglobulin G (IgG), which can be monitored by serum protein electrophoresis as an M-band (albumin/gamma ratio) that is a hallmark of clinical disease, were utilized.

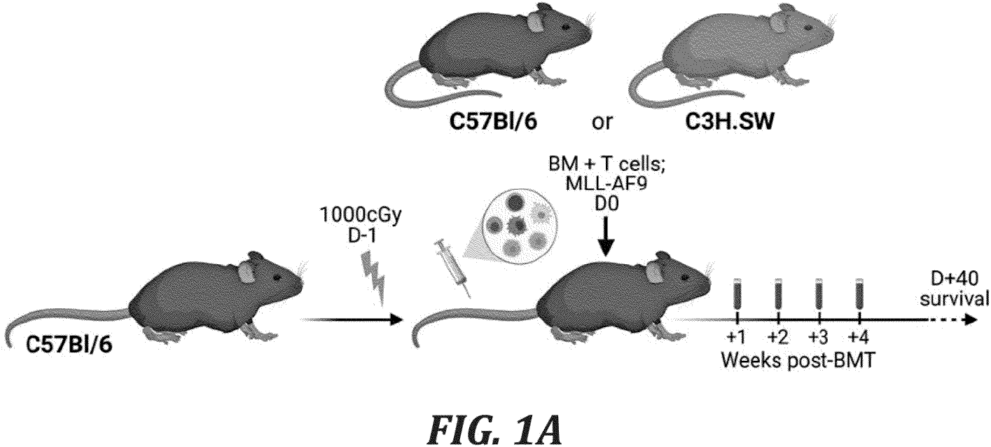

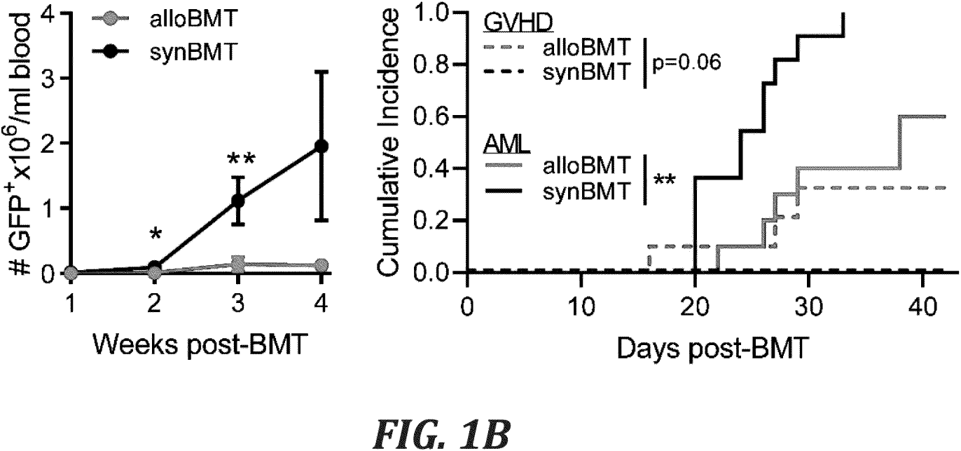

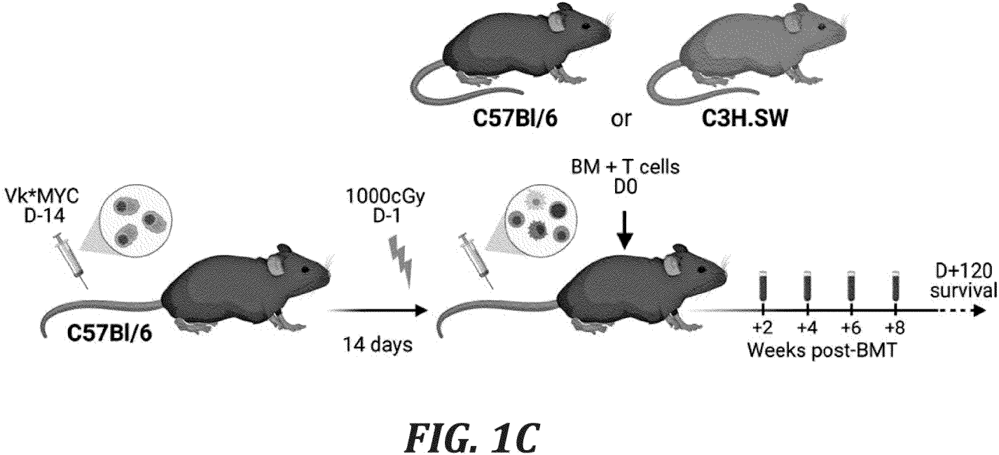

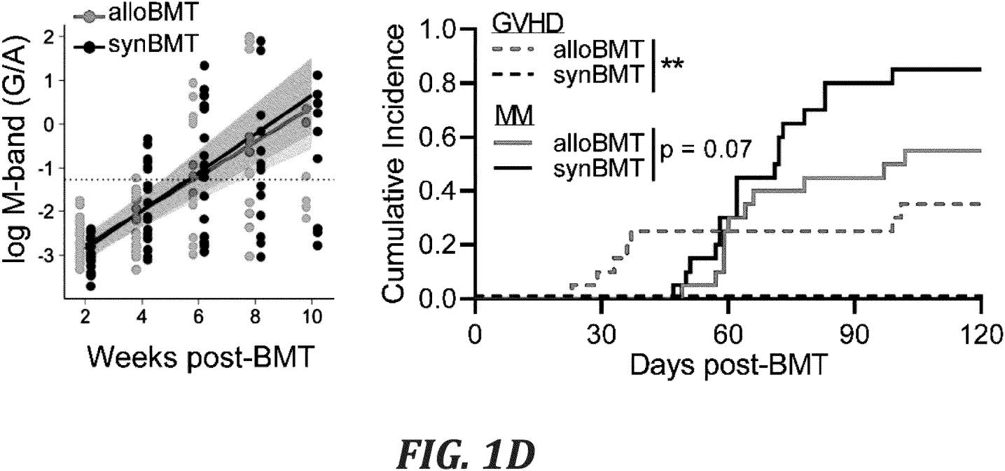

In mice bearing MLL-AF9-driven AML, recipients of allogeneic grafts had significantly reduced circulating leukemia cells and a reduced relapse-related mortality compared with synBMT, confirming an allogeneic graft-versus leukemia (GVL) effect (FIG. 1A and FIG. 1B). Mice with late leukemia related deaths in the alloBMT group succumbed to marrow failure, consistent with systemic immune control but local escape from GVL effects in the BM. In contrast, ineffective graft-versus myeloma (GVM) responses were seen in Vk*MYC myeloma- bearing recipients, with no significant difference in the rate of myeloma growth as determined by M-band progression. Furthermore, competing risk analysis revealed that there was a significantly increased risk of GVHD in alloBMT recipients compared with synBMT that outweighed any potential GVM effects (FIG. 1C and FIG. 1D). Thus, although an effective GVT response could be generated after alloBMT against AML, this was subverted in MM-bearing recipients, an observation that recapitulates clinical data demonstrating that myeloma is largely resistant to GVT effects. Mechanisms underpinning this GVT resistance are of interest and are broadly applicable clinically, because some patients with AML have reduced GVT sensitivity relative to highly sensitive malignancies (e.g., chronic myeloid leukemia).

Donor CD8⁺ T cells undergo exhaustion in response to allogeneic rather than tumor antigens after alloBMT

To determine the mechanisms responsible for the ineffective GVM response after alloBMT, immune phenotyping of CD8⁺ T cells in the BM between 2 and 8 weeks after transplantation was performed using flow cytometry. These experiments were performed in the absence of myeloma (MM-free) to control for the effects of concurrent myeloma on CD8⁺ T cell function after BMT. CD8⁺ T cells were focused on initially because GVHD in this model is primarily MHC class I dependent, and CD8⁺ T cells are crucial to long-term myeloma-specific immunity after autologous BMT.

Multidimensional t-distributed stochastic neighbor embedding (t-SNE) analysis of the flow cytometry data was used and differential clustering of allogeneic and syngeneic CD8⁺ T cells was observed by 2 weeks after transplant (FIG. 2A). This phenotype persisted through 8 weeks after transplant. A significant expansion of CD8⁺ T cells was noted 2 to 4 weeks after transplant in alloBMT recipients, which were predominately effector memory T cells (CD44⁺CD62L⁻, T_{EM}; FIG. 2B and FIG. 2C). Conversely, synBMT recipients had equivalent CD4⁺ and CD8⁺ T cell expansion with higher frequencies of central memory (CD44⁺- CD62L⁺, T_{CM}) versus T_{EM} CD8⁺ T cells (FIG. 2B and FIG. 2C). Most CD8⁺ T cells expressed TIGIT, PD-1, and TIM-3 early after alloBMT, and both PD-1 and TIGIT expression persisted long term (FIG. 2D). DNAM-1 expression was maintained on a proportion of CD8⁺ T cells after alloBMT, suggesting that these T cells were either activated or at an early stage of exhaustion (FIG. 2A). Expansion of these alloreactive CD8⁺ T_{EM} cells after alloBMT would be expected to result in enhanced tumor control relative to synBMT, but this was only seen in response to AML, suggesting that the subversion of GVM may reflect tumor-related differences either intrinsic to myeloma or related to differential effects exerted by myeloma (versus AML) on the tumor microenvironment (TME). Therefore, the expression of relevant inhibitory receptor ligands on the cell surface of Vk12653 myeloma and MLL-AF9 AML was investigated. Differential expression of both CD155 and PDL1, the ligands for TIGIT and PD-1, respectively, were noted on Vk*MYC compared with MLL-AF9 (FIG. 2E). This expression of CD155 on malignant cells has been demonstrated to infer resistance to T cell-dependent antitumor immunity. Furthermore, TIGIT has a much higher affinity than DNAM-1 for CD155 and will outcompete for ligand binding even if DNAM-1 expression is maintained on CD8⁺ T cells. Therefore, donor CD8⁺ T cells expressing high levels of TIGIT⁺ and PD-1⁺ in response to alloantigen were putatively inactivated via interaction with cognate inhibitory receptor ligands expressed by myeloma.

TIGIT inhibition does not enhance GVM after alloBMT

Because CD155 and PD-L1 expression on myeloma cells is a potential mechanism of immune escape after alloBMT, it was explored whether these pathways could be targeted therapeutically to generate GVM effects. PD-1 or TIGIT blockade after synBMT significantly improved myeloma specific immunity. PD-1 inhibition after alloBMT can exacerbate GVHD in both preclinical models and clinical practice. To examine whether TIGIT inhibition would affect GVHD and/or GVM, MM-bearing recipients were treated with TIGIT blocking antibodies. Recipients treated with an Fc-enabled (i.e., live) 4B1-G2a clone, αTIGIT-G2a, after transplant had significantly enhanced mortality compared with isotype control (cIg)-treated mice (FIG. 2F). This was associated with an increase in GVHD clinical scores and GVHD-induced mortality, without an associated improvement in the GVM effect (FIG. 2G, FIG. 2H, FIG. 2I).

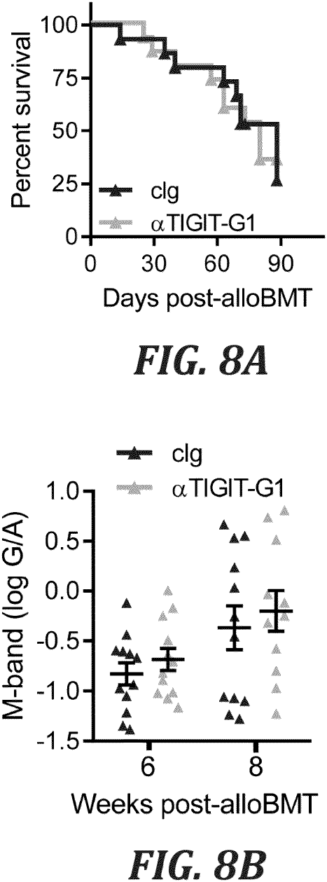

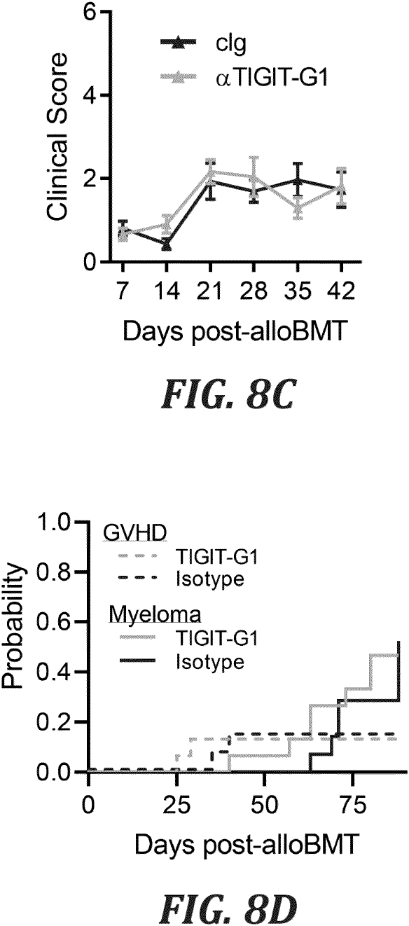

The Fc-dead anti-TIGIT G1-D265A clone (αTIGIT-G1) that does not deplete TIGIT-expressing regulatory T cells was then tested and it was hypothesized that exacerbation of GVHD would be less severe than the Fc-enabled TIGIT. Mice treated with αTIGIT-G1 from 3 weeks after alloBMT had similar overall survival compared to isotype-treated mice (FIG. 8A). GVHD, myeloma burden, and myeloma progression were similar between αTIGIT-G1- and cIg-treated mice (FIG. 8B, FIG. 8C, FIG. 8D). Therefore, TIGIT inhibition was not sufficient to generate GVM responses after alloBMT. This is in contrast to data demonstrating that blockade of myeloma-induced TIGIT expression on CD8⁺ T cells could induce potent myeloma immunity in a synBMT model (where alloantigen is absent). This led us to investigate whether the expression of inhibitory receptors on CD8⁺ T cells was generated by CD8⁺ T cell recognition of malignancy-derived antigens or broadly by recipient alloantigens after alloBMT.

Myeloma itself does not drive T cell exhaustion after alloBMT

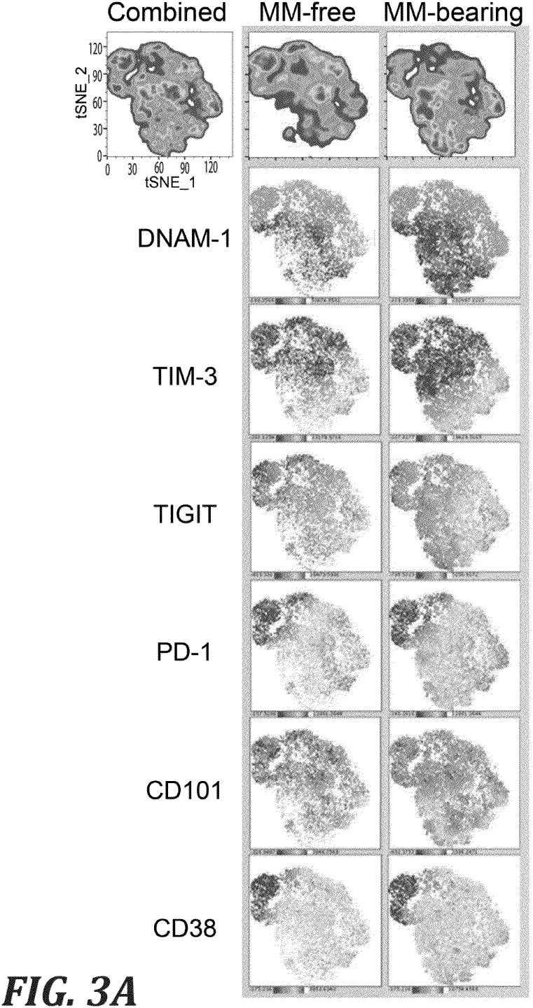

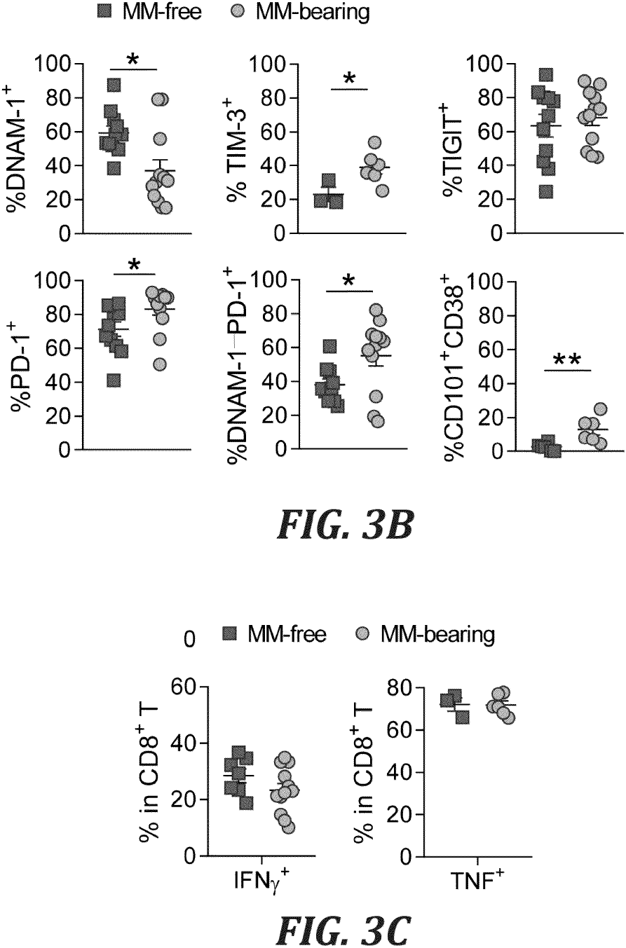

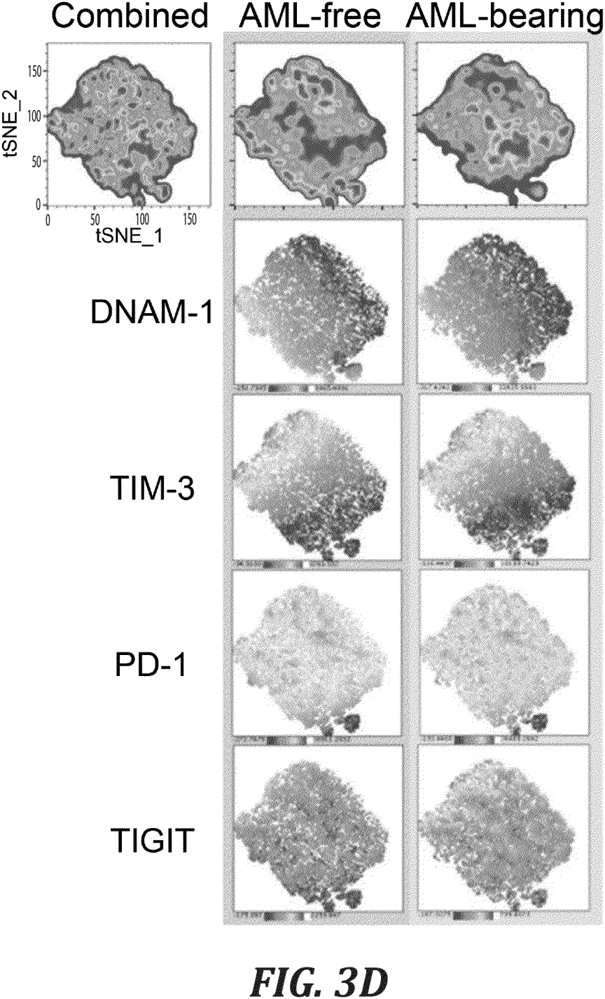

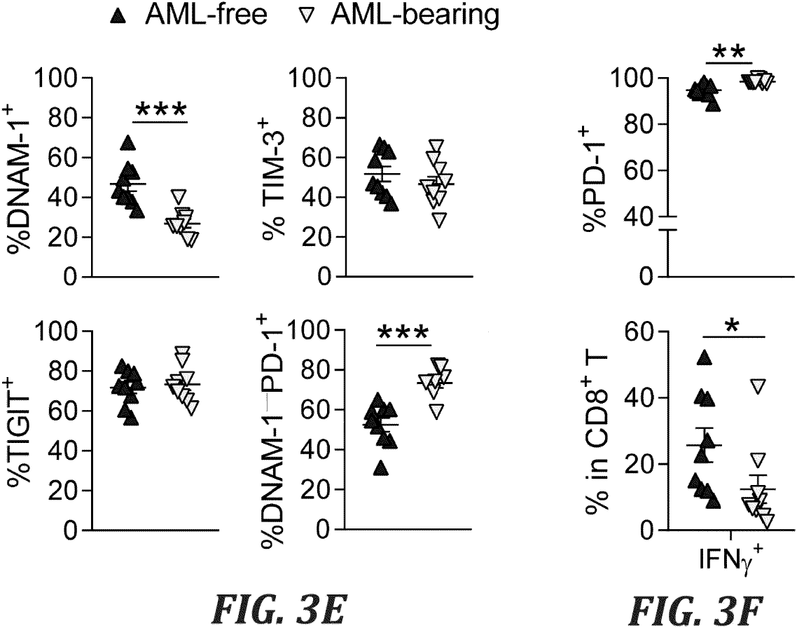

To determine the relative contribution of tumor versus allogeneic antigens to CD8⁺ T cell exhaustion, CD8⁺ T cells in the BM of myeloma-bearing recipients (MM-bearing) or control mice that were transplanted in the absence of myeloma (MMfree) were analyzed at 8 weeks after alloBMT, a time point of active myeloma progression in the MM-bearing cohort. It was noted that only a small increase in PD-1 and TIM-3 expression, and reduced DNAM-1 expression, occurred in MM-bearing versus MM-free controls (FIG. 3A and FIG. 3B). In particular, an increase in the frequency of CD101⁺CD38⁺CD8⁺ T cells was seen in MM-bearing compared with MM-free mice (FIG. 3B), a phenotype that is usually associated with dysfunctional, terminally exhausted T cells. Nonetheless, CD8⁺ T cells from MM-bearing mice did not have alterations in interferon-γ (IFN-γ) or tumor necrosis factor (TNF) production upon ex vivo restimulation (with phorbol 12-myristate 13-acetate (PMA)/ionomycin) after alloBMT compared with MM-free mice (FIG. 3C), likely due to the relatively low frequency of CD38⁺CD101⁺ cells within the CD8⁺ T cell compartment. Together, these data demonstrate that cytokine production by BM CD8⁺ T cells was not adversely affected by the presence of myeloma after alloBMT. Similar analyses in AML-bearing mice also demonstrated a significant increase in PD-1 expression and a concurrent decrease in DNAM-1 expression compared with AML-free mice (FIG. 3D and FIG. 3E). However, in AML-bearing mice the overall frequency of IFN-γ⁺ CD8⁺ T cells was reduced (FIG. 3F), indicating that tumor-induced CD8⁺ T cell dysfunction occurred in this leukemia model. The absence of exaggerated CD8⁺ T cell exhaustion in mice with relapsed myeloma after alloBMT confirms that the main driver of T cell exhaustion in this setting is alloantigen rather than tumor-specific antigen, potentially explaining why TIGIT blockade exacerbated GVHD without promoting GVM (FIG. 2F, FIG. 2G, FIG. 2H, FIG. 2I).

Post-transplant cyclophosphamide attenuates alloantigen-induced CD8⁺ and CD4⁺ T cell exhaustion in the BM after alloBMT

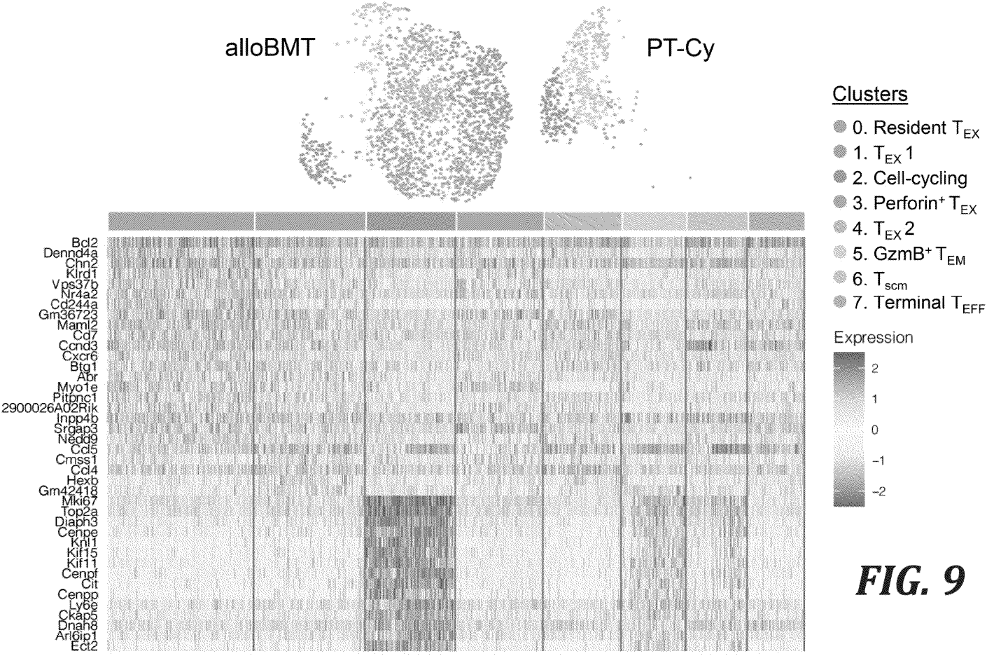

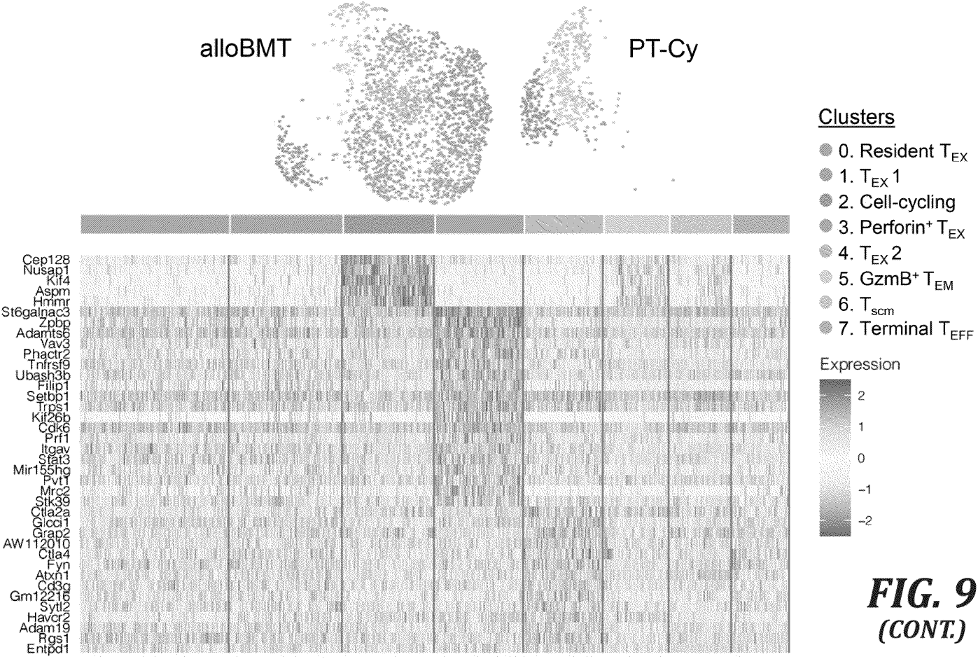

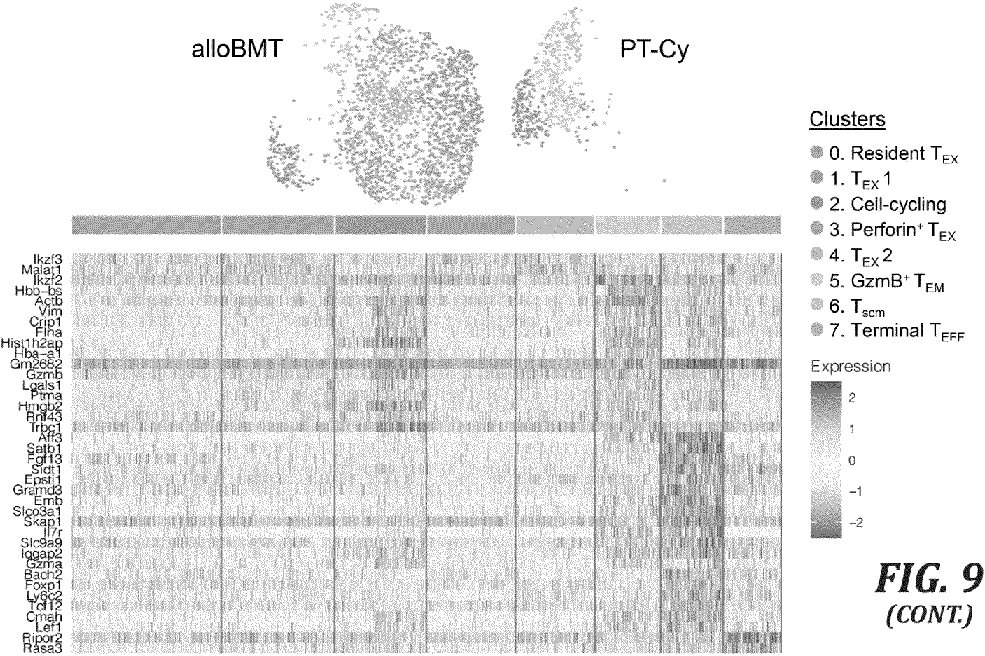

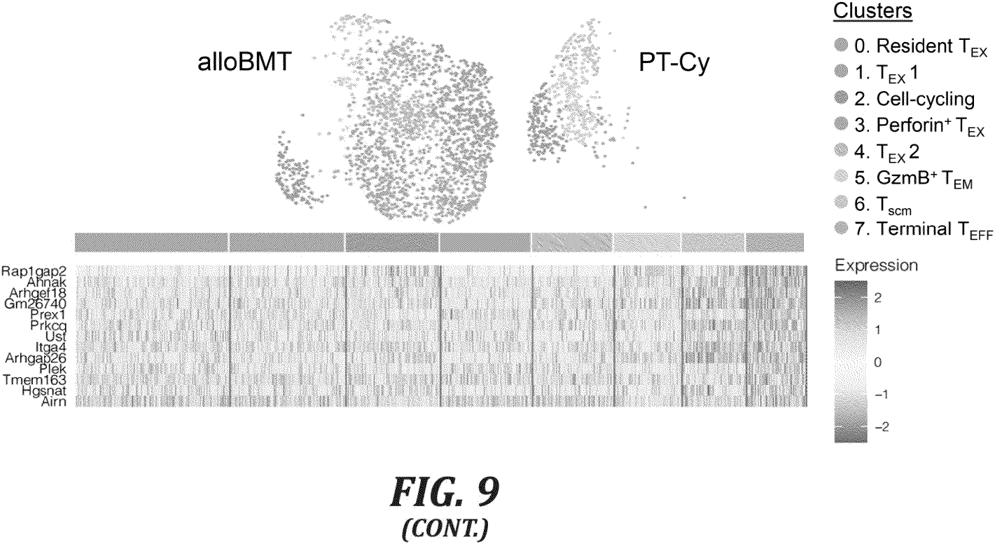

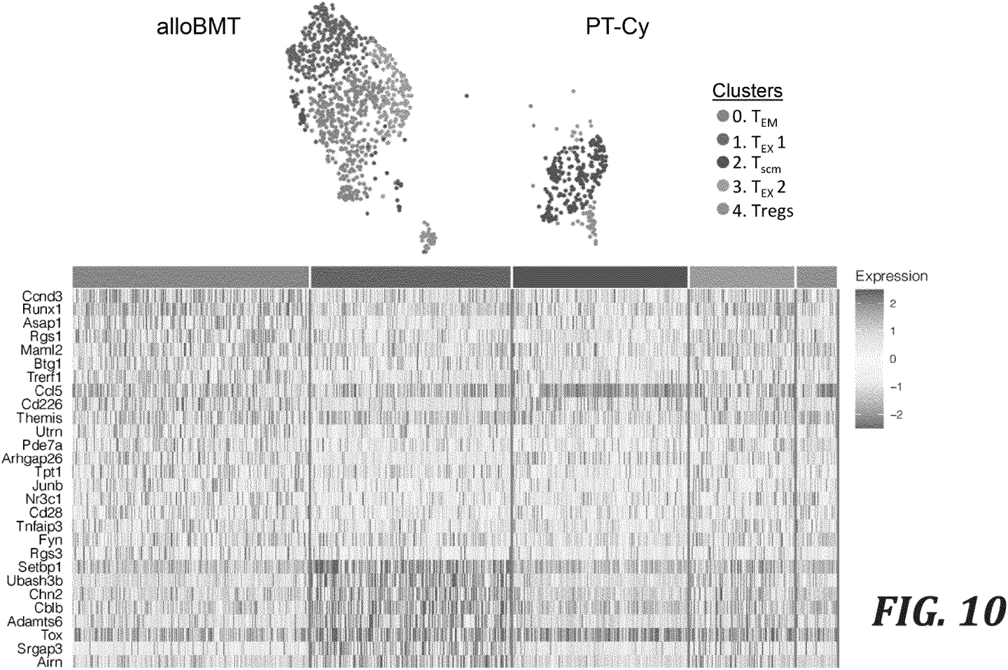

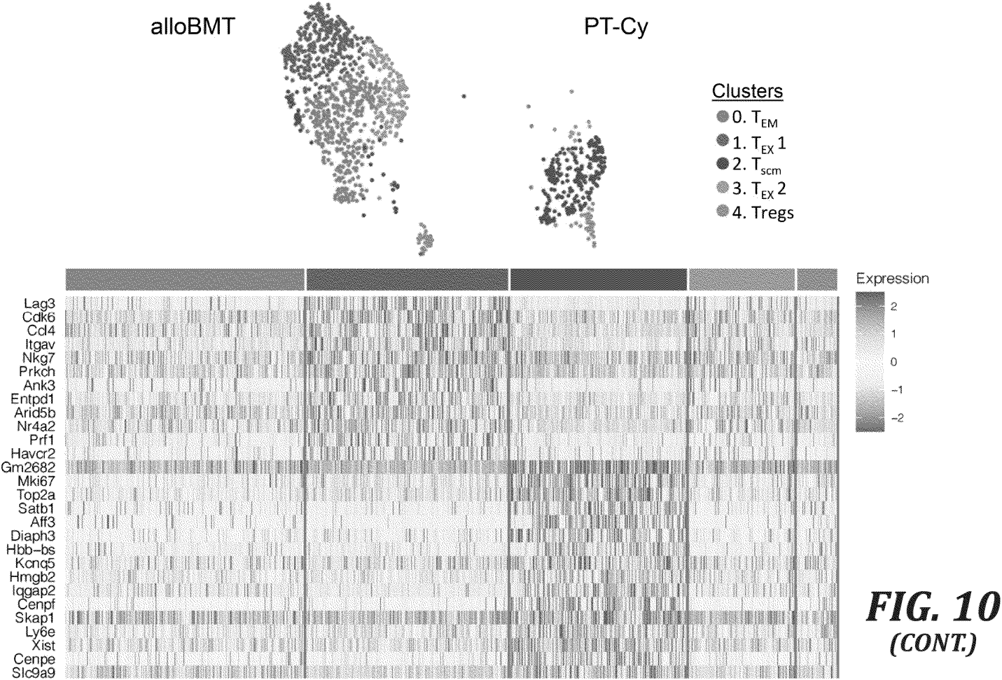

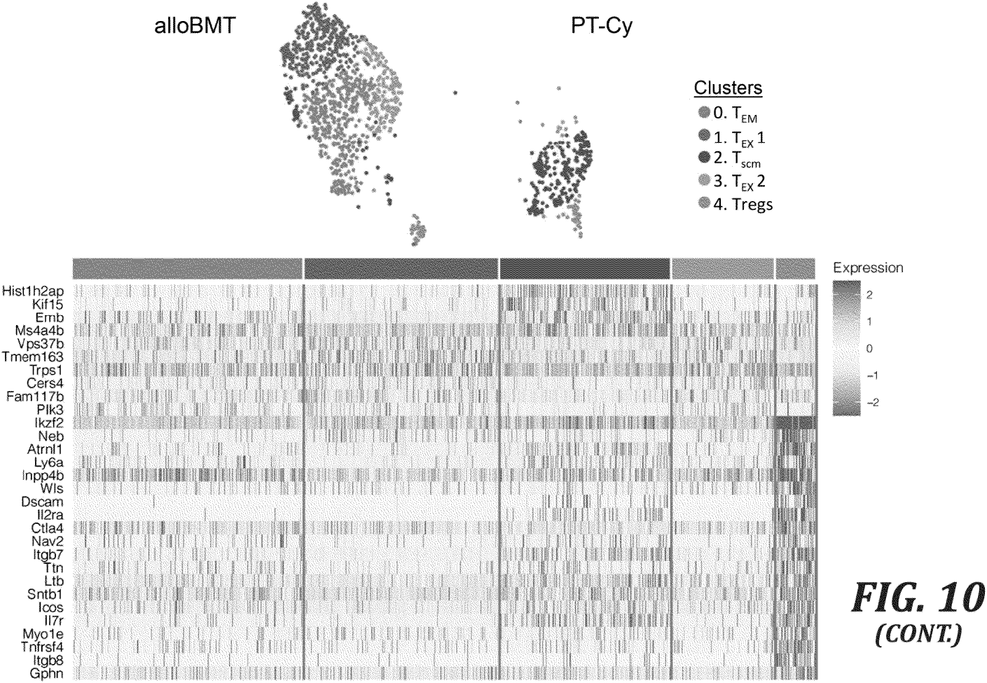

It was next investigated whether it was possible to eliminate highly activated, alloreactive T cells to preserve T cell subsets that could be safely harnessed to improve GVT responses in the BM after alloBMT. To achieve this, alloBMT recipients were treated with a currently used GVHD prophylaxis strategy, post-transplant cyclophosphamide (PT-Cy), that strongly attenuates alloreactive T cell responses and GVHD. T cell phenotypes in the BM were first assessed 14 days after transplantation in MM-free mice to generate a dataset that could be broadly interpreted independent of specific tumor induced phenotypes. Single-cell sequencing was performed on sorted T cells from BM with the 10x Genomics Multiome platform to measure concurrent changes in gene expression (RNA sequencing) and chromatin accessibility (ATAC (assay for transposase-accessible chromatin) sequencing). Unsupervised clustering on the basis of weighted nearest neighbor algorithms identified eight clusters within CD8⁺ (FIG. 9) and five clusters within CD4⁺ (FIG. 10) T cells. Clusters were annotated based on all differentially expressed genes in each cluster (data files S1 and S2), and key genes associated with each cluster in CD8⁺ (FIG. 4A) and CD4⁺ (FIG. 4B) T cells have been highlighted.

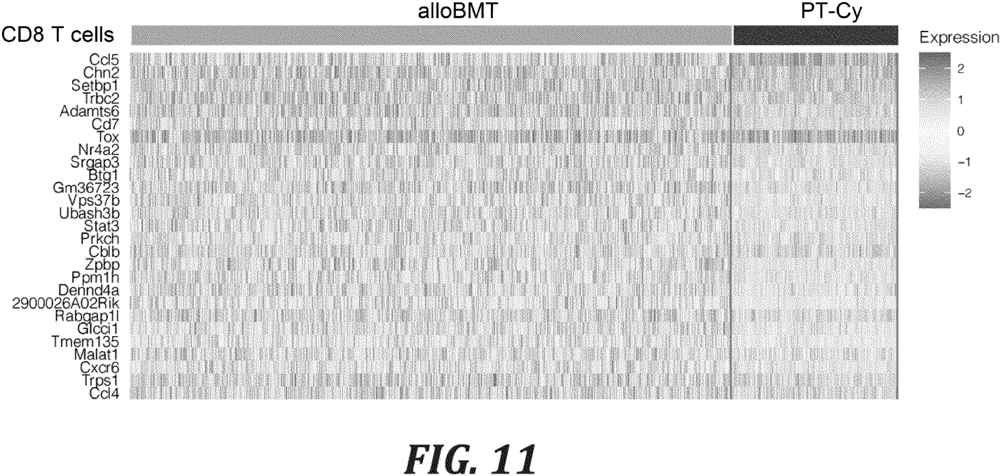

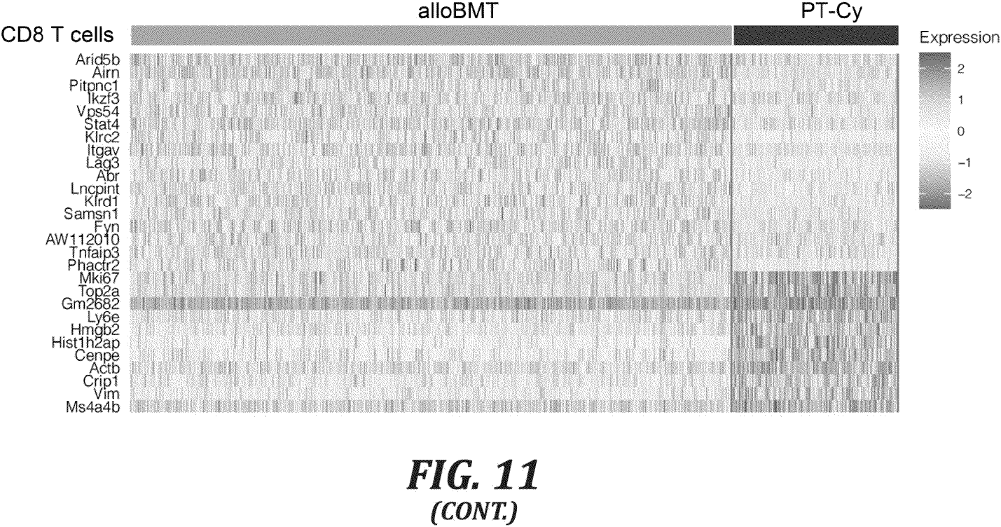

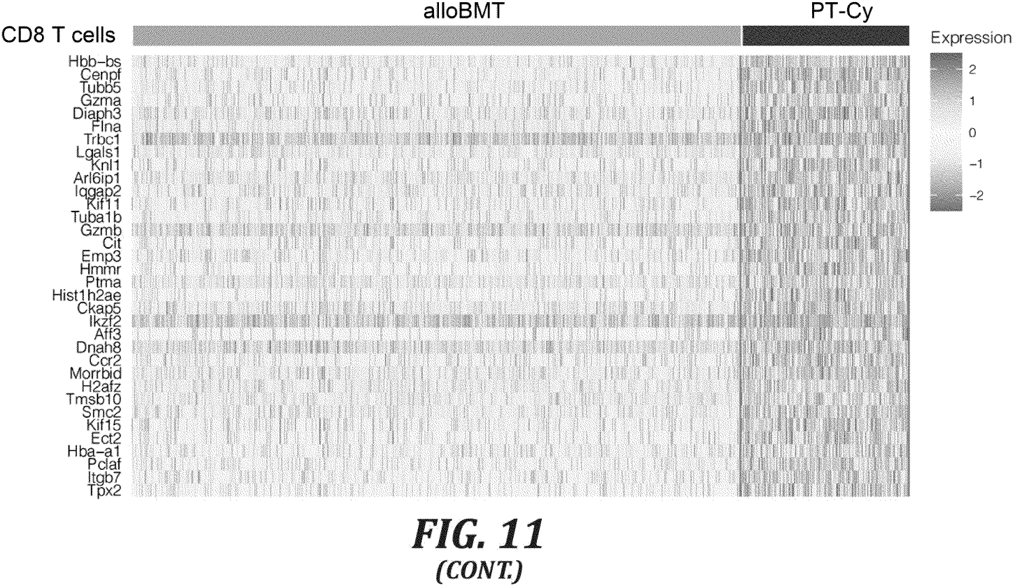

In untreated alloBMT recipients, CD8⁺ T cells highly expressed genes associated with T cell exhaustion, whereas those in PT-Cy- treated alloBMT recipients had higher expression of stem-like memory gene signatures (FIG. 4C and FIG. 11). When T cells were unbiasedly clustered, most CD8⁺ T cells from PT-Cy- treated recipients were within a cluster (number 6) that included stem-like memory cells (T_{SCM}) characterized by Bach2, Ly6a (encoding sca-1), I17r, I118r1, Cd226, and absence of Pdcd1 (FIG. 4A). These changes in gene expression were mirrored by changes in chromatin accessibility: a similar skewing toward stem-like signatures measured by gene accessibility scores (FIG. 4C) was observed. Together, these findings confirm a fundamental change in the phenotype of CD8⁺ T cells surviving PT-Cy. Analogous to the CD8⁺ T cell compartment, CD4⁺ T cells from alloBMT recipients were enriched for the same exhaustion signature, whereas those in PT-Cy-treated recipients were largely enriched for a T_{SCM} signature (FIG. 4D and FIG. 12). Furthermore, chromVAR analysis highlighted motifs associated with exhaustion (i.e., NR4A1 and NFATC) in T cells from control alloBMT recipients, whereas T cells from PT-Cy-treated alloBMT recipients had motifs associated with stemness (i.e., TCF7 and KLF) (FIG. 13A and FIG. 13B). To identify functional relevance of PT-Cy-driven epigenetic changes, chromatin accessibility in key cytokine receptor genes was observed and it was noted that Il18r1 (IL-18R), I12ra (IL-2Rα), and Il7r (IL-7R) demonstrated increased gene activity scores after PT-Cy in both CD8⁺ and CD4⁺ T cells (FIG. 4E and FIG. 4F).

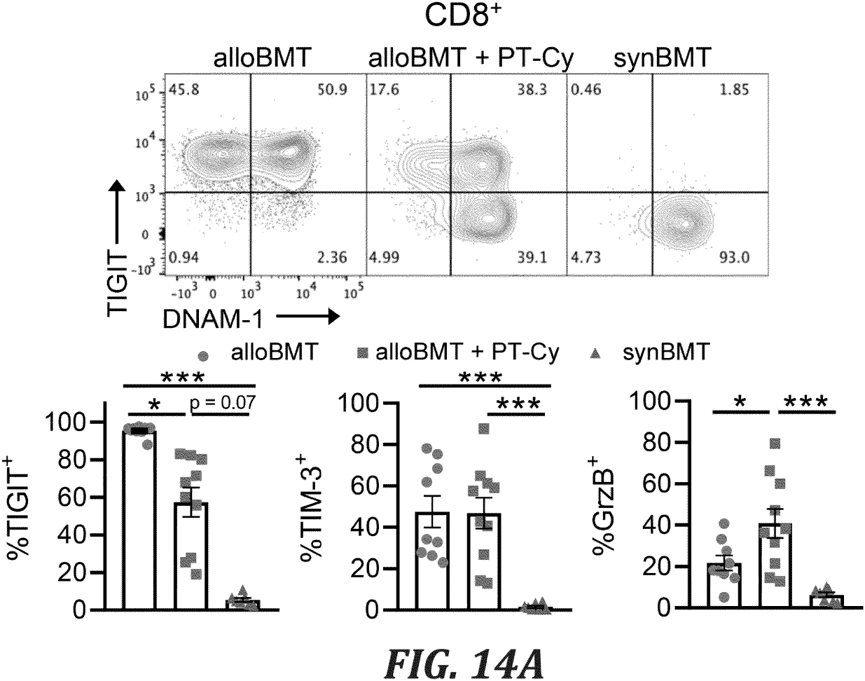

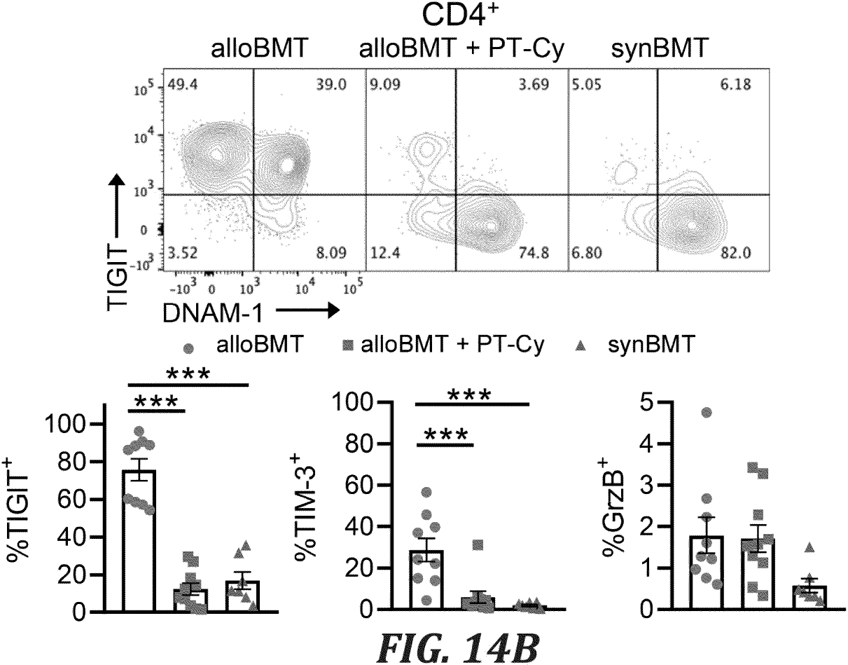

Last, the sequencing data of BM T cells from alloBMT recipients with and without PT-Cy was corroborated with flow cytometry at the same time point, including synBMT recipients as a baseline for any immune effects of transplantation itself. It was confirmed that CD8⁺ T cells had an exhausted phenotype after alloBMT, characterized by expression of high levels of TIGIT, PD-1, TOX, and TIM-3 proteins, whereas syngeneic T cells were DNAM-1⁺ without inhibitory ligand expression (FIG. 4G and FIG. 4H, and FIG. 14A). CD8⁺ T cells from PT-Cy-treated alloBMT recipients had significantly increased DNAM-1 and reduced TIGIT, PD-1, and TOX expression, with a phenotype intermediate to T cells from alloBMT recipients without PT-Cy and recipients of synBMT (where alloantigen was absent). PT-Cy treatment did not abrogate granzyme B production by CD8⁺ T cells, as determined by both RNA and protein expression (FIG. 11 and FIG. 14A). The marked effect of PT-Cy on the CD4⁺ T cell compartment was also confirmed by flow cytometry such that the CD4⁺ T cell exhaustion signature in FoxP3⁻ conventional CD4⁺ T cells from PT-Cy-treated recipients was largely indistinguishable from synBMT recipients, consistent with a dominant effect on class II-dependent alloreactivity (FIG. 4I and FIG. 4J, and FIG. 14B). Together, these data demonstrate that PT-Cy reduced the frequency of alloreactive, exhausted T cells in the BM of alloBMT recipients and instead enriched for T_{SCM} populations, offering a platform on which to subsequently generate tumor-specific responses.

PT-Cy is permissive of myeloma-driven T cell exhaustion after alloBMT

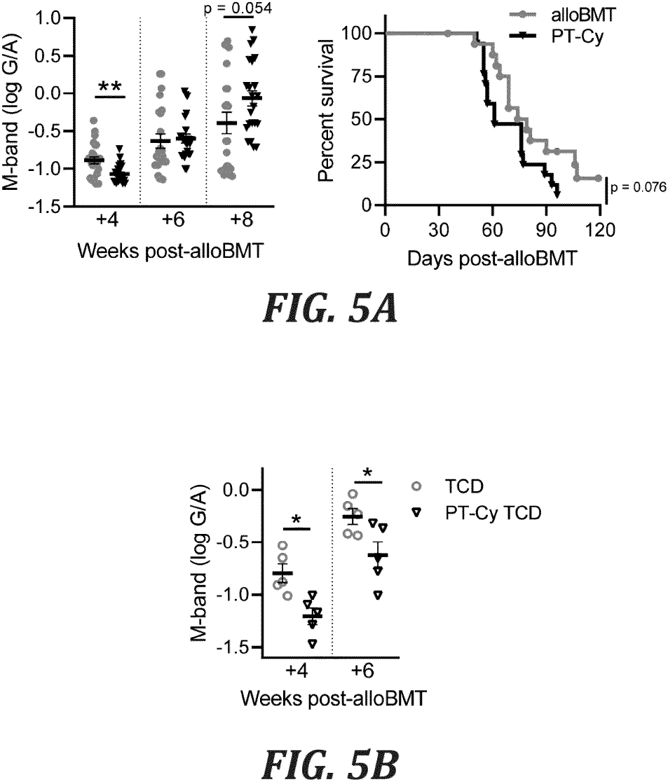

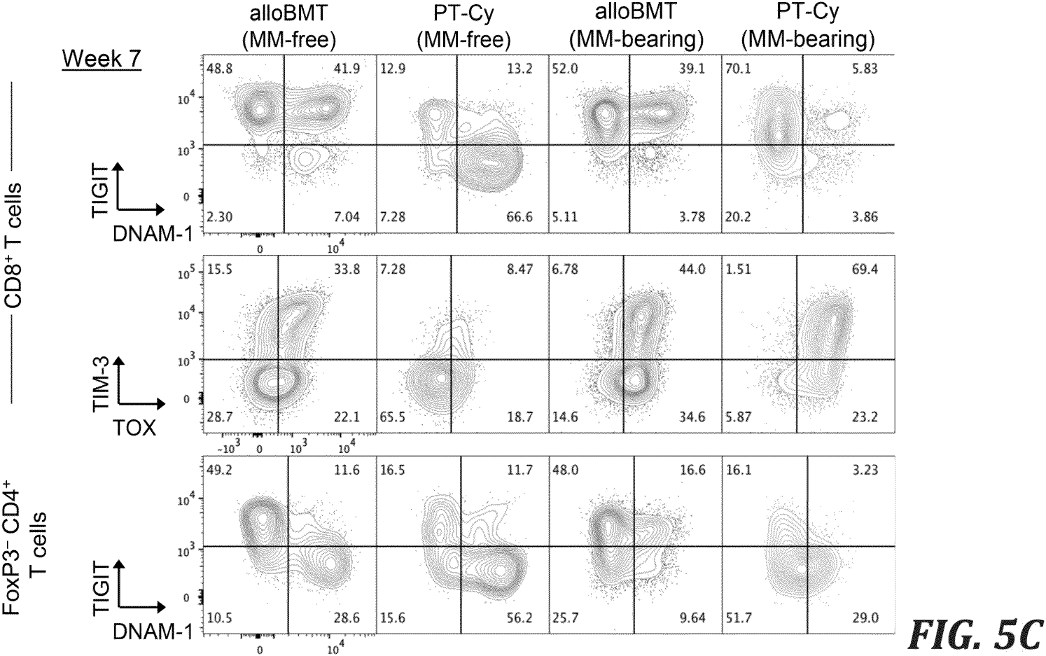

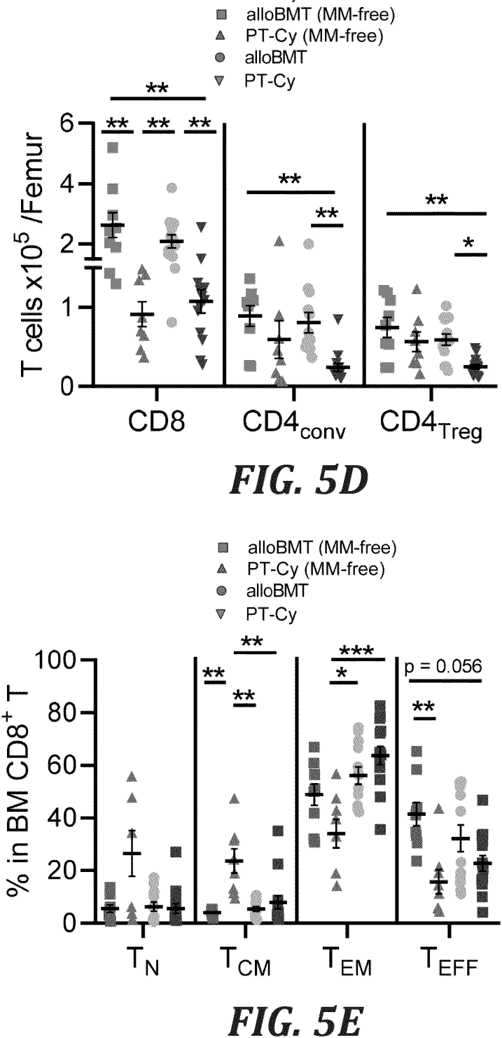

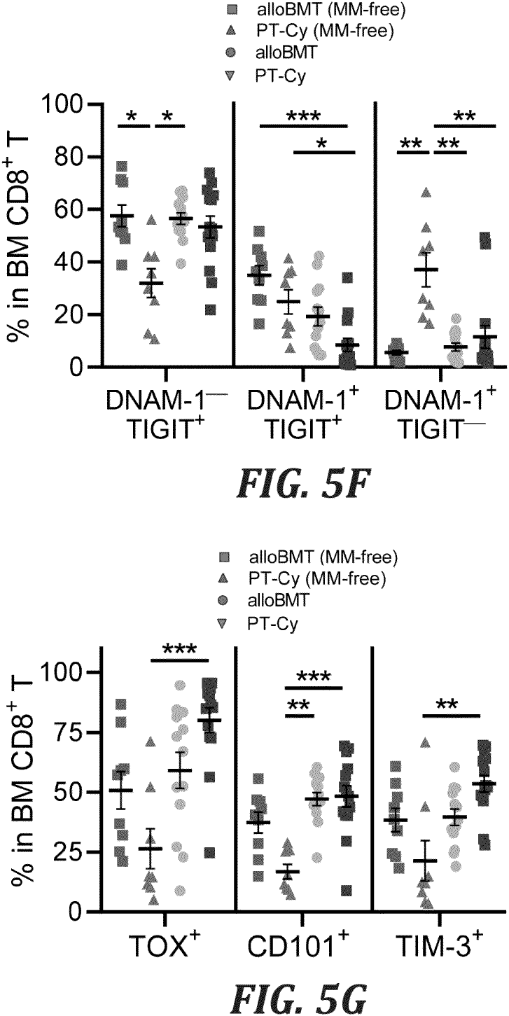

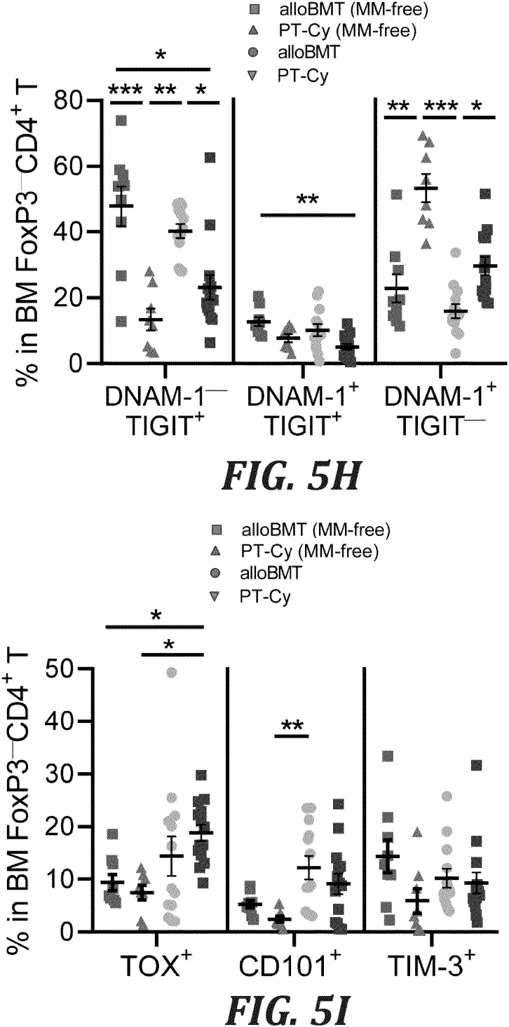

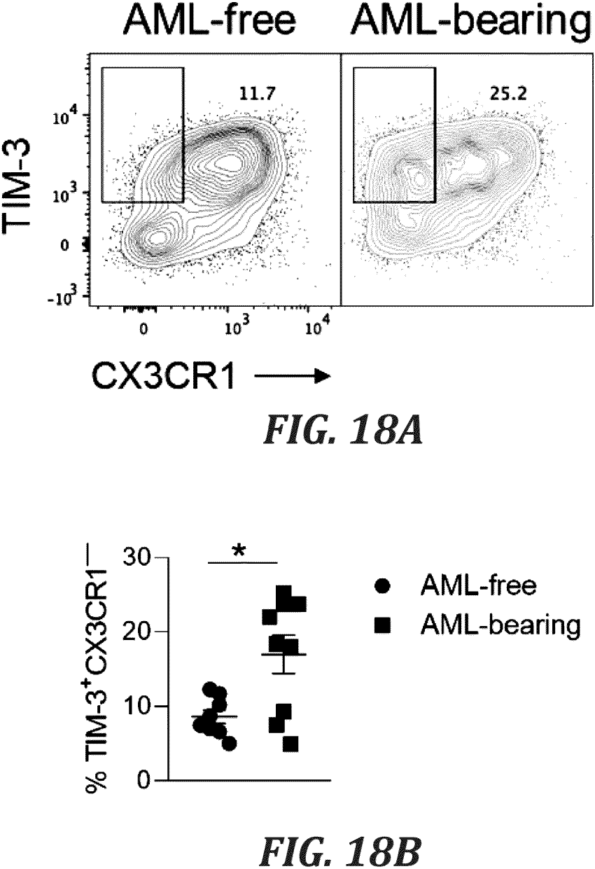

MM-bearing recipients were then treated with PT-Cy after alloBMT and myeloma growth and survival was compared with untreated recipients to determine whether the loss of putative alloreactive T cells in the BM affected the control of myeloma. PT-Cy resulted in early myeloma cytoreduction with reduced M-bands at 4 weeks after alloBMT that was also seen in recipients of T cell-depleted BM (FIG. 5A and FIG. 5B), consistent with the expected cytoreduction mediated by cyclophosphamide. However, this PT-Cy effect was not durable, because there was a subsequent increase in myeloma progression as determined by M-band 8 weeks after alloBMT in PTCy- treated recipients (FIG. 5A). Consistent with this effect, total T cell numbers were also concurrently reduced by PT-Cy at D+14 and D+21 after alloBMT (FIG. 18A and FIG. 18B). Together, these data suggest that direct myeloma cytoreduction partially counteracts the loss of alloreactive T cells mediating GVT effects after PT-Cy.

Having established that PT-Cy reduced alloantigen-induced T cell exhaustion, enriched for a T_{SCM} phenotype, and did not significantly reduce overall survival, it was next sought to determine whether the presence of myeloma in the BM would alter T cell phenotypes in PT-Cy-treated alloBMT recipients with relapsed disease. CD8⁺ and CD4⁺ T cells were analyzed at 7 weeks after transplant from the BM of MM-bearing and MM-free allograft recipients that were treated with and without PT-Cy. In untreated recipients, there was high expression of TIGIT, TIM-3, and TOX on CD8⁺ T cells regardless of whether the mice were MM-free or had active myeloma, indicative of broad alloantigen-driven T cell exhaustion (FIG. 5C, FIG. 5F, and FIG. 5G). In MM-free PT-Cy-treated recipients, most CD8⁺ T cells were DNAM-1⁺TIGIT⁻ and did not express TOX or TIM-3 (FIG. 5C, FIG. 5F, and FIG. 5G), consistent with maintained depletion of exhausted alloreactive T cells observed at D+14 (FIG. 4). However, in MM-bearing PT-Cy-treated recipients, most CD8⁺ T cells were DNAM-1-TIGIT⁺ with high expression of TOX and TIM-3 (FIG. 5C, FIG. 5F, and FIG. 5G), consistent with the onset of myeloma-driven T cell exhaustion. Even at this late time point, the reduction in total numbers of CD4⁺ and CD8⁺ T cells in PT-Cy-treated recipients was maintained (FIG. 5D). In PT-Cy-treated MM-free recipients, there was an increase in CD8⁺ central memory (T_{CM}; CD44⁺CD62L⁺) and a decrease in terminal effector (T_{EFF}; CD44⁻CD62L⁻) T cells, consistent with maintenance of the memory populations that were identified at earlier time points (FIG. 4C and FIG. 5E). Increased expression of TOX and inhibitory receptors, including the terminal exhaustion markers TIM-3 and CD101, on effector cells in MM-bearing compared with MM-free PT-Cy-treated recipients is consistent with the expansion of exhausted, putatively myeloma-specific T cells, a phenotype observed at MM progression after synBMT. Therefore, PT-Cy effectively eliminated alloantigen-driven CD8⁺ T cell exhaustion and enabled exhaustion to instead be driven by myeloma. Although there was a marked effect on CD4⁺ T cells after PT-Cy, there were only subtle differences in this compartment in MM-free versus MM-bearing recipients, including an increase in TOX but not TIGIT or TIM-3 expression (FIG. 5C, FIG. 5H, and FIG. 5I). This finding is unsurprising given the high expression of MHC class I and absence of MHC class II on Vk*MYC myeloma cells. Last, the presence of tumor-driven T cell exhaustion after PT-Cy was confirmed in the MLL-AF9 AML model. An increased frequency of TIM-3⁺CX3CR1⁻ CD8⁺ T cells was observed at 3 weeks after transplant in mice with relapsed AML compared with AMLfree recipients after PT-Cy (FIG. 18A, FIG. 18B). Expression of CX3CR1 was used to exclude transitionary effector cells that contaminate the TIM-3⁺ population at earlier time points in tumor progression.

Agonist immunotherapies beneficially promote GVM after PT-Cy

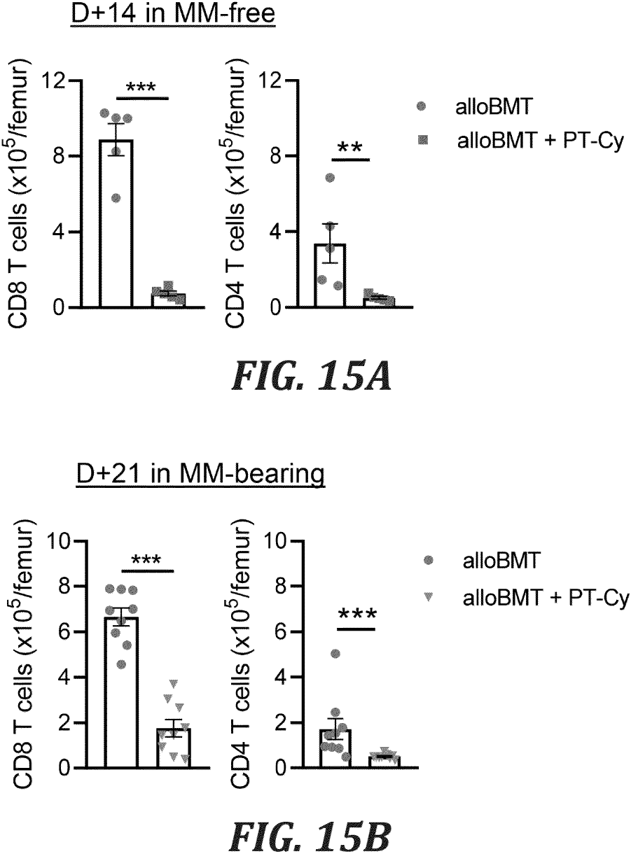

Given the presence of myeloma-driven expression of inhibitory receptors on CD8⁺ T cells at relapse after PT-Cy, the antimyeloma efficacy of ICI in PT-Cy-treated alloBMT recipients was tested (FIG. 6A). ICIs, either anti-PD-1 or anti-TIM-3, were administered from D+7 for 4 weeks and no reduction in myeloma burden was observed in ICI-treated mice (FIG. 6B and FIG. 6C). For ICI to be effective, there may be an appropriate ratio of ICI-responsive T cells to tumor burden. PT-Cy resulted in reduced expression of inhibitory receptors (FIG. 4C) in the context of strongly reduced overall T cell numbers (FIG. 15A and FIG. 15B) at the time point where ICI was administered.

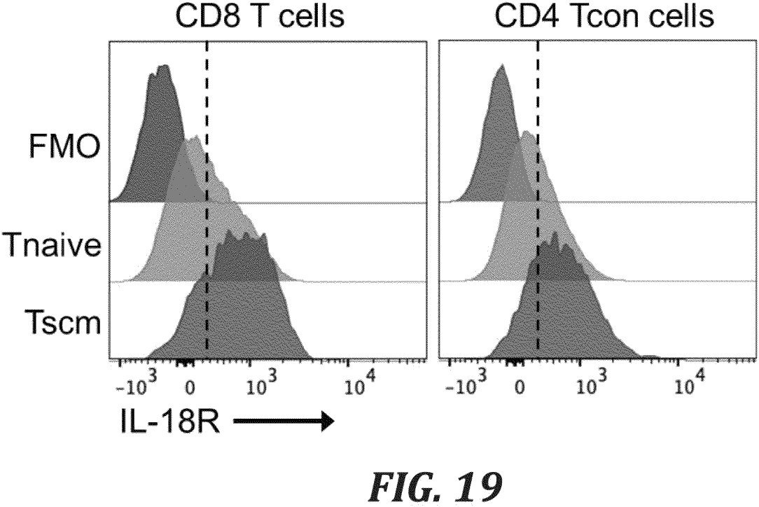

These data suggest that immunotherapies targeting a “brake” on T cell function may be unlikely to drive effective anti-myeloma responses, at least in this setting. To that end, two immunotherapies with known direct agonist activity were investigated, decoy-resistant IL-18 (DR-18) and anti-CD137 (4-1BB). DR-18 can be in the form of a synthetic cytokine that is resistant to the IL-18 binding protein, which usually counteracts the proinflammatory effects of native IL-18 in vivo. In solid tumor models, DR-18 promotes IFN-γ-dependent, CD8⁺ T cell-mediated antitumor responses. This agonist was used for this example because IL-18R gene expression and chromatic accessibility was increased in both CD4⁺ and CD8⁺ T_{SCM} cells (FIG. 4A, FIG. 4B, FIG. 4E, and FIG. 4F). Furthermore, IL-18R is known to be expressed on human memory T cells, and expression of IL-18R on CD4⁺ and CD8⁺ T_{SCM} in patients who underwent allogeneic stem cell transplantation has been confirmed (FIG. 19). The CD137 agonist was used for this example because of its anti-myeloma activity in other preclinical models, and CD137 (Tnfrsf9) was broadly expressed across CD8⁺ T cell clusters irrespective of PT-Cy treatment (FIG. 10).

When administered from 3 days after PT-Cy, both agonist immunotherapies promoted anti-myeloma responses, as evidenced by decreased M-bands at 4 and 6 weeks compared with PT-Cy alone (FIG. 6D). Although GVHD clinical scores were minimally elevated in agonist-treated mice, they remained below those of alloBMT recipients without PT-Cy (FIG. 6D), consistent with the absence of substantial GVHD.

The mechanisms of action of DR-18 and anti-CD137 after PT-Cy were next investigated. A significant increase in the concentration of serum IFN-y was observed and, to a lesser extent, TNF in DR-18- but not anti-CD137-treated recipients compared with PT-Cy alone (FIG. 6E). Immune responses were then tracked in individual mice over time using serial BM aspirates. At 4 weeks after alloBMT, CD8⁺ T cells from PT-Cy-treated mice could be grouped into three populations: nonactivated/bystander (DNAM-1⁺TIGIT⁻ and CD39⁻TIM3⁻), activated/effector (DNAM-1⁺TIGIT⁺ and CD39intTIM3⁻), and exhausted (DNAM-1⁻TIGIT⁺ and CD39hiTIM3⁺) cells. DR-18 preferentially expanded the activated/effector T cell subset, whereas anti-CD137 promoted the exhausted phenotype (FIG. 6F), an outcome possibly driven by the stem-like properties of the CD8⁺ T cells expressing IL-18R. Natural killer (NK) cells were also expanded in DR-18-treated mice (FIG. 6G). There was an almost complete elimination of MM cells from the BM of recipients treated with DR-18 or anti-CD137 by 6 weeks after alloBMT (FIG. 6H). At this 6-week time point, an expansion of CD8⁺ and CD4⁺ T cells was also observed in anti-CD137- treated mice in BM but not in blood (FIG. 6I and FIG. 16A). In DR-18-treated mice, there was no change in T cell numbers; however, the number of DNAM-1⁺ and cytolytic NK cells was significantly increased specifically in the marrow but not in the blood (FIG. 6I and FIG. 6J, and FIG. 16A and FIG. 16B).

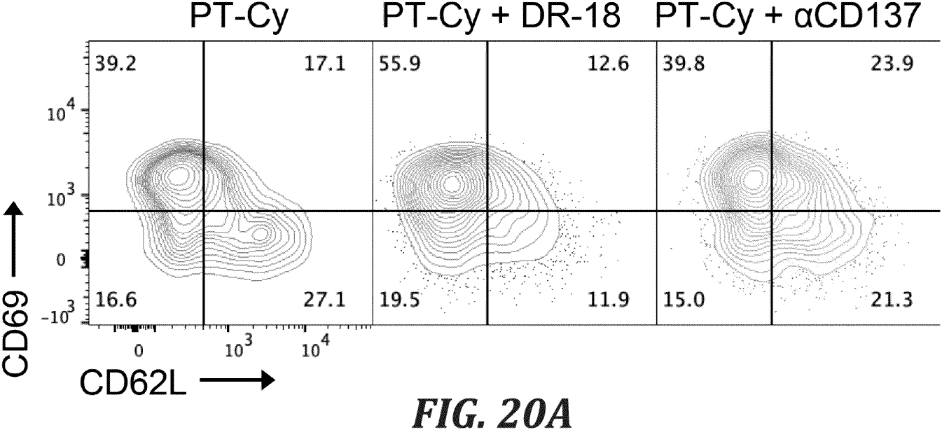

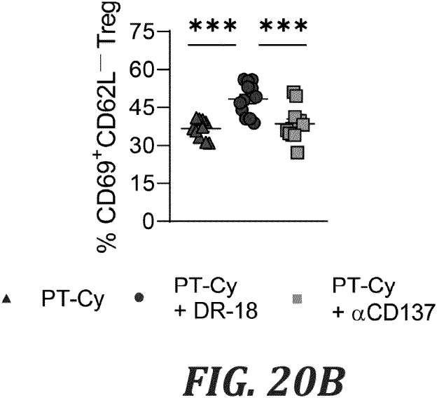

Unbiased clustering of CD4⁺ and CD8⁺ T cell flow cytometry data using FlowSOM revealed differential relative expansion of several immune phenotypes across treatment groups (FIG. 6K and FIG. 6L). Heatmaps depict mean fluorescence intensity (MFI) of each included marker across 12 populations within CD4⁺ T cells (FIG. 17A, FIG. 20A, FIG. 20B). In αCD137-treated mice, regulatory T (Treg) frequency was reduced, whereas exhausted and effector CD4⁺ T cell subsets were increased compared with DR-18-treated and PT-Cy- only recipients (FIG. 17B). DR-18 treatment specifically expanded a CD62L⁻ Treg population (FIG. 17C), suggested to be less suppressive and highly activated, without expanding the overall frequency of Tregs compared with PT-Cy-only recipients. In the CD8⁺ T cell compartment, DR-18 promoted CD8⁺ T cell activation with a relative enrichment in non-exhausted effector populations (FIG. 6L and FIG. 17C) and increased frequency of DNAM- 1+TIGIT⁺ and CD39intTIM3⁻ subsets (FIG. 6M and FIG. 6N) compared with PT-Cy alone recipients. At this time point, CD8⁺ T cells from αCD137-treated recipients were largely terminally exhausted (FIG. 6L, FIG. 6M, FIG. 6N), although the total number of cytotoxic granzyme B⁺ or perforin⁺ CD8⁺ T cells was increased (FIG. 6O and FIG. 6P). In the blood, DR-18 reduced the total number of CD4⁺ and CD8⁺ T cells; however, treatment increased the frequency of T_{EM} in both compartments (FIG. 16A, FIG. 16C, and FIG. 16D). Treatment with αCD137 increased expression of granzyme B, PD-1, CD39, and TOX on CD8⁺ T cells in the blood but not to the same extent as observed in the BM (FIG. 16E). Therefore, agonist immunotherapy promoted immune cell activation, largely in the BM TME, and generated potent myeloma immunity after PT-Cy. DR-18 treatment generated a less terminally exhausted phenotype compared with anti-CD137, potentially due to the expression of IL-18R on stem-like CD8⁺ and CD4⁺ T cells after PT-Cy.

Decoy-resistant IL-18 promotes potent GVL effects

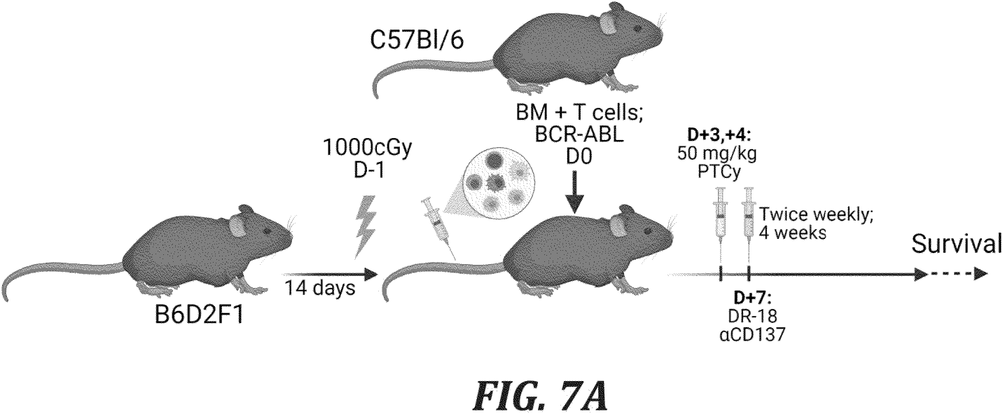

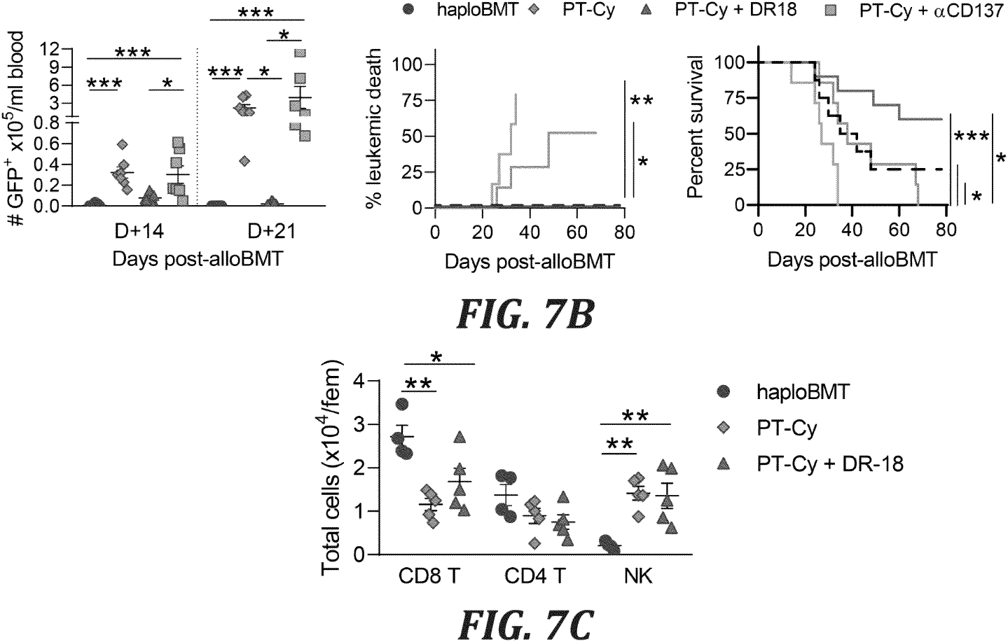

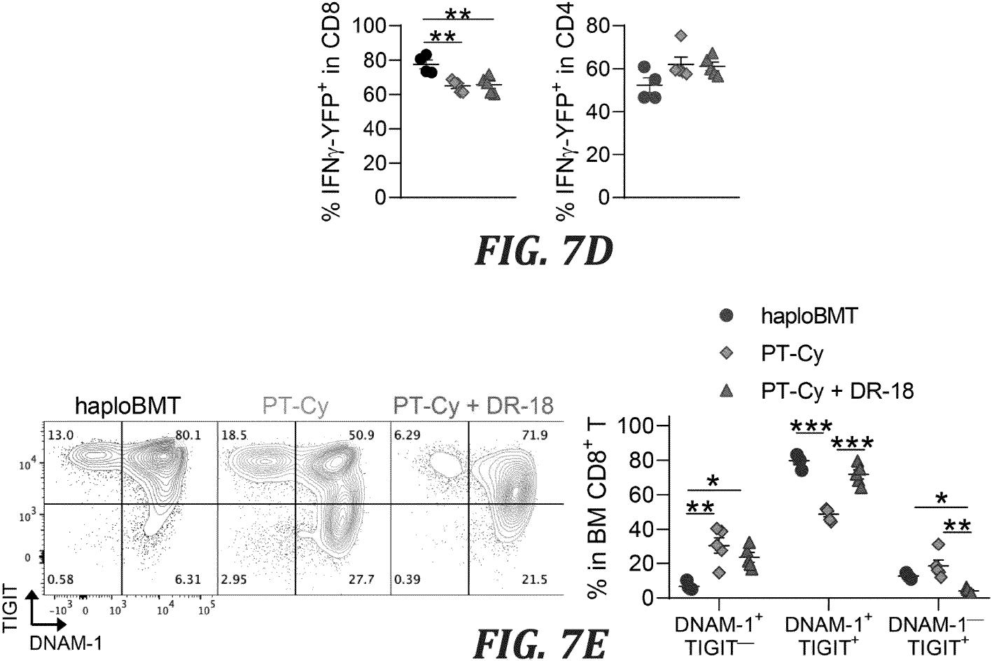

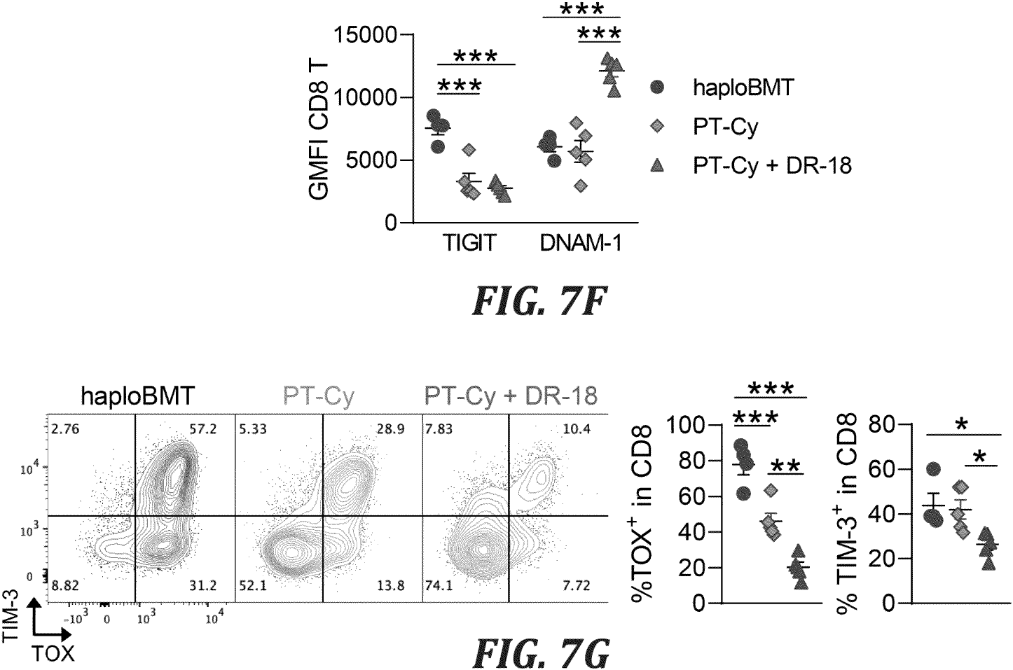

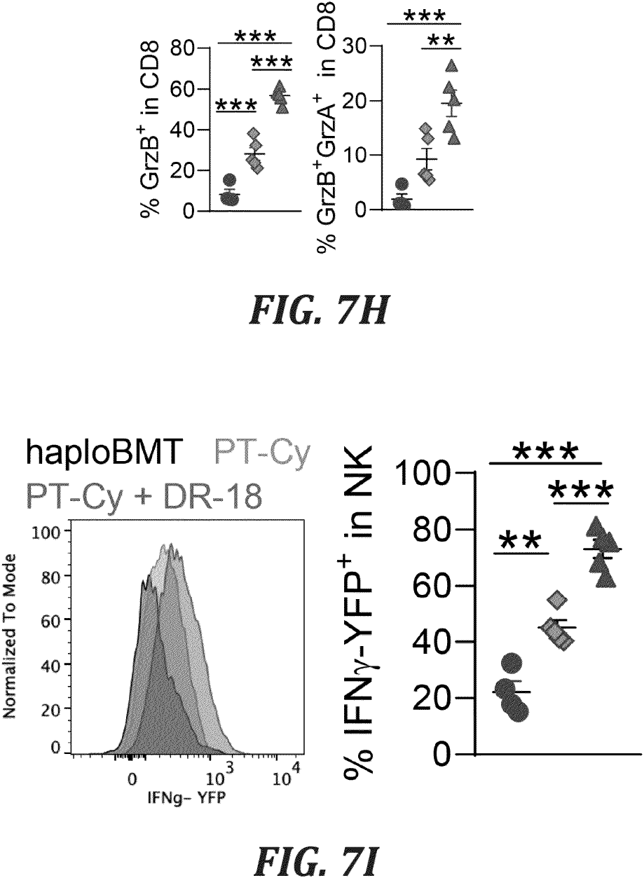

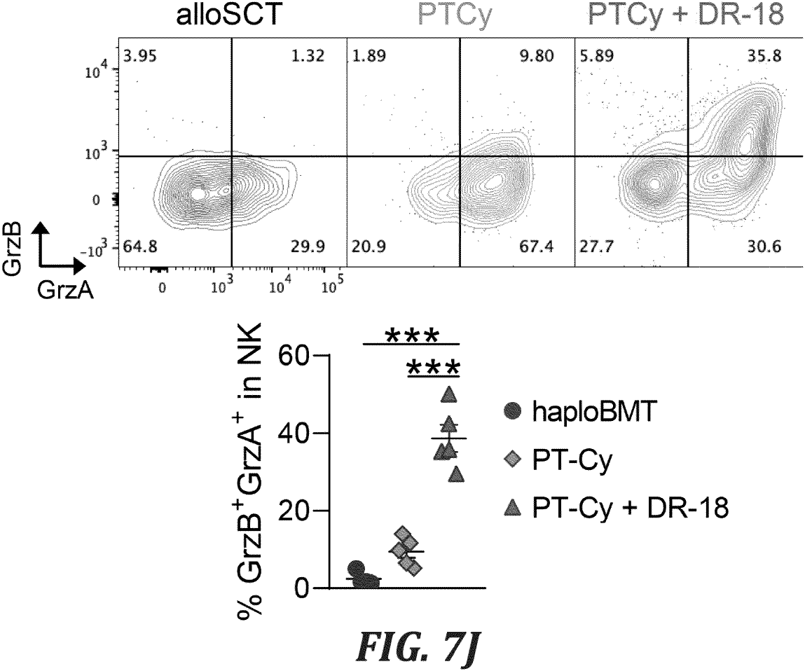

It was next sought to explore the combination of agonist immunotherapy with PT-Cy in a model of haploidentical transplantation (haploBMT), a setting where PT-Cy is a clinical standard of care. In this model, there is a major genetic mismatch between the recipient and the donor whereby lethal GVHD occurs in the absence of any immunosuppressive interventions. Here, a BCR-ABLNUP98- HOXA9 leukemia was used, which is GVL-sensitive (FIG. 7A). In this model, untreated haploBMT recipients developed lethal GVHD, and although PT-Cy reduced the incidence of lethal GVHD, treatment increased relapse-related mortality such that there was no difference in overall survival (FIG. 7B). DR-18 administration after PT-Cy significantly improved GVL responses and overall survival, whereas CD137 agonism had no antitumor efficacy in this model (FIG. 7B).

To explore the mechanisms of DR-18-driven GVL in a haploBMT setting, donor cells from a triple reporter mouse (FoxP3-RFP × IL-10-GFP × IFN-γ-YFP) were used to measure in vivo cytokine production without restimulation. Phenotyping was performed in mice with low leukemia burden to minimize the effect of tumor cells on T cell number in the BM. PT-Cy-treated recipients had reduced CD8⁺ T cell numbers in the BM but increased NK cells compared with untreated haploBMT recipients (FIG. 7C). The frequency of IFN-γ⁺ CD8⁺ T cells was minimally decreased after PTCy, whereas CD4⁺ IFN-γ production was unaffected (FIG. 7D). DR- 18 did not alter IFN-γ production from T cells after PT-Cy (FIG. 7D) but did increase DNAM-1 expression (FIG. 7E and FIG. 7F) and reduced TOX and TIM-3 expression (FIG. 7G) on CD8⁺ T cells. CD8⁺ T cells from DR-18-treated recipients also had significantly increased granzyme B (GrzB) and granzyme A (GrzA) secretion compared with both untreated haploBMT recipients and PT-Cy alone (FIG. 7H). DR-18 also increased the frequency of IFN-γ-producing and GrzA⁺GrzB⁺ NK cells compared with both PT-Cy alone and untreated haploBMT recipients (FIG. 7I and FIG. 7J). Together, these data highlight the ability of DR-18 to drive potent GVL effects by reducing CD8⁺ T cell exhaustion and expanding cytotoxic NK cells after haploBMT with PT-Cy.

### Discussion

Allogeneic BMT is the only curative treatment for many hematological malignancies; however, some malignancies, particularly myeloma, are inherently resistant to GVT effects. Here, murine models of alloBMT that recapitulate these clinical observations were developed to uncover the immunological mechanisms therein. High expression of inhibitory receptors after alloBMT in the absence of tumor antigen, together with the observed lack of GVM but exacerbated GVHD after ICI, suggests that alloantigen primarily drives T cell exhaustion after alloBMT in myeloma. Up-regulation of TIGIT and PD-1 on CD8⁺ T cells after alloBMT presumably enhanced myeloma-mediated suppression of activated alloreactive T cells in a tumor antigen-independent manner, because both PDL1 and CD155 were highly expressed on VK12653 but were largely absent on MLL-AF9-driven AML. The reduction in IFN-y production in CD8⁺ T cells from mice with relapsed AML compared with AML-free mice suggests that these T cells were at a more terminal stage of dysfunction that was driven by tumor antigen. Nonetheless, a study has shown that TIGIT inhibition did not enhance GVL in a preclinical AML model, although anti-PD-1 did provide some antitumor activity. Vk*MYC myeloma expresses clinically relevant inhibitory ligands: both CD155 and PD-L1 have been observed on malignant plasma cells in patients with myeloma. Furthermore, these ligands are expressed on AML cells in some patients and may also contribute to GVT resistance and/or immune escape across several hematological malignancies.

The interaction of inhibitory ligands with their coupled receptors on T cells may inhibit T cell cytolytic activity, reduce effector cytokine production, limit proliferation, and result in T cell apoptosis. Inhibitory receptors are also highly expressed on human CD8⁺ T cells in patients receiving either matched or haploidentical donor grafts. Patients with relapsed disease after matched alloBMT had increased expression of inhibitory receptors on BM CD8⁺ T cells compared with those achieving a complete response, whereas there was no effect of tumor relapse on T cell exhaustion in haploidentical alloBMT recipients. Without wishing to be bound by any particular theory, these clinical observations may corroborate the hypothesis that alloantigen is a key driver of T cell exhaustion after alloBMT. These effects can make subtle changes in T cell exhaustion at relapse difficult to ascertain, and examination of this can be carried out by longitudinal analysis of CD8⁺ T cell subsets by single-cell approaches in large prospective efforts of PT-Cy versus standard immunosuppression in patients who relapse versus those who do not. As a result, subversion of alloreactive T cells by inhibitory ligand expression may be operative in hematological malignancies.

Alloantigen increased the expression not only of inhibitory receptors by donor T cells but also of exhaustion-associated gene signatures, chromatin accessibility within exhaustion-associated genes, and exhaustion-associated motifs. PT-Cy reduced these exhaustion signatures in both CD8⁺ and CD4⁺ T cells and instead enriched for stem-like memory phenotypes and Tcf7-driven motifs. Without wishing to be bound by any particular theory, it is proposed that establishment of a myeloma-driven exhaustion phenotype at relapse enabled agonistic immunotherapy interventions capable of enhancing myeloma-specific immunity without driving the lethal GVHD (as seen after ICI in the absence of PT-Cy).

The inability of ICI to drive myeloma immunity after PT-Cy may be due to cytoreduction of T cells and/or the absence of inhibitory receptor expression on T_{SCM} cells that are specifically enriched after PT-Cy. T_{SCM} cells have been described in the peripheral blood of patients after PT-Cy, and the data demonstrate that T_{SCM} reside in the BM and have high expression of the IL-18R. Furthermore, human T_{SCM} express the IL-18R after alloBMT. DR-18 administration after PT-Cy may act directly on these T_{SCM} cells to promote myeloma immunity and IFN-γ production. IFN-γ secretion by donor CD8⁺ T cells is inversely correlated with their ability to cause GVHD, explaining the absence of lethal GVHD after DR-18. The lack of ICI efficacy and the potent antitumor effects of DR-18 in at least some therapeutic contexts reflects the need for agonists that act as accelerators to drive T cell activation after PT-Cy, which can be contrasted with strategies that block immunological brakes, checkpoints, in cells that are putatively not highly activated at the time of immunotherapy.

The mechanisms of action of PT-Cy have been explored in other studies using very high donor T cell doses and prior PT-Cy treatments that had variable but often lower cyclophosphamide doses compared to those used in this example. These studies demonstrate donor T cell depletion with relative sparing of regulatory T cells. Whether alloreactive T cells are differentially depleted by PT-Cy is less clear, with disparate results depending on the T cell dose and alloreactive T cell clone being tracked. Early and profound depletion of all donor T cells, including alloreactive clones, by PT-Cy has been noted. Likewise, the effect of PT-Cy on GVL, if any, is not clear from prior clinical efforts where patients were transplanted with heterogeneous malignancies and levels of measurable residual disease that limit the power to discriminate effects. The number of T cells to mediate an effective GVL response is substantially lower than that to mediate lethal GVHD, and so, T cell depletion in vivo by PT-Cy does not necessarily mitigate an effective GVL, although it is likely quantitatively modified.

Without wishing to be bound by any particular theory, a recent example provides clinical support for the hypothesis that PT-Cy reduces alloantigen T cell exhaustion and instead facilitates tumor-driven T cell exhaustion. In this example, the authors observed a broad reduction in T cell exhaustion signatures by gene set enrichment analysis in patients treated with PT-Cy, which was then increased in patients who went on to relapse. In the example of this disclosure, it is demonstrated that T cell exhaustion only occurred in the presence of high myeloma burden in the BM after PTCy, despite the fact that alloantigen persists at this site indefinitely through residual recipient stromal cells. Without wishing to be bound by any particular theory, these data suggest that tumor antigen, rather than alloantigen, drives T cell exhaustion after PT-Cy.

Utilization of donor NK cells to enhance GVL is being increasingly studied, particularly in the context of haploidentical transplantation where missing MHC class I and NK-sensitive AML represent favorable immunological contexts to exploit this effect. This is particularly relevant for DR-18 because this cytokine had a stimulatory effect on NK cells, in addition to the effects on CD8⁺ T cells in both the transplantation models and solid tumor systems. Without wishing to be bound by any particular theory, this can be a complementary mechanism of antitumor activity, and it may be likely that the combination of both T and NK cell-mediated GVL is operative. AML is particularly sensitive to NK cell-mediated killing, and this may underlie the lack of efficacy of CD137 agonism in leukemia, because αCD137 did not elicit the same NK cell expansion and activation as was seen with DR-18.

In this example, a novel mechanism of ineffective GVT after alloBMT is identified whereby T cell exhaustion is driven primarily by alloantigen and exacerbation of GVHD does not confer enhanced GVM. Rather, the use of PT-Cy eliminated donor T cell exhaustion signatures and enriched for stem cell memory gene activity early post-alloBMT. This immunophenotype can be targeted with agonistic immunotherapy approaches to enhance GVM and GVL without exacerbating GVHD in both MHC-matched and haploidentical transplantation models. These data provide support for the use of PT-Cy-based immunosuppression as a platform for subsequent agonist immunotherapies after allogenic stem cell transplantation. More broadly, the data demonstrate that stem-like memory T cells are more responsive to agonist immunotherapies than ICI and can be targeted by DR-18 to promote antitumor effects without driving terminal T cell exhaustion.

### Materials and Methods

Example design: This example was designed to interrogate mechanisms behind ineffective GVT responses after allogeneic stem cell transplantation. Murine models that were sensitive or resistant to GVT effects were developed and flow cytometry was used alongside multiomic single-cell RNA sequencing approaches to interrogate CD4 and CD8 T cell phenotypes in the BM. PT-Cy and agonist immunotherapies were then used to overcome GVT resistance and drive potent antitumor responses. Mice were randomly assigned to groups in all experiments without investigator blinding. All n values reflect biological replicates, and numbers of mice per group are included, with the statistical test performed, in the caption for each figure.

Mice: Female C57B⅙ mice were purchased from the Animal Resources Centre (Perth, Western Australia, AUS) or the Jackson Laboratory (Bar Harbor, ME, USA). C3H.SW mice were purchased from the Jackson Laboratory and subsequently bred in house (QIMR Berghofer Medical Research Institute, Brisbane, QLD, Australia; Fred Hutchinson Cancer Center, Seattle, WA, USA). Female B6D2F1 mice were purchased from Charles River and subsequently bred in house (Fred Hutchinson Cancer Center). FoxP3-RFP × IL-10-GFP × IFN-γ-YFP mice were bred in house (Fred Hutchinson Cancer Center). Mice were housed in sterile microisolator cages and received acidified (pH 2.5), autoclaved water and normal chow. Mice were 8 to 12 weeks of age when used in experiments. All animal procedures were performed in accordance with protocols approved by the institutional animal ethics committee.

Stem cell transplantation: Recipient mice were intravenously injected with Vk12653, which originated from Vk*MYC transgenic mice, 2 weeks before BMT (1 × 10⁶ CD138⁺CD19neg cells; MM-bearing mice) or with an MLL-AF9-driven AML (MLL-AF9; 0.1 × 10⁶ GFP⁺) or BCR-ABL-NUP98-HOXA9 (1.0 × 10⁶ GFP⁺) on D0 (AML-bearing mice). Recipients were transplanted with BM and T cell grafts (doses detailed in the figure legends) administered via tail vein injection the day after lethal irradiation (1000 cGy for C57B⅙ and 1100 cGy for B62DF1, 137Cs source). Every 2 weeks, serum samples were collected from MMbearing recipients, and M-band was quantified using a Sebia Hydrasys serum protein electrophoresis system (HYDRASYS 2 Scan). Leukemia cell number in blood was calculated weekly using flow cytometry to quantify GFP⁺ cells in blood. Recipients were monitored daily, up to 120 days after BMT, and euthanized when hindlimb paralysis occurred or clinical scores reached ≥6. In competing risk analyses, deaths were attributed to myeloma if the M-band was above 0.28, a defined relapse threshold. In the leukemia models, leukemic death was defined by a white blood cell count above 50 × 10⁶/ml or a GFP⁺ leukemia frequency above 50% in blood or BM. For some experiments, mice were treated with 100 µg of anti-TIGIT monoclonal antibody (mAb; 4B1, Bristol Myers Squibb) or mouse IgG2a (anti-KLH) twice a week for 4 weeks from D+14 postalloBMT. For other experiments, mice were treated with 100 µg of Fc-dead anti-TIGIT mAb (D265A, Bristol Myers Squibb) or mouse IgG1 (anti-KLH.1) twice a week for 6 weeks from D+21 postalloBMT. Cyclophosphamide (Fisher Scientific; 99.5%, MP Biomedicals) was intraperitoneally administered at 50 mg/kg on D+3 and D+4 after alloBMT (PT-Cy). After PT-Cy, 100 µg of anti-TIM- 3 mAb (RMT3-23, Bio X Cell), anti-PD-1 mAb (RMP1-14, Bio X Cell), or anti-CD137 (4-1BB, 3H3, Bio X Cell) and related isotypes were intraperitoneally administered twice a week, whereas 8 µg of DR-18 was subcutaneously administered twice a week from D+7 for 4 weeks. DR-18 was supplied by Simcha Therapeutics (New Haven, CT).

Cell preparation for flow cytometry: Recipient mice were euthanized 2 to 8 weeks after transplant, and cells from BM or blood were harvested. For BM aspirates, mice were anesthetized and treated with a local analgesic (0.5% lidocaine) followed by injection of 30 µl of phosphate-buffered saline (PBS) into the femur to allow up to 10 µl of marrow to be aspirated for fluorescence- activated cell sorting (FACS) analysis. For surface marker phenotyping, isolated cells were incubated with Fc-block before staining with fluorescently tagged antibodies (listed in Table 1), on ice for 30 min. For intracellular staining, cells were surface labeled, fixed, and permeabilized (eBioscience, Foxp3 Transcription Factor Staining Buffer Kit) before intracellular staining at room temperature for 60 min. To measure cytokine production, cells were stimulated for 4 hours at 37° C. with PMA (500 ng/ml) and ionomycin (50 ng/ml; Sigma-Aldrich) with brefeldin A (BioLegend). All samples were acquired on a BD LSR Fortessa (BD Biosciences) or BD FACSymphony A3 (BD Biosciences) and analyzed using FlowJo software (v10). t-SNE analysis was performed using the FlowJo plugin with default settings on a concatenated sample with 3000 CD8 T cells per mouse. FlowSOM analysis was performed with 3000 to 4000 CD8 or CD4 T cells per mouse concatenated after downsampling.

TABLE 1

Flow cytometry antibodies

| | | | |
|---|---|---|---|
| Marker | Clone | Fluorochrome | Company |
| CD155 | 4.24.1 | PE | Biolegend |
| CD138 | 281-2 | PE, BV421 | Biolegend |
| CD226 | TX42.1 | AF647, BV650 | Biolegend |
| CD101 | RM101 Moushi101 | AF647 AF700 | Biolegend eBioscience |
| CD69 | H1.2F3 | FITC, BV786 | Biolegend |
| CD62L | MEL-14 | AF700 BV480 | Biolegend BD Bioscience |
| CD4 | GK1.5 | AF700 BUV496 | Biolegend BD Bioscience |
| CD3 | 145-2C11 | BV711 | Biolegend |
| CD274 | 10F.9G2 | APC | Biolegend |
| CD19 | 6D5 | APC-Cy7 | Biolegend |
| CD38 | 90 T10 | APC-Cy7 PE-Cy7 | Biolegend |
| CD8 | 53-6.7 | APC-Cy7 BUV805 | Biolegend BD Bioscience |
| PD-1 | 29F.1A12 RMP1-30 J43 | BV421 PE-Cy7 BUV737 | Biolegend Biolegend BD Bioscience |
| CD44 | IM7 | BV421, APC-Cy7 | Biolegend |
| CD90.2 | 53-2.1 | BV605 | Biolegend |
| TIM-3 | RMT3-23 | PE, FITC BV605 | EBioscience Biolegend |
| TIGIT | GIGD7 1G9 | PerCp-eFluor710 BV421 | eBioscience BD Bioscience |
| NKp46 | 29A1.4 | PE | Biolegend |
| Ly108 | 13G3 | BUV661 | BD Bioscience |
| TOX | TXRX10 | eFluor660 | eBioscience |
| CD39 | Duha59 | PE-Cy7 | Biolegend |
| FoxP3 | FJK-16s | PE-Cy5 | eBioscience |
| NRP-1 | 3E12 | PerCp/Cy5.5 | Biolegend |
| CD49b | HMα2 | BUV563 | BD Bioscience |
| CXCR3 | CXCR3-173 | BV605 | Biolegend |
| TCF1 | S33-966 | PE | BD Bioscience |
| Granzyme B | QA16A02 | PE-Dazzle594 | Biolegend |
| Perforin | S16009A | PE | Biolegend |
| CD122 | TM-β1 | BB700 | BD Bioscience |
| CD45 | 30-F11 | Buv395 | BD Bioscience |

Single-cell RNA/ATAC sequencing: Naive C57B⅙ mice were transplanted with C3H.SW grafts and were untreated (alloBMT) or treated with PT-Cy (50 mg/kg) on D+3 and D+4 (PT-Cy). BM was harvested from femurs (four mice pooled per group) at D+14 after alloBMT, and T cells were sort-purified (CD90.2⁺CD4⁺ and CD90.2⁺CD8⁺) before nuclei preparation according to the 10x Genomics Multiome protocol. Nuclei (also referred to as cells herein) were captured and libraries were generated according to the manufacturer’s specifications. Libraries were sequenced using Illumina NovaSeq 6000 targeting a depth of 25,000 reads per cell per library.

Single-cell RNA/ATAC analysis: Reads were demultiplexed and processed using cellranger-arc v1.0.1 aligning reads to GENCODE vM23/Ensembl98. Peaks were called from each sample’s fragment file (cellranger output) using MACS2 using the parameters “--nomodel --extsize 200 --shift -100.” Quantification of reads in MACS2 peaks were calculated and integrated with cellranger RNA output using Signac. Cells meeting the following criteria (calculated using Signac) were retained for downstream analysis: percent mitochondrial RNA reads <10%; 3 > log10(ATAC counts) < 4.5; 3 > log10(RNA/UMI counts) < 4.5; fraction of reads in peaks >40%; transcription start site (TSS) percentile >75%. RNA/unique molecular identifier (UMI) counts were subject to a variance-stabilized normalization procedure using Seurat’s “glmGamPoi” function before dimensionality reduction using principal components analysis (PCA). ATAC data after TFIDF/SVD dimensionality reduction were integrated with reduced-dimensionality RNA data (PCA matrix) using the WNN function with default parameters. CD4 and CD8 cells were defined using absolute RNA/UMI counts greater than 0. Cells with counts for both CD4 and CD8 were further excluded. Clusters were identified using the standard Seurat workflow. Gene activity scores and Motif scores were calculated using Signac and chromVAR.

External data: To identify genes specific for T_{SCM} cells, publicly available single-cell RNA sequencing data were obtained from Gene Expression Omnibus (GSE152379) and processed using Seurat. To generate a high-confidence list of T_{SCM}-specific genes, those genes specific to BACH2-overexpressing cells were identified using a strict filter of a q value of <0.001 and log2 fold change of >1, removing ribosomal protein genes (Rpl* and Rps*), and included critical T_{SCM} genes, Bach2, Bcl2, Eomes, Myb, and Tnfsf8. Gene set for exhaustion (T_{EX}) was generated taking data from published bulk expression profiles and filtered using the same cutoffs. Gene set scores were calculated using the AddModuleScore function in Seurat. Statistical test for gene set scores was calculated using Wilcoxon rank sum in R.

Human samples: Peripheral blood mononuclear cells from an Institutional Review Board-approved example of immune reconstitution in patients receiving allogeneic stem cell transplantation at the Fred Hutchinson Cancer Center were thawed and resuspended in prewarmed culture medium containing deoxyribonuclease (DNase) 1. Cells were washed twice with PBS before incubating with FVS440UV (BD Biosciences) and Fc Block (Human TruStain, BioLegend) for 15 min at room temperature. Cells were washed and then stained with surface flow cytometry antibodies for 30 min on ice. Cells were washed and fixed with eBioscience FoxP3 staining kit according to the manufacturer’s protocol before intracellular staining at room temperature for 1 hour.

Statistical analysis: Data are presented as means ± SEM, and P < 0.05 was considered significant. Survival curves were plotted using Kaplan-Meier estimates and compared by log-rank (Mantel-Cox) test. M-bands were modeled, and the M-band relapse threshold (G/A above 0.282) utilized. Competing risk analysis was performed using the cmprsk R package. Comparisons between two groups were performed with t test or Mann-Whitney U test, and comparisons between three or more groups were performed with one-way analysis of variance (ANOVA) and Tukey’s multiple comparisons test for normally distributed data or with Kruskal-Wallis and Dunn’s multiple comparisons test for nonparametric data.

### Specific Embodiments

Embodiment 1. A method for treating a hematological malignancy in a subject, the method comprising: transplanting a tissue that comprises a plurality of T cells to the subject; and stimulating the plurality of T cells to enhance a graft-versus-tumor (GVT) response of the plurality of T cells to treat the hematological malignancy.

Embodiment 2. The method of Embodiment 1, wherein the subject has a probability of graft-versus-host disease (GVHD) and/or relapse of the hematological malignancy and the method reduces the probability of GVHD and/or relapse of the hematological malignancy.

Embodiment 3. The method of any of Embodiments 1-2, wherein the tissue includes bone marrow or blood stem cells and is allogeneic to the subject.

Embodiment 4. The method of any of Embodiments 1-3, further comprising enriching for a stem-like memory T cell phenotype in the plurality of T cells.

Embodiment 5. The method of any of Embodiments 1-4, wherein the enriching comprises depleting at least a portion of the plurality of T cells.

Embodiment 6. The method of any of Embodiments 1-5, wherein the enriching comprises administering a post-transplant cyclophosphamide (PT-Cy) treatment to the subject.

Embodiment 7. The method of any of Embodiments 1-6, wherein the administering depletes at least a portion of an alloreactive portion of the plurality of T cells to enrich for the stem-like memory T cell phenotype in the plurality of T cells.

Embodiment 8. The method of any of Embodiments 1-7, wherein the stem-like memory T cell phenotype comprises an increased chromatin accessibility in a cytokine signaling gene and/or an increased expression of interleukin-18 receptor (IL-18R), Transcription Factor 7 (TCF7), Transcription Factor 7 Like 2 (TCFL2), Krüppel-like transcription factor 2 (KLF2), Krüppel-like transcription factor 4 (KLF4), and/or Krüppel-like transcription factor 5 (KLF5) by at least a portion of the plurality of T cells.

Embodiment 9. The method of any of Embodiments 1-8, wherein the stimulating comprises administering an agonist immunotherapy to the subject for expansion of CD8 T cells, expansion of natural killer (NK) cells, or both.

Embodiment 10. The method of any of Embodiments 1-9, wherein the stimulating comprises administering an anti-CD137 antibody treatment to the subject.

Embodiment 11. The method of any of Embodiments 1-10, wherein the stimulating comprises administering a decoy-resistant IL-18 (DR-18) treatment to the subject.

Embodiment 12. The method of any of Embodiments 1-11, further comprising administering a donor lymphocyte infusion (DLI) to the subject.

Embodiment 13. A method for treating a hematological malignancy in a subject, the method comprising: transplanting a tissue that comprises a plurality of T cells to the subject; administering a post-transplant cyclophosphamide (PT-Cy) treatment to the subject to deplete at least a portion of the plurality of T cells and enrich for a stem-like memory T cell phenotype in the plurality of T cells; and administering an agonist immunotherapy to the subject to enhance a graft-versus-tumor (GVT) response of the plurality of T cells to treat the hematological malignancy.

Embodiment 14. The method of Embodiment 13, wherein the tissue includes bone marrow or blood stem cells.

Embodiment 15. The method of any of Embodiments 13-14, wherein the bone marrow or blood stem cells is allogeneic to the subject.

Embodiment 16. The method of any of Embodiments 13-15, wherein the administering the agonist immunotherapy comprises administering an anti-CD137 antibody treatment to the subject.

Embodiment 17. The method of any of Embodiments 13-16, wherein the administering the agonist immunotherapy comprises administering a decoy-resistant IL-18 (DR-18) treatment to the subject.

Embodiment 18. The method of any of Embodiments 13-17, further comprising administering a donor lymphocyte infusion (DLI) to the subject.

Embodiment 19. A method for enhancing a graft-versus-tumor (GVT) response of a plurality of T cells to treat a hematological malignancy of a subject, the method comprising: administering a decoy-resistant IL-18 (DR-18) treatment to the subject.

Embodiment 20. The method of Embodiment 19, further comprising: administering a treatment to the subject to enrich for a stem-like memory T cell phenotype in the plurality of T cells.

While illustrative embodiments have been illustrated and described, it will be appreciated that various changes can be made therein without departing from the spirit and scope of the disclosure.

The embodiments of the invention in which an exclusive property or privilege is claimed are defined as follows:

1. A method for treating a hematological malignancy in a subject, the method comprising:
transplanting a tissue that comprises a plurality of T cells to the subject; and
stimulating the plurality of T cells to enhance a graft-versus-tumor (GVT) response of the plurality of T cells to treat the hematological malignancy.

2. The method of claim 1, wherein the subject has a probability of graft-versus-host disease (GVHD) and/or relapse of the hematological malignancy and the method reduces the probability of GVHD and/or relapse of the hematological malignancy.

3. The method of claim 1, wherein the tissue includes bone marrow or blood stem cells and is allogeneic to the subject.

4. The method of claim 1, further comprising enriching for a stem-like memory T cell phenotype in the plurality of T cells.

5. The method of claim 4, wherein the enriching comprises depleting at least a portion of the plurality of T cells.

6. The method of claim 4, wherein the enriching comprises administering a post-transplant cyclophosphamide (PT-Cy) treatment to the subject.

7. The method of claim 6, wherein the administering depletes at least a portion of an alloreactive portion of the plurality of T cells to enrich for the stem-like memory T cell phenotype in the plurality of T cells.

8. The method of claim 1, wherein the stem-like memory T cell phenotype comprises an increased chromatin accessibility in a cytokine signaling gene and/or an increased expression of interleukin-18 receptor (IL-18R), Transcription Factor 7 (TCF7), Transcription Factor 7 Like 2 (TCFL2), Krüppel-like transcription factor 2 (KLF2), Krüppel-like transcription factor 4 (KLF4), and/or Krüppel-like transcription factor 5 (KLF5) by at least a portion of the plurality of T cells.

9. The method of claim 1, wherein the stimulating comprises administering an agonist immunotherapy to the subject for expansion of CD8 T cells, expansion of natural killer (NK) cells, or both.

10. The method of claim 1, wherein the stimulating comprises administering an anti-CD137 antibody treatment to the subject.

11. The method of claim 1, wherein the stimulating comprises administering a decoy-resistant IL-18 (DR-18) treatment to the subject.

12. The method of claim 1, further comprising administering a donor lymphocyte infusion (DLI) to the subject.

13. A method for treating a hematological malignancy in a subject, the method comprising:
transplanting a tissue that comprises a plurality of T cells to the subject;
administering a post-transplant cyclophosphamide (PT-Cy) treatment to the subject to deplete at least a portion of the plurality of T cells and enrich for a stem-like memory T cell phenotype in the plurality of T cells; and
administering an agonist immunotherapy to the subject to enhance a graft-versus-tumor (GVT) response of the plurality of T cells to treat the hematological malignancy.

14. The method of claim 13, wherein the tissue includes bone marrow or blood stem cells.

15. The method of claim 14, wherein the bone marrow or blood stem cells is allogeneic to the subject.

16. The method of claim 13, wherein the administering the agonist immunotherapy comprises administering an anti-CD137 antibody treatment to the subject.

17. The method of claim 13, wherein the administering the agonist immunotherapy comprises administering a decoy-resistant IL-18 (DR-18) treatment to the subject.

18. The method of claim 13, further comprising administering a donor lymphocyte infusion (DLI) to the subject.

19. A method for enhancing a graft-versus-tumor (GVT) response of a plurality of T cells to treat a hematological malignancy of a subject, the method comprising:
administering a decoy-resistant IL-18 (DR-18) treatment to the subject.

20. The method of claim 19, further comprising:
administering a treatment to the subject to enrich for a stem-like memory T cell phenotype in the plurality of T cells.
